(19) United States
(12) Patent Application Publication
(10) Pub. No.: US 2016/0274108 A1
(43) Pub. Date: Sep. 22, 2016

(54) DIAGNOSTIC METHODS FOR PAR4 ANTAGONIST THERAPY

(71) Applicants: BRISTOL-MYERS SQUIBB COMPANY, Princeton, NJ (US); CAMBRIDGE ENTERPRISE LIMITED, Cambridge (GB)

(72) Inventors: Jing YANG, Princeton, NJ (US); Willem H. OUWEHAND, Cambridge (GB)

(21) Appl. No.: 15/031,941

(22) PCT Filed: Oct. 24, 2014
(86) PCT No.: PCT/US14/62251

§ 371 Date: Apr. 25, 2016

Related U.S. Application Data
(60) Provisional application No. 61/895,968, filed on Oct. 25, 2013.

(51) Int. Cl. G01N 33/566; A61K 38/03

(52) U.S. Cl. CPC G01N 33/566; A61K 38/03; G01N 2500/10; G01N 2333/705

(57) ABSTRACT

The disclosure provides methods for identifying a PAR4 agonist responder or a PAR4 antagonist responder comprising contacting a PAR4 agonist with platelets in a sample obtained from a subject in need thereof. The methods also include administering a PAR4 antagonist to the subject identified as a PAR4 agonist responder or a PAR4 antagonist responder. The invention also includes methods of treating the subjects and a pharmaceutical kit comprising a PAR4 agonist.

### BACKGROUND OF THE DISCLOSURE

1. Field of the Disclosure

The present disclosure provides methods for identifying a subject who is suitable for PAR4 antagonist therapy. The present disclosure also provides methods for adjusting doses of a PAR4 antagonist for treating a disease or condition associated with thromboembolism.

2. Background Art

Thromboembolic diseases remain the leading cause of death in developed countries despite the availability of anticoagulants such as warfarin (COUMADIN®), heparin, low molecular weight heparins (LMWH), synthetic pentasaccharides, and antiplatelet agents such as aspirin and clopidogrel (PLAVIX®).

Current anti-platelet therapies have limitations including increased risk of bleeding as well as partial efficacy (relative cardiovascular risk reduction in the 20% to 30% range). Thus, discovering and developing safe and efficacious oral or parenteral antithrombotics for the prevention and treatment of a wide range of thromboembolic disorders remains an important goal.

A dual anti-platelet treatment with clopidogrel and aspirin has been established as the standard of care for medical and interventional management of acute coronary syndromes. Randomized controlled trials show that the standard of care treatment is variable both between patients and across multiple measurements within a patient, with some patients showing no or minimal platelet response to the clopidogrel administration.

Indeed, several studies have confirmed the long-held notion that the response of platelets to agonists is highly variable within the general population, while the level of responsiveness is remarkably consistent over time for an individual. See Panzer, S., et al., Ann Hematol. 85: 121-125 (2006). Another study has found that certain healthy individuals consistently demonstrate platelet hyperreactivity. See Yee, D., et al., Blood, 106: 2723-2729 (2005). These results imply high variability in platelet response to antiplatelet drugs among the patients. Therefore, to improve patients' responsiveness to antithrombotics, there is a need to identify the patients who would respond well to a particular antithrombotics.

### BRIEF SUMMARY OF THE DISCLOSURE

The present invention provides a method for identifying a PAR4 agonist responder comprising contacting a PAR4 agonist with platelets in a sample obtained from a subject in need thereof, wherein the PAR4 agonist activates the platelets and comprises an amino acid sequence of Formula I:

Ala-Xₐₐ₁-Pro-Gly-Xₐₐ₂-Leu-Val (Formula I)
wherein,
the amino terminus of the peptide is not fused to an amino acid;
Xₐₐ₁ is selected from Tyr or Phe(4-F);
Xₐₐ₂ is selected from Trp(5-OH), (D,L)-Trp(5-Br), D-Trp, Bzt, Tpi, His, Tza, 3-Thi, 3-Fur, His(Bzl), Phe, Tyr, Phe(penta-F), 2-Pya, 3-Pya, 4-Pya, Dpa, 3-Pya(4-Tolyl), Bip(2-Methyl), 1-Naphthyl-Ala, 2-Naphthyl-Ala, Tyr(Bzl), or Styryl-Ala; and
(−) is a peptide bond; and
the peptide has a PAR4 agonist activity.

The invention also provides a method for identifying a PAR4 antagonist therapy responder comprising contacting a PAR4 agonist with platelets in a sample obtained from a subject in need of PAR4 antagonist therapy, wherein the PAR4 agonist activates the platelets and comprises an amino acid sequence of Formula I:

Ala-Xₐₐ₁-Pro-Gly-Xₐₐ₂-Leu-Val (Formula I)
wherein,
the amino terminus of the peptide is not fused to an amino acid;
Xₐₐ₁ is selected from Tyr or Phe(4-F);
Xₐₐ₂ is selected from Trp(5-OH), (D,L)-Trp(5-Br), D-Trp, Bzt, Tpi, His, Tza, 3-Thi, 3-Fur, His(Bzl), Phe, Tyr, Phe(penta-F), 2-Pya, 3-Pya, 4-Pya, Dpa, 3-Pya(4-Tolyl), Bip(2-Methyl), 1-Naphthyl-Ala, 2-Naphthyl-Ala, Tyr(Bzl), or Styryl-Ala; and
(−) is a peptide bond; and
the peptide has a PAR4 agonist activity, and
wherein platelet activation indicates that the subject is a PAR4 antagonist therapy responder.

In one embodiment, the invention provides a method for evaluating platelet activation by a PAR4 agonist in a subject in need thereof comprising (i) contacting a PAR4 agonist with platelets in a sample obtained from the subject, wherein the PAR4 agonist activates the platelets and comprises an amino acid sequence of Formula I:

Ala-Xₐₐ₁-Pro-Gly-Xₐₐ₂-Leu-Val (Formula I)
wherein,
the amino terminus of the peptide is not fused to an amino acid;
Xₐₐ₁ is selected from Tyr or Phe(4-F);
Xₐₐ₂ is selected from Trp(5-OH), (D,L)-Trp(5-Br), D-Trp, Bzt, Tpi, His, Tza, 3-Thi, 3-Fur, His(Bzl), Phe, Tyr, Phe(penta-F), 2-Pya, 3-Pya, 4-Pya, Dpa, 3-Pya(4-Tolyl), Bip(2-Methyl), 1-Naphthyl-Ala, 2-Naphthyl-Ala, Tyr(Bzl), or Styryl-Ala; and
(−) is a peptide bond; and
the peptide has a PAR4 agonist activity
and (ii) measuring activation of the platelets.

In another embodiment, the invention includes a method for evaluating responsiveness of a subject to PAR4 antagonist therapy comprising (i) contacting a PAR4 agonist with platelets in a sample obtained from the subject in need of PAR4 antagonist therapy, wherein the PAR4 agonist activates the platelets and comprises an amino acid sequence of Formula I:

Ala-Xₐₐ₁-Pro-Gly-Xₐₐ₂-Leu-Val (Formula I)
wherein,
the amino terminus of the peptide is not fused to an amino acid;
Xₐₐ₁ is selected from Tyr and Phe(4-F);
Xₐₐ₂ is selected from Trp(5-OH), (D,L)-Trp(5-Br), D-Trp, Bzt, Tpi, His, Tza, 3-Thi, 3-Fur, His(Bzl), Phe, Tyr, Phe(penta-F), 2-Pya, 3-Pya, 4-Pya, Dpa, 3-Pya(4-Tolyl), Bip(2-Methyl), 1-Naphthyl-Ala, 2-Naphthyl-Ala, Tyr(Bzl), or Styryl-Ala; and
(−) is a peptide bond; and
the peptide has a PAR4 agonist activity, and
(ii) measuring the platelet activation,
wherein platelet activation equal to or above a normal diagnostic score and below a high diagnostic score (i.e., normal responder or high responder to the PAR4 agonist) indicates that the subject is a normal candidate for PAR4 antagonist therapy.

In other embodiments, platelet activation above a high diagnostic score (i.e., high responder to the PAR4 agonist) indicates that the subject is an optimal candidate for PAR4 antagonist therapy. In still other embodiments, platelet activation above no response below a normal diagnostic score (i.e., low responder to the PAR4 agonist) indicates that the subject can be a candidate for PAR4 antagonist therapy. In certain embodiments, no platelet activation indicates that the subject is not a candidate for PAR4 antagonist therapy.

In other embodiments, the invention provides a method for treating, preventing, or ameliorating a disease or condition associated with thromboembolism in a subject in need thereof comprising (i) submitting a platelet sample obtained from the subject for platelet activation testing with a PAR4 agonist, and (ii) administering an effective amount of a PAR4 antagonist to the subject wherein the effective amount is determined based on the result of the platelet activation testing and wherein the PAR4 agonist comprises an amino acid sequence of Formula I:

Ala-Xₐₐ₁-Pro-Gly-Xₐₐ₂-Leu-Val (Formula I)
wherein,
the amino terminus of the peptide is not fused to an amino acid;
Xₐₐ₁ is selected from Tyr and Phe(4-F);
Xₐₐ₂ is selected from Trp(5-OH), (D,L)-Trp(5-Br), D-Trp, Bzt, Tpi, His, Tza, 3-Thi, 3-Fur, His(Bzl), Phe, Tyr, Phe(penta-F), 2-Pya, 3-Pya, 4-Pya, Dpa, 3-Pya(4-Tolyl), Bip(2-Methyl), 1-Naphthyl-Ala, 2-Naphthyl-Ala, Tyr(Bzl), or Styryl-Ala; and
(−) is a peptide bond; and
the peptide has a PAR4 agonist activity.

Some embodiments of the invention include a method for reducing or decreasing platelet aggregation in a subject in need thereof comprising administering an effective amount of a PAR4 antagonist to the subject wherein the effective amount is determined based on the level of platelet activation when the platelets of the subject are activated by a PAR4 agonist, which comprises an amino acid sequence of Formula I:

Ala-Xₐₐ₁-Pro-Gly-Xₐₐ₂-Leu-Val (Formula I)
wherein,
the amino terminus of the peptide is not fused to an amino acid;
Xₐₐ₁ is selected from Tyr and Phe(4-F);
Xₐₐ₂ is selected from Trp(5-OH), (D,L)-Trp(5-Br), D-Trp, Bzt, Tpi, His, Tza, 3-Thi, 3-Fur, His(Bzl), Phe, Tyr, Phe(penta-F), 2-Pya, 3-Pya, 4-Pya, Dpa, 3-Pya(4-Tolyl), Bip(2-Methyl), 1-Naphthyl-Ala, 2-Naphthyl-Ala, Tyr(Bzl), or Styryl-Ala; and
(−) is a peptide bond; and
the peptide has a PAR4 agonist activity.

Other embodiments of the invention provide a method for treating, preventing, or ameliorating a disease or condition associated with thromboembolism in a subject in need thereof comprising administering an effective amount of a PAR4 antagonist to the subject wherein the effective amount is determined based on the level of platelet activation when the platelets of the subject are activated by a PAR4 agonist, which comprises an amino acid sequence of Formula I:

Ala-Xₐₐ₁-Pro-Gly-Xₐₐ₂-Leu-Val (Formula I)
wherein,
the amino terminus of the peptide is not fused to an amino acid;
Xₐₐ₁ is selected from Tyr and Phe(4-F);
Xₐₐ₂ is selected from Trp(5-OH), (D,L)-Trp(5-Br), D-Trp, Bzt, Tpi, His, Tza, 3-Thi, 3-Fur, His(Bzl), Phe, Tyr, Phe(penta-F), 2-Pya, 3-Pya, 4-Pya, Dpa, 3-Pya(4-Tolyl), Bip(2-Methyl), 1-Naphthyl-Ala, 2-Naphthyl-Ala, Tyr(Bzl), or Styryl-Ala; and
(−) is a peptide bond; and
the peptide has a PAR4 agonist activity.

In some embodiments of the invention, the categorization of subjects can include a PAR4 antagonist inhibitor test. In some embodiments, measurement of a subject's response to PAR4 antagonist comprises (i) obtaining a blood sample from a subject that has not been treated with a PAR4 antagonist, (ii) pre-incubating a first fraction of the sample with a PAR4 antagonist while the second fraction is not pre-incubated with the PAR4 antagonist, (iii) treating platelets from the first fraction and the second fraction of the blood sample with a PAR4 agonist in vitro and (iv) measuring platelet activation of both the first and second fractions. The method further comprises (v) determining the percentage inhibition of platelet activation by the PAR4 antagonist by comparing the platelet activation in the first fraction with the platelet activation in the second fraction. In certain embodiments, if the sample obtained after PAR4 antagonist treatment has low reactivity for PAR4 agonist (a high percentage of inhibition of PAR4 agonist by PAR4 antagonist), then the test indicates that the PAR4 antagonist treatment is working well for the subject. In certain embodiments, if the sample obtained after PAR4 antagonist treatment has high reactivity for PAR4 agonist (a low percentage of inhibition of PAR4 agonist by PAR4 antagonist), then the test indicates that the PAR4 antagonist treatment may not be as effective for the subject and a dose adjustment can be required.

One embodiment of the invention provides a method for treating a subject with a PAR4 antagonist, the method comprising: (i) obtaining a blood sample from the subject (ii) contacting a PAR4 agonist with platelets in the sample obtained from the subject, (iii) measuring activation of the platelets, (iv) determining if the subject is a high responder, a normal responder, a low responder or a non-responder to the PAR4 agonist based on the amount of platelet activation observed, and (v) administering a PAR4 antagonist to the subject if the subject is a high responder, a normal responder or a low responder to the PAR4 agonist.

Another embodiment of the invention provides a method for treating a subject with a PAR4 antagonist, the method comprising: (i) obtaining a blood sample from the subject (ii) contacting a PAR4 agonist with platelets in the sample obtained from the subject, (iii) measuring activation of the platelets (iv) determining if the subject is a high responder, a normal responder, a low responder or a non-responder to PAR4 agonist based on the amount of platelet activation observed, and (v) providing a report to a healthcare provider recommending that the healthcare provider treat the subject with a PAR4 antagonist if the subject is a high responder, a normal responder or a low responder.

Another embodiment of the invention provides a method for treating a subject with a PAR4 antagonist, the method comprising: (i) obtaining a blood sample from a subject that has not been treated with the PAR4 antagonist, (ii) pre-incubating a first fraction of the sample with a PAR4 antagonist while a second fraction is not pre-incubated with the PAR4 antagonist, (iii) treating platelets from the first fraction and the second fraction of the blood sample with a PAR4 agonist in vitro, (iv) measuring platelet activation of both the first and second fractions, (v) determining the percentage inhibition of platelet activation by the PAR4 antagonist by comparing the platelet activation in the first fraction with the platelet activation in the second fraction, and (vi) treating the subject with a PAR4 antagonist if the second fraction has little to no platelet activation compared to the first fraction.

In certain embodiments, platelet activation is measured by changes in the platelet cytoplasm, by changes of the platelet membrane, by changes in the levels of analytes released by platelets, by the changes in the morphology of the platelet, by the ability of platelets to form thrombi or platelet aggregates in flowing or stirred whole blood, by the ability of platelets to adhere to a static surface which is derivatized with relevant ligands (e.g., von Willebrand Factor, collagen, fibrinogen, other extracellular matrix proteins, synthetic fragments of any of the proteins, or any combination thereof), by the changes in the shape of the platelets, or any combinations thereof. In one embodiment, platelet activation is measured by changes in the levels of one or more analytes released by platelets. For example, the one or more analytes released by platelets can be P-selectin (CD62p), CD63, ATP, or any combination thereof

In another embodiment, platelet activation is measured by the ability of platelets to adhere to a static surface which is derivatized with one or more relevant ligands. In certain embodiments, the one or more relevant ligands include fibrinogen, von Willebrand factor, collagen, other extracellular matrix proteins, synthetic fragment thereof, or any combination thereof

In other embodiments, platelet activation is measured by the degree of phosphorylation of vasodilator-stimulated phosphoprotein (VASP) upon platelet activation. In yet other embodiments, platelet activation is measured by the level of platelet-leukocyte aggregates. In certain embodiments, platelet activation is measured by proteomics profiling.

In a particular embodiment, platelet activation is measured by the level of binding of fibrinogen to platelets. The fibrinogen binding to platelets can be measured by an anti-fibrinogen antibody, which can be selected from F0111, LS-B2573, LS-B697, LS-B5249, LS-B381, LS-B3048, LS-B7075, LS-052057, LS-C109177, LS-C150799, MCA2760, 4440-8004, NBP1-33582, NB600-926, NBP1-47442, NBP2-11515, NB120-10070, NBP1-96183, NBP1-96180, or any combinations thereof.

In one embodiment, the diagnostic score is measured by F0111, which detects fibrinogen binding to platelets. In another embodiment, the diagnostic score comprises a percentage of platelet activation, which is about 20%, about 25%, about 30%, about 35%, about 40%, about 45%, or about 50% of activated platelets. In other embodiments, the diagnostic score comprises a percentage of platelet activation, which is about 40%, about 45%, about 50%, about 55%, about 60%, about 65%, about 70%, about 75%, about 80%, about 85%, about 90%, or about 95% of activated platelets.

In some embodiments, in a population sample the percentages of platelet activation observed may not follow a normal distribution (See, e.g., FIG. 2). The empirical distribution of the data can be visualized by fitting a logistic normal distribution, a distribution where each data point is scaled from 0% to 100% (See, e.g., FIG. 3). When data are successfully fit to a logistic normal distribution, the original data can be logit transformed to approximately follow a normal distribution, thus allowing statistical analyses with statistical methods based upon data being drawn from a population with a normal distribution. In such embodiments, percentage positive values may be logit transformed, mapping the data from 0% to 100%. These logit transformed data can then be standardized, adjusting the sample mean of each logistic transformed response to zero and the sample variance of each logistic transformed response to one. (See, e.g., FIG. 5). Then these logit-transformed, standardized, data can be adjusted for extraneous factors not biologically related to the percentage of platelet aggregation. The resulting values, or residuals, can be ranked from highest to lowest and their relative position can be used for selection purposes.

In some embodiments, the diagnostic score that normal PAR4 agonist responders demonstrate is equal to or above a normal diagnostic score and below a high diagnostic score. The diagnostic score that high PAR4 agonist responders demonstrate in a population is equal to or above a high diagnostic score. The diagnostic score that the low PAR4 agonist responders demonstrate in a population is below a normal diagnostic score. In certain embodiments, a normal diagnostic score is −0.9, −0.8, −0.7, −0.6, −0.5, −0.4, −0.3, −0.2, or −0.1 of the distribution of platelet activation measurement results observed in a population sample in a scaling from −1 to +1 with 0 being equal to the average. In other embodiments, the normal diagnostic score is 10%, 15%, 20%, 25%, 30%, 35%, or 40% in the distribution of platelet activation measurement results observed in a population sample. In a specific embodiment, the normal diagnostic score is the lower −0.5 (25%) of the distribution of platelet activation measurement results observed in a population sample.

In other embodiments, a high diagnostic score is +0.1, +0.2, +0.3, +0.4, +0.5, +0.6, +0.7, +0.8, or +0.9 of the distribution of platelet activation measurement results observed in a population sample. In certain embodiments, a high diagnostic score is 60%, 65%, 70%, 75%, 80%, 85%, or 90% in the distribution of platelet activation measurement results observed in a population sample. In some examples, a high diagnostic score is +0.5 (75%) of the distribution of platelet activation measurement results observed in a population sample. In some embodiments, the platelet activation measurement results observed in a population sample is FIG. 2B.

In certain embodiments, the diagnostic score is measured by a specific antibody against P-selectin. Examples of the P-selectin antibody include, but are not limited to, CLB-Thromb/6, AK4, 1E3, CTB201, P8G6, MAB2154, ab91132, LS-B3578, LS-B3656, LS-C134593, LS-C13963, 10-667-C100, 11-450-C100, 1A-450-T100, 1Y-450-T100, OASA02343, PN IM1315, or any combinations thereof. In a particular embodiment, the diagnostic score is measured by CLB-Thromb/6. In some embodiments, the normal diagnostic score comprises a percentage of platelet activation, which is about 60%, about 65%, about 70%, about 75%, or about 80% of activated platelets.

In certain embodiments, the invention includes methods for determining if the dose of a PAR4 antagonist is appropriate. Such methods comprise (i) obtaining a blood sample from a subject that has been treated with a PAR4 antagonist, (ii) treating platelets from the blood sample with a PAR4 agonist in vitro, (iii) measuring platelet activation and (iv) comparing the platelet activation in the blood sample following PAR4 antagonist treatment with the platelet activation in a blood sample obtained prior to PAR4 antagonist treatment.

In other embodiments, the invention includes a method for administering a PAR4 antagonist to a subject, the method comprising: (i) obtaining a blood sample from a subject that has been treated with a PAR4 antagonist, (ii) treating platelets from the blood sample with a PAR4 agonist in vitro, (iii) measuring platelet activation, (iv) comparing the platelet activation in the blood sample following the PAR4 antagonist treatment with the platelet activation in a blood sample obtained prior to the PAR4 antagonist treatment, and (v)(a) increasing the subject's dose of the PAR4 antagonist if the subject's sample obtained after the PAR4 antagonist treatment has high reactivity for the PAR4 agonist, or (v)(b) maintaining the subject's dose of the PAR4 antagonist if the subject's sample obtained after the PAR4 antagonist treatment has low reactivity for the PAR4 agonist.

The invention also includes a kit for identifying a subject who is responsive to PAR4 antagonist therapy comprising a PAR4 agonist and an instructional material, which instructs a healthcare provider to mix the PAR4 agonist with platelets in a sample taken from a subject in need of PAR4 antagonist therapy and to measure the platelet activation, wherein the PAR4 agonist comprises an amino acid sequence of Formula I:

Ala-Xₐₐ₁-Pro-Gly-Xₐₐ₂-Leu-Val (Formula I)
wherein,
the amino terminus of the peptide is not fused to an amino acid;
Xₐₐ₁ is selected from Tyr and Phe(4-F);
Xₐₐ₂ is selected from Trp(5-OH), (D,L)-Trp(5-Br), D-Trp, Bzt, Tpi, His, Tza, 3-Thi, 3-Fur, His(Bzl), Phe, Tyr, Phe(penta-F), 2-Pya, 3-Pya, 4-Pya, Dpa, 3-Pya(4-Tolyl), Bip(2-Methyl), 1-Naphthyl-Ala, 2-Naphthyl-Ala, Tyr(Bzl), or Styryl-Ala; and
is a peptide bond; and the peptide has a PAR4 agonist activity.

### BRIEF DESCRIPTION OF THE DRAWINGS/FIGURES

FIG. 1 shows various combinations of a normal diagnostic score (horizontal line) and a high diagnostic score (vertical line), each of which shows a percentile of the distribution of platelet activation measurement results observed in a population sample.

FIG. 2 shows the distribution of platelet responses for each of the four measurements for the 501 subjects studied in Example 4. FIG. 2A shows box plots of the percentage of positive platelets for each agonist and activation marker, showing medians, range and interquartile ranges and outliers. FIG. 2B shows frequency distribution histograms of the same data sets.

FIG. 3 shows plots of curves showing fit of the logistic normal distribution to the distribution of % positive platelet measurements for each agonist-response pair. The area under each curve has been standardised to equal the number of study subjects (501) allowing the comparison of curves.

FIG. 4 is a plot showing the association between the transformed adjusted standardized responses to PAR1 and PAR4 as measured by FIG. 4A) P-selectin expression and FIG. 4B) fibrinogen binding.

FIG. 5 is a plot showing the associations between the transformed adjusted standardized measures of response to agonism by FIG. 5A) PAR1 and FIG. 5B) PAR4.

FIG. 6 is a plot showing the association between the transformed standardised covariate adjusted responses to PAR1 and PAR4.

FIG. 7A-B are plots of individual concentration-response curves for the inhibitor UDM-2555. The percentages of platelets inhibited for (A) P-selectin expression and (B) fibrinogen binding were plotted against the log concentration range of the inhibitor.

FIG. 8 is a plot of concentration-response curves for the inhibitor UDM-2555. The mean percentages of platelets inhibited for (A) P-selectin expression and (B) fibrinogen binding were plotted against the log concentration range of the inhibitor for the 12 individuals tested.

FIG. 9 is a plot showing the P-Selectin and fibrinogen binding response in recall individuals from Part A of the study. Crosses are all subjects from the original test in Part A study. Circles are the hyper responders, triangles, the mid responders and squares the hypo responders recalled for PAR4 inhibitor testing.

FIG. 10 is a schematic showing how blood samples were tested in Part B of Example 4 below.

FIG. 11A is a plot showing the association between the platelet fibrinogen binding response to PAR4 between first and second blood donations. The mean time between the two bleeds in these individuals was 256 days (range=100 to 480 days). R2 value 0.705, P<0.0001. FIG. 11B is a plot showing the association between the platelet P-Selectin expression response to PAR4 between first and second donations. R2 value 0.793, P<0.0001.

FIG. 12A is a plot showing the IC₅₀ of inhibitor of PAR4 response using fibrinogen binding in each group of recall donors. Circles are the hyper responders, triangles, the mid and squares the hypo responders. P values are for an unpaired t test (1 tailed). FIG. 12B is a plot showing the IC₅₀ of inhibitor of PAR4 response using P-selectin in each group of recall donors. Circles are the hyper responders, triangles, the mid and squares the hypo responders. P values are for an unpaired t test (1 tailed).

FIG. 13A is a plot showing the percent positive platelet response to PAR4 versus IC₅₀ using fibrinogen binding in each recall donor. Circles are the hyper responders, triangles, the mid and squares the hypo responders. FIG. 13B is a plot showing the percent positive platelet response to PAR4 versus IC₅₀ using P-Selectin expression in each recall donor. Circles are the hyper responders, triangles, the mid and squares the hypo responders.

FIG. 14 is a plot showing the IC₅₀ values for the inhibitor response in each individual using fibrinogen binding and P-selectin expression. Circles are the hyper responders, triangles, the mid and squares the hypo responders.

### DETAILED DESCRIPTION OF THE INVENTION

The present disclosure provides methods of identifying, diagnosing, treating and monitoring patients for drug treatment options.

### I. DEFINITIONS

As used herein, the singular forms “a”, “an” and “the” include plural referents unless the context clearly dictates otherwise. The terms “a” (or “an”), as well as the terms “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

Wherever embodiments are described herein with the language “comprising,” otherwise analogous embodiments described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or embodiments of the disclosure, which can be had by reference to the specification as a whole. Accordingly, the terms defined immediately below are more fully defined by reference to the specification in its entirety. Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter codes.

An “isolated” polypeptide, antibody, polynucleotide, vector, cell, or composition refers to a polypeptide, antibody, polynucleotide, vector, cell, or composition that is in a form not found in nature. Isolated polypeptides, antibodies, polynucleotides, vectors, cells or compositions include those which have been purified to a degree that they are no longer in a form in which they are found in nature. In some aspects, an antibody, polynucleotide, vector, cell, or composition that is isolated is substantially pure. In some aspects an antibody, polynucleotide, vector, cell, or composition that is isolated is “recombinant.”

The term “antibody” is used herein in its broadest sense and includes, e.g., monoclonal antibodies, polyclonal antibodies, multivalent antibodies, multispecific antibodies, chimeric antibodies, humanized antibodies, human antibodies generated by recombinant technology. The term “antibody” includes whole antibodies. The term “antibody” also refers to a protein comprising at least two immunoglobulin heavy (H) chains and two immunoglobulin light (L) chains inter-connected by disulfide bonds, or an antigen binding portion thereof. Each heavy chain is comprised of a heavy chain variable region (abbreviated herein as VH) and a heavy chain constant region (abbreviated herein as CH). The CH is normally comprised of three domains, CH1, CH2 and CH3 (IgM, e.g., has an additional constant region domain, CH4). Each light chain is comprised of a light chain variable region (abbreviated herein as VL) and a light chain constant region (abbreviated herein as CL). The CL is comprised of one domain and can be of the lambda or kappa type. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In certain embodiments, the VH and VL together comprise a binding domain that interacts with an antigen. In other embodiments a single VH or single VL domain can interact specifically with the antigen. The CH domain of an antibody can mediate the binding of the immunoglobulin to host tissues or factors, including various cells of the immune system (e.g., effector cells), cells lining the vascular wall, other cell expressing receptors for the CH domain of immunoglobulins and the first component (C1q) of the classical complement system.

The term “antigen-binding fragment” refers to one or more fragments of an antibody that retain the ability to specifically bind to an antigen (e.g., fibrinogen or P-selectin). Fragments of a full-length antibody can perform the antigen-binding function of an antibody. Examples of binding fragments encompassed within the term “antigen-binding fragment” of an antibody include (i) a Fab fragment, a monovalent fragment consisting of the VL and CL, VH and CH1 domains; (ii) a F(ab)₂ fragment, a bivalent fragment comprising two Fab fragments linked by a disulfide bridge at the hinge region; (iii) a Fd fragment consisting of the VH and CH1 domains; (iv) a Fv fragment consisting of the VH and CL domains of a single arm of an antibody, (v) a single domain antibody fragment or dAb (Ward et al., (1989) Nature 341:544-546), which consists of a VH domain or a VL domain only; and (vi) an isolated complementarity determining region (CDR). Furthermore, although the two domains of the Fv fragment, VH and VL, are coded for by separate genes, they can be joined, using recombinant or synthetic methods, e.g., by a synthetic linker that enables them to be made as a single protein chain in which the VH and VL regions pair to form monovalent molecules (known as single chain Fv (scFv); see e.g., Bird et al. (1988) Science 242:423-426; and Huston et al. (1988) Proc. Natl. Acad. Sci. USA 85:5879-5883). scFv are also encompassed within the term “antigen-binding fragment” of an antibody. These antibody fragments are obtained using conventional techniques known to those with skill in the art, and the fragments are screened for utility in the same manner as are intact antibodies.

The term “monoclonal antibody” as used herein refers to a preparation of antibody molecules of single molecular composition. A monoclonal antibody displays a single binding specificity and affinity for a particular epitope. The monoclonal antibodies can be generated from any animal, e.g., mouse, rat, rabbit, pig, etc., or can be generated synthetically and be in part or entirely of human sequence.

The term “polyclonal antibody” as used herein refers to a mixture of antibodies purified from the serum of a mammal in which an antigen is injected to generate the antibodies against the antigen. Polyclonal antibodies can be generated from any mammal, e.g., mouse, rat, rabbit, pig, human, etc., or can be generated synthetically, e.g., as a VH and VL gene phage display library.

The term “antibody” as used herein also includes “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al, Proc. Natl. Acad. Sci. USA 81:6851-6855 (1984)).

Basic antibody structures in vertebrate systems are well understood. See, e.g., Harlow et al. (1988) Antibodies: A Laboratory Manual (2nd ed.; Cold Spring Harbor Laboratory Press).

The term “diagnostic score” as used herein can be determined by measuring platelet activation in a sample, e.g., whole blood or platelet rich plasma (PRP), by a PAR4 agonist. In one embodiment, a diagnostic score is a percentile of a subject's platelet activation falling within a distribution curve of platelet activation measurements observed in a population sample. Such a diagnostic score can also be identified using different statistical methods, e.g., an unified and normalized scaling from −1 to +1 with 0 being equal to the average. For example, an individual's platelet activation level can be compared to a distribution curve of platelet activation measurements observed in a population sample. Based on the location where the platelet activation level falls within the distribution curve, the subject can be identified as having below a normal diagnostic score, equal to or above a normal diagnostic score and below a high diagnostic score, or equal to or above a high diagnostic score. In some embodiments, the platelet activation level of platelets from a subject identified as having below a normal diagnostic score falls within the first quartile. In other embodiments, the platelet activation level of platelets from a subject identified as having equal to or above normal diagnostic score and below a high diagnostic score platelet activation level falls within the second and third quartile. In some embodiments, the platelet activation level of platelets from a subject identified as having equal to or above a high diagnostic score falls within the fourth quartile. Platelet activation can be measured by various known methods. Non-limiting examples of such methods are described elsewhere herein.

In another embodiment, a diagnostic score is a percentage of platelet activation (e.g., percentage of positive platelets) measured by a specific antibody, e.g., F0111. Thus, a diagnostic score obtained from platelet activation testing (e.g., 40% platelet activation measured by an anti-fibrinogen antibody, e.g., F0111) can be compared to a pre-determined normal diagnostic score (e.g., 20% platelet activation by an anti-fibrinogen antibody, e.g., F0111) and/or a high diagnostic score (e.g., 50% platelet activation by an anti-fibrinogen antibody, e.g., F0111) to determine the individual's responsiveness to a PAR4 agonist. The percentage of activated platelets following exposure to a PAR4 agonist can be quantified as a percentage of positive platelets detected by fibrinogen binding or antibodies against platelets, e.g., antibody against against P-selectin. The diagnostic score for each subject thus can be categorized into four ranges: 1) no score, e.g. no platelet activation detected; 2) below a normal diagnostic score; 3) equal to or above a normal diagnostic score and below a high diagnostic score; and 4) equal to or above a high diagnostic score.

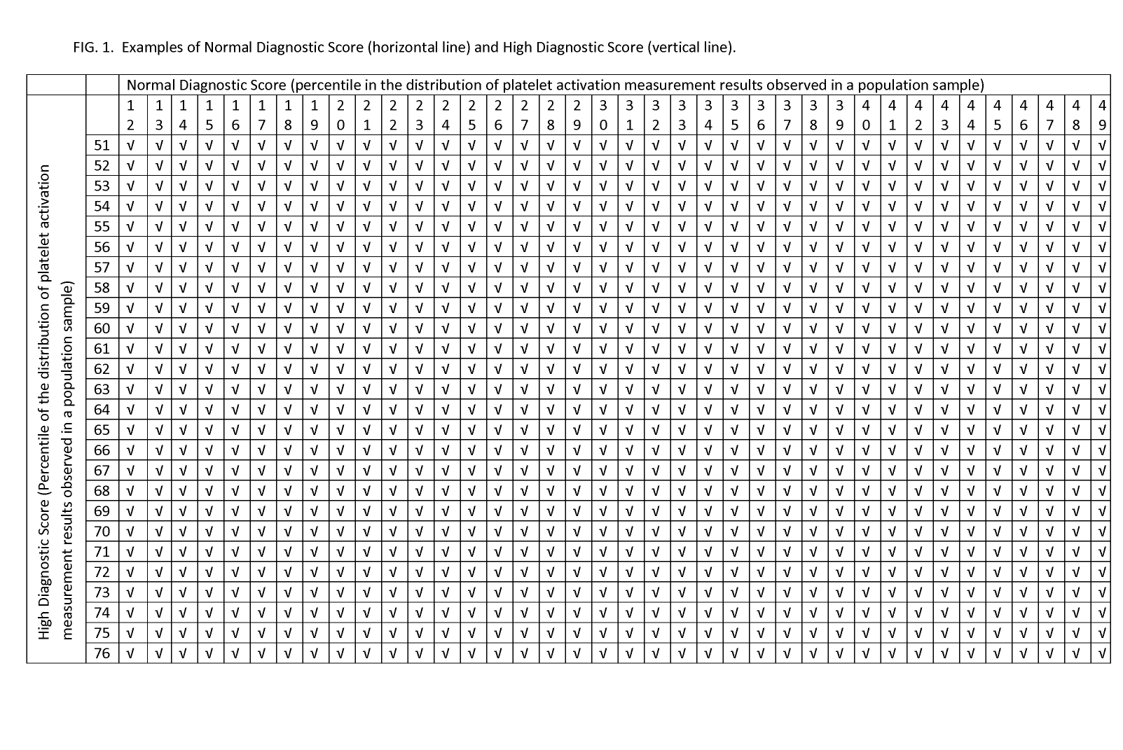

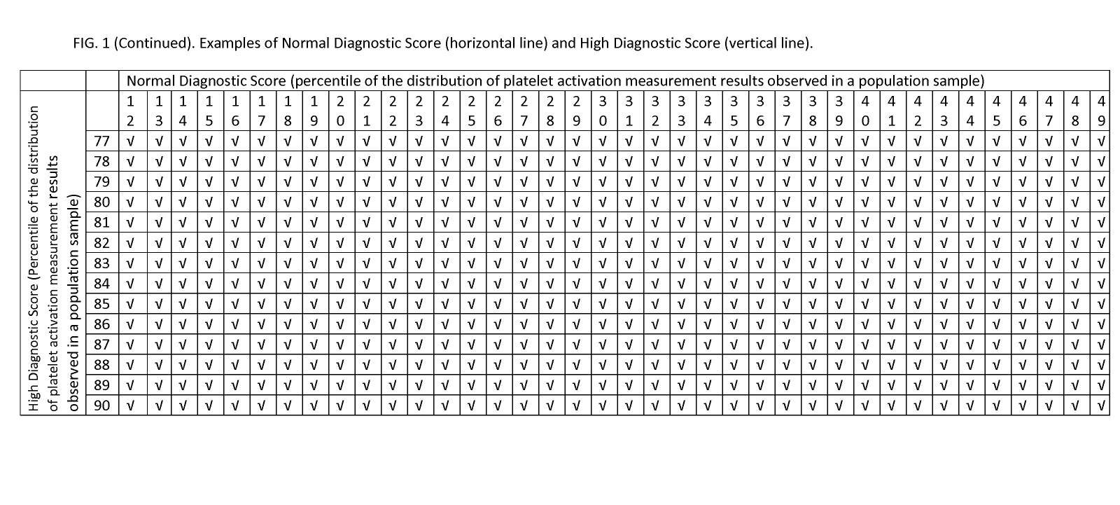

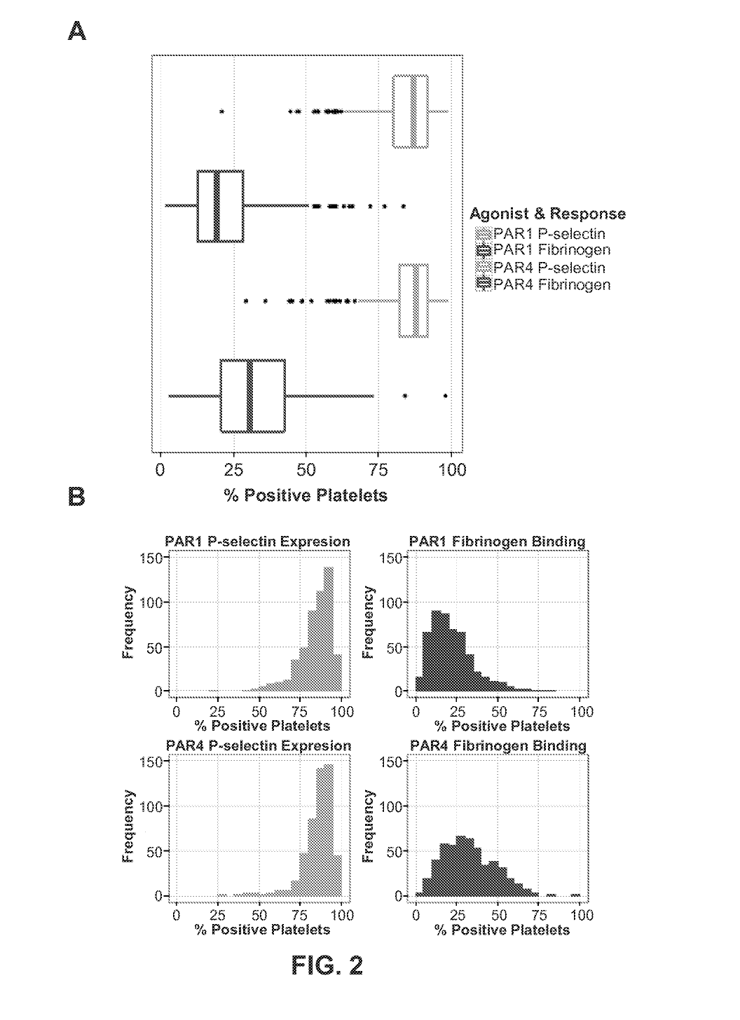

The term “normal diagnostic score” as used herein means the cut-off diagnostic score between low PAR4 agonist responders and normal PAR4 agonist responders of platelet activation in a given population. In one embodiment, the normal diagnostic score is a pre-determined percentile of subjects based on platelet activation which separates normal PAR4 agonist responders from low PAR4 agonist responders within a distribution curve of platelet activation measurements observed in a population sample. In some examples, a normal diagnostic score is any number selected from the horizontal line of FIG. 1, which is indicated as a percentile of a subject's platelet activation falling within a distribution curve of platelet activation measurements observed in a population sample. In other examples, a normal diagnostic score can be indicated as a scale from −1 to 0, 0 being the average. A person of ordinary skill in the art can convert the 0% to 100% scaling to the scaling of −1 to +1. In another embodiment, the normal diagnostic score is 25% or 33.3% of the distribution of measurement results observed in the population sample. In other embodiments, the distribution of measurement results observed in a population sample is FIG. 2B.

In embodiments where platelet activation is measured by fibrinogen binding, a normal diagnostic score can comprise platelet activation of 18%, 19%, 20%, 21%, 22%, 23%, 24%, 25%, 26%, 27%, 28%, 29% or 30% positive platelets. In embodiments where platelet activation is measured by P-selecting expression, a normal diagnostic score can comprise platelet activation of 80%, 81%, 82%, 83%, 84%, 85%, 86%, 87%, 88%, 89%, 90% or 91% positive platelets.

In another embodiment, a normal diagnostic score is a pre-determined percentage of positive platelets measured by a specific antibody. In some embodiments, the normal diagnostic score is based on assay conditions identified by a population study, see, e.g., anti-fibrinogen antibody population study shown in Example 2. Additional agents for measuring platelet activation and/or PAR4 agonists can be calibrated using methods known in the art, see, e.g., Example 3. In some examples, a normal diagnostic score is the median percentage of positive cells minus 10% of positive cells, the median percentage minus 15% of positive cells, the median percentage minus 20% of positive cells, the median percentage minus 25% of positive cells, the median percentage minus 30% of positive cells, the median percentage minus 35% of positive cells, or the median percentage minus 40% of positive cells. In certain examples, a normal diagnostic score is about 10%, about 15%, about 20%, about 25%, about 30%, about 35%, about 40%, or about 45% of positive platelet measured by an anti-fibrinogen antibody.

The term “high diagnostic score” as used herein means the cut-off diagnostic score between the normal PAR4 agonist responders and the high PAR4 agonist responders of platelet activation. In one embodiment, the high diagnostic score is a pre-determined percentile of subjects based on the platelet activation that separates high PAR4 agonist responders from normal PAR4 agonist responders within a distribution curve of platelet activation measurements observed in a population sample. In some examples, the high diagnostic score is any number selected from the vertical line of FIG. 1, which is indicated as a percentile of a subject's platelet activation falling within a distribution curve of platelet activation measurements observed in a population sample. In other examples, the high diagnostic score is 75% or 66.6% of the distribution of measurement results observed in the population sample. In other embodiments, the distribution of measurement results observed in a population sample is FIG. 2B.

In embodiments where platelet activation is measured by fibrinogen binding, a high diagnostic score can comprise platelet activation of 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69% or 70% positive platelets. In embodiments where platelet activation is measured by P-selecting expression, a high diagnostic score can comprise platelet activation of 90%, 91%, 92%, 93%, 94%, 95%, 96%, 97%, 98%, or 99% positive platelets.

In other embodiments, a high diagnostic score is a pre-determined percentage of positive platelets measured by a specific antibody. In some embodiments, the normal diagnostic score is based on assay conditions identified by a population study, see, e.g., anti-fibrinogen antibody population study shown in Example 2. Additional agents for measuring platelet activation and/or PAR4 agonists can be calibrated using methods know in the art, see, e.g., Example 3. In some examples, a high diagnostic score is the median percentage of positive cells plus 10% of positive cells, the median percentage plus 15% of positive cells, the median percentage plus 20% of positive cells, the median percentage plus 25% of positive cells, the median percentage plus 30% of positive cells, the median percentage plus 35% of positive cells, or the median percentage plus 40% of positive cells. In certain examples, a high diagnostic score is about 55%, about 60%, about 65%, about 70%, about 75%, about 80%, about 85%, or about 90% of positive platelet measured by an anti-fibrinogen antibody.

The term “normal responder” as used herein can be interchangeably used with “mid responder” and refers to a subject who demonstrates platelet activation by a PAR4 agonist equal to or above a normal diagnostic score and below a high diagnostic score. Normal responders to a PAR4 agonist are candidates for treatment with PAR4 antagonist. The term “high responder” as used herein can be interchangeably used with “hyper responder” and refers to a subject who demonstrates platelet activation by a PAR4 agonist above a high diagnostic score, which in some embodiments is in the upper quartile of the results observed in a appropriate sized population sample. High responders to the PAR4 agonist can be optimal candidates for treatment with PAR4 antagonist. The term “low responder” as used herein can be interchangeably used with “hypo responder” and refers to a subject who demonstrates platelet activation by a PAR4 agonist below a normal diagnostic score, which in some embodiments is in the lower quartile of the distribution of results observed in a sample of subjects drawn from the relevant population. Low responders to the PAR4 agonist can optinally be treated with PAR4 antagonist. The term “non-responder” as used herein refers to a subject who demonstrates undetectable or vey low platelet activation by a PAR4 antagonist. A non-responder is typically not considered a candidate for PAR4 treatment. A summary of the diagnostic score patient stratification for treatment with PAR4 antagonist is shown in Table 1.

TABLE 1A
Categorization of Responders based on Platelet
Activation - No PAR4 Antagonist pre-treatment

| | Response to | PAR4 Antagonist |
|---|---|---|
| Diagnostic Score | PAR4 agonist | Treatment |
| No Response - No | No response | Do not treat with PAR4 |
| Diagnostic Score | | antagonist |
| Lower than Normal | Low | Optionally do not treat with |
| Diagnostic Score | | PAR4 antagonist |
| Equal to or above Normal | Normal | Treat with PAR4 antagonist |
| Diagnostic Score and below | | |
| High Diagnostic Score | | |
| Equal to or above High | High | Treat with PAR4 antagonist |
| Diagnostic Score | | |

TABLE 1B
Categorization of Responders based on Platelet
Activation - PAR4 Antagonist pre-treatment

| | PAR4 Antagonist Dose Adjustment |
|---|---|
| PAR4 agonist Inhibition | Strategy |
| High Percentage Inhibition | No dose adjustmnet |
| Low Percentage Inhibition | Dose adjustment recommended |

As used herein, the terms “treat” or “treatment” refer to both therapeutic treatment and prophylactic or preventative measures, wherein the object is to prevent or slow down (lessen) an undesired physiological change or disorder, such as the progression of an thromboembolism, e.g., acute coronary syndromes. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

As used herein, “prophylaxis” or “prevention” refers to the preventive treatment of a subclinical disease-state in a mammal, particularly in a human, aimed at reducing the probability of the occurrence of a clinical disease-state. Patients are selected for preventative therapy based on factors that are known to increase risk of suffering a clinical disease state compared to the general population. “Prophylaxis” therapies can be divided into (a) primary prevention and (b) secondary prevention. Primary prevention is defined as treatment in a subject that has not yet presented with a clinical disease state, whereas secondary prevention is defined as preventing a second occurrence of the same or similar clinical disease state.

The terms “effective amount,” “amount effective to,” “therapeutically effective amount,” “effective dose,” or “therapeutically effective dose,” includes reference to a dosage of a therapeutic agent (e.g., PAR4 antagonist) sufficient to produce a desired result. An effective amount of a PAR4 agonist for each group of patients (i.e., low responder, high responder, and normal responder to PAR4 agonist) can differ based on the platelet activation response by a PAR4 agonist. The effective amount of a PAR4 antagonist for a normal responder is also referred to herein as “a standard effective amount.” An effective amount of a PAR4 antagonist for a low PAR4 agonist responder can be lower than the standard effective amount. An effective amount of a PAR4 antagonist for a high PAR4 agonist responder can be higher than the standard effective amount.

The term “PAR4 antagonist therapy” as used herein means treatment of a subject with a PAR4 antagonist.

By “subject” or “patient” is meant any subject, particularly a mammalian subject, for whom diagnosis, prognosis, or therapy is desired. As used herein, the terms “subject” or “patient” include any human or nonhuman animal. The term “nonhuman animal” includes all vertebrates, e.g., mammals and non-mammals, such as nonhuman primates, sheep, dogs, cats, horses, cows, bears, chickens, amphibians, reptiles, etc. As used herein, phrases such as “a subject having a disease or condition associated with thromboembolism” includes subjects, such as mammalian subjects, that would benefit from the administration of a PAR4 antagonist.

The term “free” is a term that is well known to those of skill in the art. For example, the term “free” can be used to mean that the amino terminus of the peptide is not blocked or modified. The term “free” can also be used to mean that the amino terminus of the peptide is not fused to an amino acid.

The hyphen (−) in Formulas (I) to (IV) and as used in amino acid sequences herein, represents a peptide bond.

As used herein, the term “alkyl” or “alkylene”, alone or as part of another group, is intended to include both branched and straight-chain saturated aliphatic hydrocarbon groups having from 1 to 10 carbons or the specified number of carbon atoms. For example, “C₁₋₁₀ alkyl” (or alkylene), is intended to include C₁, C₂, C₃, C₄, C₅, C₆, C₇, C₈, C₉, and C₁₀ alkyl groups. Additionally, for example, “C₁-C₆ alkyl” denotes alkyl having 1 to 6 carbon atoms. Alkyl groups can be unsubstituted or substituted with at least one hydrogen being replaced by another chemical group. Example alkyl groups include, but are not limited to, methyl (Me), ethyl (Et), propyl (e.g., n-propyl and isopropyl), butyl (e.g., n-butyl, isobutyl, t-butyl), and pentyl (e.g., n-pentyl, isopentyl, neopentyl), as well as chain isomers thereof, and the like as well as such groups which can optionally include 1 to 4 substituents such as halo, for example F, Br, Cl, or I, or CF₃, alkyl, alkoxy, aryl, aryloxy, aryl(aryl) or diaryl, arylalkyl, arylalkyloxy, alkenyl, cycloalkyl, cycloalkylalkyl, cycloalkylalkyloxy, amino, hydroxy, hydroxyalkyl, acyl, heteroaryl, heteroaryloxy, heteroarylalkyl, heteroarylalkoxy, aryloxyalkyl, alkylthio, arylalkylthio, aryloxyaryl, alkylamido, alkanoylamino, arylcarbonylamino, nitro, cyano, thiol, haloalkyl, trihaloalkyl, and/or alkylthio as well as (═O), ORₐ, SRₐ, (═S), —NRₐR_{b}, —N(alkyl)₃⁺, —NRₐSO₂, —NRₐSO₂R_{c}, —SO₂R_{c}—SO₂NRₐR_{b}, —SO₂NRₐC(═O)R_{b}, SO₃H, —PO(OH)₂, —C(═O)Rₐ, —CO₂Rₐ, —C(═O)NRₐR_{b}, —C(═O)(C₁-C₄ alkylene)NRₐR_{b}, —C(═O)NRₐ(SO₂)R_{b}, —OO₂(C₁-C₄ alkylene)NRₐR_{b}, —NRₐC(═O)R_{b}, —NRₐCO₂R_{b}, —NRₐ(C₁-C₄ alkylene)CO₂R_{b}, ═N—OH, ═N—O-alkyl, wherein Rₐ and R_{b} are the same or different and are independently selected from hydrogen, alkyl, alkenyl, CO₂H, CO₂(alkyl), C₃-C₇cycloalkyl, phenyl, benzyl, phenylethyl, naphthyl, a 4- to 7-membered heterocyclo, or a 5- to 6-membered heteroaryl, or when attached to the same nitrogen atom may join to form a heterocyclo or heteroaryl, and R_{c} is selected from same groups as Rₐ and R_{b} but is not hydrogen. Each group Rₐ and R_{b} when other than hydrogen, and each R_{c} group optionally has up to three further substituents attached at any available carbon or nitrogen atom of Rₐ, R_{b}, and/or R_{c}, said substituent(s) being the same or different and are independently selected from the group consisting of (C₁-C₆)alkyl, (C₂-C₆)alkenyl, hydroxy, halogen, cyano, nitro, CF₃, O(C₁-C₆ alkyl), OCF₃, C(═O)H, C(═O)(C₁-C₆ alkyl), CO₂H, CO₂(C₁-C₆ alkyl), NHCO₂(C₁-C₆ alkyl), —S(C₁-C₆ alkyl), —NH₂, NH(C₁-C₆ alkyl), N(C₁-C₆ alkyl)₂, N(CH₃)₃⁺, SO₂(C₁-C₆ alkyl), C(═O)(C₁-C₄ alkylene)NH₂, C(═O)(C₁-C₄ alkylene)NH(alkyl), C(═O)(C₁-C₄ alkylene)N(C₁-C₄ alkyl)₂, C₃-C₇ cycloalkyl, phenyl, benzyl, phenylethyl, phenyloxy, benzyloxy, naphthyl, a 4- to 7-membered heterocyclo, or a 5- to 6-membered heteroaryl. When a substituted alkyl is substituted with an aryl, heterocyclo, cycloalkyl, or heteroaryl group, said ringed systems are as defined below and thus can have zero, one, two, or three substituents, also as defined below.

“Alkenyl” or “alkenylene”, alone or as part of another group, is intended to include hydrocarbon chains of either straight or branched configuration and having one or more carbon-carbon double bonds that can occur in any stable point along the chain. For example, “C₂₋₆ alkenyl” (or alkenylene), is intended to include C₂, C₃, C₄, C₅, and C₆ alkenyl groups. Examples of alkenyl include, but are not limited to, ethenyl, 1-propenyl, 2-propenyl, 2-butenyl, 3-butenyl, 2-pentenyl, 3-pentenyl, 4-pentenyl, 2-hexenyl, 3-hexenyl, 4-hexenyl, 5-hexenyl, 2-methyl-2-propenyl, and 4-methyl-3-pentenyl, and which can be optionally substituted with 1 to 4 substituents, namely, halogen, haloalkyl, alkyl, alkoxy, alkenyl, alkynyl, aryl, arylalkyl, cycloalkyl, amino, hydroxy, heteroaryl, cycloheteroalkyl, alkanoylamino, alkylamido, arylcarbonyl-amino, nitro, cyano, thiol, and/or alkylthio.

“Alkynyl” or “alkynylene”, alone or as part of another group, is intended to include hydrocarbon chains of either straight or branched configuration and having one or more carbon-carbon triple bonds that can occur in any stable point along the chain. For example, “C₂₋₆ alkynyl” (or alkynylene), is intended to include C₂, C₃, C₄, C₅, and C₆ alkynyl groups; such as ethynyl, propynyl, butynyl, pentynyl, and hexynyl, and which can be optionally substituted with 1 to 4 substituents, namely, halogen, haloalkyl, alkyl, alkoxy, alkenyl, alkynyl, aryl, arylalkyl, cycloalkyl, amino, heteroaryl, cycloheteroalkyl, hydroxy, alkanoylamino, alkylamido, arylcarbonylamino, nitro, cyano, thiol, and/or alkylthio.

The term “alkoxy” or “alkyloxy”, alone or as part of another group, refers to an —O-alkyl group, where alkyl is as defined above. “C₁₋₆ alkoxy” (or alkyloxy), is intended to include C₁, C₂, C₃, C₄, C₅, and C₆ alkoxy groups. Example alkoxy groups include, but are not limited to, methoxy, ethoxy, propoxy (e.g., n-propoxy and isopropoxy), and t-butoxy. Similarly, “alkylthio” or “thioalkoxy”, alone or as part of another group, represents an alkyl group or alkoxy group as defined above with the indicated number of carbon atoms attached through a sulphur bridge; for example methyl-S— and ethyl-S—.

“Halo” or “halogen”, alone or as part of another group, includes fluoro, chloro, bromo, and iodo.

“Haloalkyl” is intended to include both branched and straight-chain saturated aliphatic hydrocarbon groups having the specified number of carbon atoms, substituted with 1 to 7 halogens, preferably 1 to 4 halogens, preferably F and/or Cl. Examples of haloalkyl include, but are not limited to, fluoromethyl, difluoromethyl, trifluoromethyl, trichloromethyl, pentafluoroethyl, pentachloroethyl, 1,1-difluoroethyl, 1-fluoroethyl, 2,2,2-trifluoroethyl, heptafluoropropyl, and heptachloropropyl. Examples of haloalkyl also include “fluoroalkyl” that is intended to include both branched and straight-chain saturated aliphatic hydrocarbon groups having the specified number of carbon atoms, substituted with 1 to 7 fluorine atoms, preferably 1 to 4 fluorine atoms.

“Halo-C₁-C₂-alkoxy” or “haloalkyloxy” represents a haloalkyl group as defined above with the indicated number of carbon atoms attached through an oxygen bridge. For example, “C₁₋₆ haloalkoxy”, is intended to include C₁, C₂, C₃, C₄, C₅, and C₆ haloalkoxy groups. Examples of haloalkoxy include, but are not limited to, trifluoromethoxy, 2,2,2-trifluoroethoxy, pentafluorothoxy, and the like. Similarly, “haloalkylthio” or “thiohaloalkoxy” represents a haloalkyl group as defined above with the indicated number of carbon atoms attached through a sulphur bridge; for example trifluoromethyl-S—, and pentafluoroethyl-S—.

Unless otherwise indicated, the term “cycloalkyl” as employed herein alone or as part of another group includes saturated or partially unsaturated (containing 1 or 2 double bonds) cyclic hydrocarbon groups containing 1 to 3 rings, including monocyclic alkyl, bicyclic alkyl (or bicycloalkyl), and tricyclic alkyl, containing a total of 3 to 10 carbons forming the ring (C₃-C₁ cycloalkyl), and which can be fused to 1 or 2 aromatic rings as described for aryl, which includes cyclopropyl, cyclobutyl, cyclopentyl, cyclohexyl, cycloheptyl, cyclooctyl, cyclodecyl, cyclododecyl, cyclohexenyl, norbornyl,

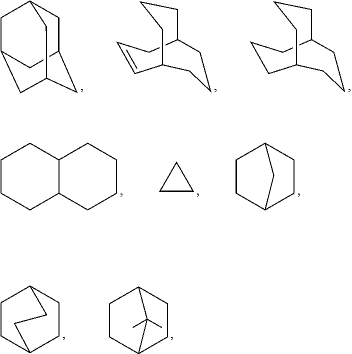

any of which groups can be optionally substituted with 1 to 4 substituents such as halogen, alkyl, alkoxy, hydroxy, aryl, aryloxy, arylalkyl, cycloalkyl, alkylamido, alkanoylamino, oxo, acyl, arylcarbonylamino, amino, nitro, cyano, thiol, and/or alkylthio, and/or any of the substituents for alkyl, as well as such groups including 2 free bonds and thus are linking groups.

As used herein, “carbocycle” or “carbocyclic residue” is intended to mean any stable 3-, 4-, 5-, 6-, or 7-membered monocyclic or bicyclic or 7-, 8-, 9-, 10-, 11-, 12-, or 13-membered bicyclic or tricyclic ring, any of which can be saturated, partially unsaturated, unsaturated or aromatic. Examples of such carbocycles include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, [3.3.0]bicyclooctane, [4.3.0]bicyclononane, [4.4.0]bicyclodecane, [2.2.2]bicyclooctane, fluorenyl, phenyl, naphthyl, indanyl, adamantyl, anthracenyl, and tetrahydronaphthyl (tetralin). As shown above, bridged rings are also included in the definition of carbocycle (e.g., [2.2.2]bicyclooctane). Preferred carbocycles, unless otherwise specified, are cyclopropyl, cyclobutyl, cyclopentyl, cyclohexyl, phenyl, and indanyl. When the term “carbocycle” is used, it is intended to include “aryl”. A bridged ring occurs when one or more carbon atoms link two non-adjacent carbon atoms. Preferred bridges are one or two carbon atoms. It is noted that a bridge always converts a monocyclic ring into a tricyclic ring. When a ring is bridged, the substituents recited for the ring can also be present on the bridge.

“Aryl” groups refer to monocyclic or polycyclic aromatic hydrocarbons, including, for example, phenyl, naphthyl, and phenanthranyl. Aryl moieties are well known and described, for example, in Lewis, R. J., ed., Hawley's Condensed Chemical Dictionary, 13th Edition, John Wiley & Sons, Inc., New York (1997). “C₆₋₁₀ aryl” refers to phenyl and naphthyl. Unless otherwise specified, “aryl”, “C₆₋₁₀ aryl” or “aromatic residue” can be unsubstituted or substituted with 1 to 3 groups selected from OH, OC₁-C₃ alkoxy, Cl, F, Br, I, CN, NO₂, NH₂, N(CH₃)H, N(CH₃)₂, CF₃, OCF₃, OCHF₂, C(═O)CH₃, SCH₃, S(═O)CH₃, S(═O)₂CH₃, C₁-C₃ alkyl, CO₂H, and CO₂CH₃.

As used herein, the term “heterocycle”, “heterocyclo” or “heterocyclic” group is intended to mean a stable 4- to 14-membered monocyclic, bicyclic or tricyclic heterocyclic ring which is saturated or partially unsaturated and which consists of carbon atoms and 1, 2, 3, or 4 heteroatoms independently selected from the group consisting of N, NH, O and S and including any bicyclic group in which any of the above-defined heterocyclic rings is fused to a benzene ring. The nitrogen and sulfur heteroatoms can optionally be oxidized (i.e., N→O and S(O)ₚ, wherein p is 0, 1 or 2). The nitrogen atom can be substituted or unsubstituted (i.e., N or NR wherein R is H or another substituent, if defined). The heterocyclic ring can be attached to its pendant group at any heteroatom or carbon atom that results in a stable structure. The heterocyclic rings described herein can optionally be substituted on carbon or on a nitrogen atom if the resulting compound is stable, with 1 to 3 groups selected from OH, OC₁-C₃ alkoxy, Cl, F, Br, I, CN, NO₂, NH₂, N(CH₃)H, N(CH₃)₂, CF₃, OCF₃, OCHF₂, ═O, C(═O)CH₃, SCH₃, S(═O)CH₃, S(═O)₂CH₃, C₁-C₃ alkyl, CO₂H and CO₂CH₃. A nitrogen in the heterocycle can optionally be quaternized. It is preferred that when the total number of S and O atoms in the heterocycle exceeds 1, then these heteroatoms are not adjacent to one another. It is preferred that the total number of S and O atoms in the heterocycle is not more than 1. Spiro and bridged rings are also included in the definition of heterocycle. A bridged ring occurs when one or more atoms (i.e., C, O, N, or S) link two non-adjacent carbon or nitrogen atoms. Examples of bridged rings include, but are not limited to, one carbon atom, two carbon atoms, one nitrogen atom, two nitrogen atoms, and a carbon-nitrogen group. It is noted that a bridge always converts a monocyclic ring into a tricyclic ring. When a ring is bridged, the substituents recited for the ring can also be present on the bridge. When the term “heterocycle” is used, it is not intended to include heteroaryl.

Exemplary monocyclic heterocyclic groups include azetidinyl, pyrrolidinyl, oxetanyl, imidazolinyl, oxazolidinyl, isoxazolinyl, thiazolidinyl, isothiazolidinyl, tetrahydrofuranyl, piperidyl, piperazinyl, 2-oxopiperazinyl, 2-oxopiperidyl, 2-oxopyrrolodinyl, 2-oxoazepinyl, azepinyl, 4-piperidonyl, tetrahydropyranyl, morpholinyl, thiamorpholinyl, thiamorpholinyl sulfoxide, thiamorpholinyl sulfone, 1,3-dioxolane, and tetrahydro-1,1-dioxothienyl, and the like.

Exemplary bicyclic heterocyclo groups include quinuclidinyl.

Preferred heterocyclo groups include

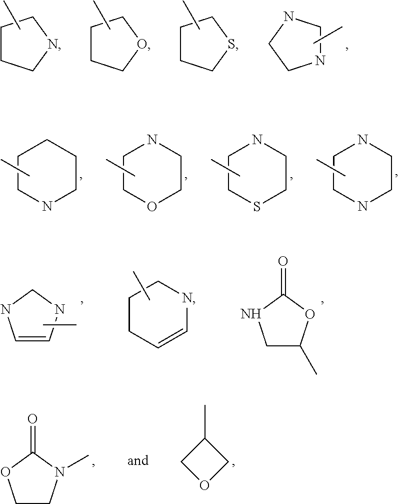

which optionally can be substituted.

As used herein, the term “aromatic heterocyclic group” or “heteroaryl” is intended to mean stable monocyclic and polycyclic aromatic hydrocarbons that include at least one heteroatom ring member such as sulfur, oxygen, or nitrogen. Heteroaryl groups include, without limitation, pyridyl, pyrimidinyl, pyrazinyl, pyridazinyl, triazinyl, furyl, quinolyl, isoquinolyl, thienyl, imidazolyl, thiazolyl, indolyl, pyrroyl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, purinyl, carbazolyl, benzimidazolyl, indolinyl, benzodioxolanyl, and benzodioxane. Heteroaryl groups are unsubstituted or substituted with 1 to 3 groups selected from OH, OC₁-C₃ alkoxy, Cl, F, Br, I, CN, NO₂, NH₂, N(CH₃)H, N(CH₃)₂, CF₃, OCF₃, OCHF₂, ═O, C(═O)CH₃, SCH₃, S(═O)CH₃, S(═O)₂CH₃, C₁-C₃ alkyl, CO₂H and CO₂CH₃. The nitrogen atom is substituted or unsubstituted (i.e., N or NR wherein R is H or another substituent, if defined). The nitrogen and sulfur heteroatoms can optionally be oxidized (i.e., N→O and S(O)ₚ, wherein p is 0, 1 or 2). Bridged rings are also included in the definition of heteroaryl. A bridged ring occurs when one or more atoms (i.e., C, O, N, or S) link two non-adjacent carbon or nitrogen atoms. Examples of bridged rings include, but are not limited to, one carbon atom, two carbon atoms, one nitrogen atom, two nitrogen atoms, and a carbon-nitrogen group. It is noted that a bridge always converts a monocyclic ring into a tricyclic ring. When a ring is bridged, the substituents recited for the ring can also be present on the bridge.

Preferred heteroaryl groups include

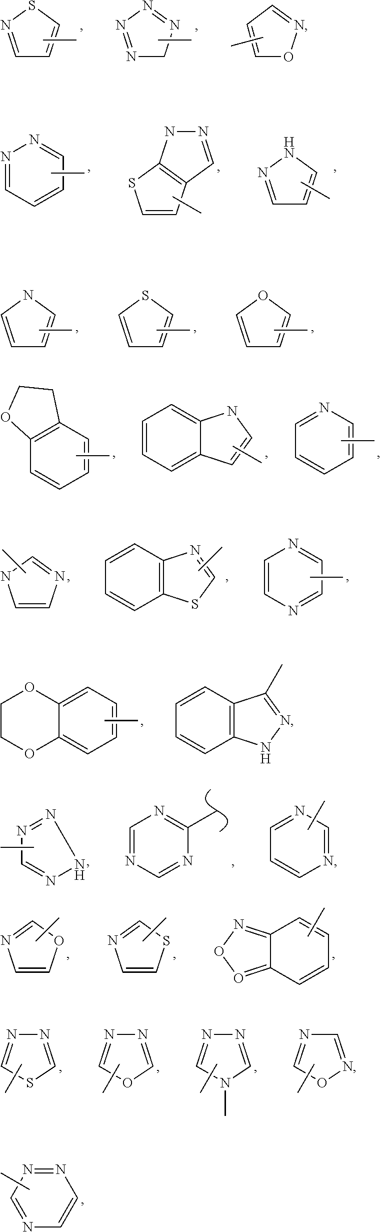

and the like.

When the term “unsaturated” is used herein to refer to a ring or group, which group can be fully unsaturated or partially unsaturated.

The term “acyl” alone or as part of another group refers to a carbonyl group linked to an organic radical, more particularly, the group C(═O)Rₑ, as well as the bivalent groups —C(═O)— or —C(═O)Rₑ—, which are linked to organic radicals. The group Rₑ can be selected from alkyl, alkenyl, alkynyl, aminoalkyl, substituted alkyl, substituted alkenyl, or substituted alkynyl, as defined herein, or when appropriate, the corresponding bivalent group, e.g., alkylene, alkenylene, and the like.

The designation “ [symbol] ” or

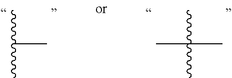

attached to a ring or other group refers to a free bond or linking group.

Throughout the specification, groups and substituents thereof can be chosen by one skilled in the field to provide stable moieties and compounds and compounds useful as pharmaceutically-acceptable compounds and/or intermediate compounds useful in making pharmaceutically-acceptable compounds.

The term “counterion” is used to represent a negatively charged species such as chloride, bromide, hydroxide, acetate, and sulfate.

As referred to herein, the term “substituted” means that at least one hydrogen atom is replaced with a non-hydrogen group, provided that normal valencies are maintained and that the substitution results in a stable compound. When a substituent is keto (i.e., ═O), then 2 hydrogens on the atom are replaced. Keto substituents are not present on aromatic moieties. Ring double bonds, as used herein, are double bonds that are formed between two adjacent ring atoms (e.g., C═C, C═N, or N═N).

In cases wherein there are nitrogen atoms (e.g., amines) on compounds of the present invention, these can be converted to N-oxides by treatment with an oxidizing agent (e.g., mCPBA and/or hydrogen peroxides) to afford other compounds of this invention. Thus, shown and claimed nitrogen atoms are considered to cover both the shown nitrogen and its N-oxide (N→O) derivative. In cases in which there are quaternary carbon atoms in compounds of the present invention, these can be replaced by silicon atoms, provided they do not form Si—N or Si—O bonds.

When any variable occurs more than one time in any constituent or formula for a compound, its definition at each occurrence is independent of its definition at every other occurrence. Thus, for example, if a group is shown to be substituted with 0 to 3 R^{3a}, then said group can optionally be substituted with up to three R^{3a} groups, and at each occurrence R^{3a} is selected independently from the definition of R^{3a}. Also, combinations of substituents and/or variables are permissible only if such combinations result in stable compounds.

When a bond to a substituent is shown to cross a bond connecting two atoms in a ring, then such substituent can be bonded to any atom on the ring. When a substituent is listed without indicating the atom in which such substituent is bonded to the rest of the compound of a given formula, then such substituent can be bonded via any atom in such substituent. Combinations of substituents and/or variables are permissible only if such combinations result in stable compounds.

The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, and/or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Allen, L. V., Jr., ed., Remington: The Science and Practice of Pharmacy, 22nd Edition, Pharmaceutical Press, London, UK (2012), the disclosure of which is hereby incorporated by reference.

In addition, compounds of formula V can have prodrug forms. Any compound that will be converted in vivo to provide the bioactive agent (i.e., a compound of formula V) is a prodrug within the scope and spirit of the invention. Various forms of prodrugs are well known in the art. For examples of such prodrug derivatives, see:
a) Bundgaard, H., ed., Design of Prodrugs, Elsevier (1985), and Widder, K. et al., eds., Methods in Enzymology, 112:309-396, Academic Press (1985);
b) Bundgaard, H., Chapter 5, “Design and Application of Prodrugs”, Krosgaard-Larsen, P. et al., eds., A Textbook of Drug Design and Development, pp. 113-191, Harwood Academic Publishers (1991);
c) Bundgaard, H., Adv. Drug Deliv. Rev., 8:1-38 (1992);
d) Bundgaard, H. et al., J. Pharm. Sci., 77:285 (1988);
e) Kakeya, N. et al., Chem. Pharm. Bull., 32:692 (1984); and
f) Rautio, J (Editor). Prodrugs and Targeted Delivery (Methods and Principles in Medicinal Chemistry), Vol 47, Wiley-VCH, 2011.

Preparation of prodrugs is well known in the art and described in, for example, King, F. D., ed., Medicinal Chemistry: Principles and Practice, The Royal Society of Chemistry, Cambridge, UK (2nd Edition, reproduced (2006)); Testa, B. et al., Hydrolysis in Drug and Prodrug Metabolism. Chemistry, Biochemistry and Enzymology, VCHA and Wiley-VCH, Zurich, Switzerland (2003); Wermuth, C. G., ed., The Practice of Medicinal Chemistry, 3rd Edition, Academic Press, San Diego, Calif. (2008).

The term “solvate” means a physical association of a compound of this invention with one or more solvent molecules, whether organic or inorganic. This physical association includes hydrogen bonding. In certain instances the solvate will be capable of isolation, for example when one or more solvent molecules are incorporated in the crystal lattice of the crystalline solid. “Solvate” encompasses both solution-phase and isolable solvates. Exemplary solvates include, but are not limited to, hydrates, ethanolates, methanolates, and isopropanolates. Methods of solvation are generally known in the art.

The term “thrombosis,” as used herein, refers to formation or presence of a thrombus (pl. thrombi) within a blood vessel that can cause ischemia or infarction of tissues supplied by the vessel. The term “embolism,” as used herein, refers to sudden blocking of an artery by a clot or foreign material that has been brought to its site of lodgment by the blood current. The term “thromboembolism,” as used herein, refers to obstruction of a blood vessel with thrombotic material carried by the blood stream from the site of origin to plug another vessel. The term “thromboembolic disorders” entails both “thrombotic” and “embolic” disorders (defined above).

The term “pharmaceutical composition,” as used herein means any composition, which contains at least one therapeutically or biologically active agent and is suitable for administration to the patient. For example, a pharmaceutical composition can comprise a PAR4 antagonist and at least one pharmaceutically acceptable carrier. Any of these formulations can be prepared by well-known and accepted methods of the art. See, for example, Gennaro, A. R., ed., Remington: The Science and Practice of Pharmacy, 20th Edition, Mack Publishing Co., Easton, Pa. (2000).

### II. DIAGNOSTIC METHODS AND METHODS OF TREATMENT

Activation of human platelets by thrombin is mediated predominantly through two proteinase-activated receptors (PARs), i.e., PAR1 and PAR4, which belong to the G protein-coupled receptor family. PAR1 and PAR4 differ in the timing and magnitude of signaling. PAR1 triggers a rapid and transient increase in intracellular calcium while PAR4 triggers a slower but more prolonged response. See Shapiro et al., J. Biol. Chem. 275: 25216-25221 (2000). The differences in the kinetics of the signals mediated by PAR1 and PAR4 imply that the two PARs can play distinct roles in the early and late events of platelet activation. For example, studies suggest that PAR1 can account for the initial platelet aggregation in response to thrombin, while PAR4 can contribute to the stability of platelet aggregation. See Covic et al, Nat. Med. 8: 1161-1165 (2002).

The present invention is therefore derived from the recognition that a subject who responds well to a PAR4 agonist (e.g., a subject with platelet activation equal to or above a high diagnostic score who shows a high level of platelet activation by a PAR4 agonist) can be a more suitable candidate for treatment with PAR4 antagonist than a subject with a low diagnostic score. Whereas a subject who responds poorly to a PAR4 agonist (e.g., a subject with platelet activation below a normal diagnostic score who shows a low level of platelet activation by a PAR4 agonist) can not be as suitable of a candidate for treatment with PAR4 antagonist than a subject with a normal diagnostic score. However, it can still be desirable to treat a subject who responds poorly to PAR4 agonist with PAR4 antagonist to determine the clinical outcome in that subject. In one aspect, the invention is directed to a method for identifying a PAR4 agonist responder by an in vitro test using a PAR4 agonist. In another aspect, the invention is directed to a method for identifying a PAR4 antagonist responder by identifying a PAR4 agonist responder.

In one embodiment, the invention includes a method of categorizing a subject as high responders, normal responders, low responders or non-responders to PAR4 agonist. Response to PAR4 agonist can be measured as described herein or as is well known in the art. In some embodiments, measurement of a subject's response to PAR4 agonist comprises (i) obtaining a blood sample from a subject that has not been treated with PAR4 antagonist, (ii) treating platelets from the blood sample with PAR4 agonist in vitro, (iii) measuring platelet activation and (iv) categorizing the subject based on the amount of platelet activation observed.

In certain embodiments, subjects are categorized as low responders, normal responders or high responders using statistical analysis methods known in the art. An example of statistical methods known in the art that can be used for categorizing subjects can be found in Jones et al., Mapping the platelet profile for functional genomic studies and demonstration of the effect size of the GP6 locus, J. Thrombosis and Haemostasis, 5:1756-1765 (2007), which is incorporated by reference herein. In some embodiments, subjects are categorized by the following STATISTICAL PROCEDURE 1.

### 1. Statistical Procedure 1

Step 1: Conduct two flow cytometric whole blood platelet assays in healthy human volunteers, specifically, fibrinogen binding (F) and P-selectin expression (P) platelet responses to a PAR4 agonist. Record the two responses for each subject as a percentage of positive platelets (0 to 100%).

### 1.1 Statistical Refinement

Step 2: Apply a logistic transformation to each of the two sets of percentages in Step 1, with the goal of generating two sets of approximately normally distributed sample data, ≈N(y̅_{F}, s_{F}²) and ≈N(y̅_{P}, s_{P}²), or sample data that are at least less skewed and possibly symmetric.

Step 3: With the goal of standardizing each of the two sets of logistically transformed data in Step 2 to approximately N(0,1), for each data value, subtract the corresponding sample mean (i.e., either y̅_{F} or y̅_{P}) and divide by the corresponding sample standard deviation (i.e., either s_{F} or s_{P}).

Step 4: Through multiple linear regression, adjust each of the two sets of logistic transformed, standardized, responses in Step 3 for experimental and other variables that may have influenced the responses but are not believed to be intrinsically important biologically in determining each individual's response (e.g., sub-study, number of days since study initiation, time of day each sample was agonized). Do not adjust for variables which may be intrinsically biologically related (e.g., gender). Move forward to the selection process the two sets of residuals from the two multiple linear regression analyses.

For this procedure, as outlined in Steps 1 to 4, at this point each subject started with two PAR4 agonist responses (0 to 100%) which were statistically refined to residuals (−infinity to +infinity) and should contain mostly important, but unknown, intrinsic biological information directly related to either hyper- or hypo-response. 1.2 Selection Process

Step 5a (high responders): To select high responders, rank the subjects decreasingly (largest-to-smallest) by the minimum (lesser) of their two refined PAR4 fibrinogen binding and P-selectin expression values (i.e., residuals). Select the upper XX percentile of this ranking, where XX is percentile chosen depending on the variation in the subject population and the stratification desired. In some embodiments, the upper percentile comprises subjects that rank above 65%, 70%, 75%, 80%, 85%, 90%, 95%, 96%, 97%, 98% or 99% of the remaining subjects in the study.

Step 5b (low responders): To select low responders, rank the subjects increasingly (smallest-to-largest) by the maximum (greater) of their two refined PAR4 fibrinogen binding and P-selectin expression values (i.e., residuals). Select the lower XX percentile of this ranking, where XX is percentile chosen depending on the variation in the subject population and the stratification desired. In some embodiments, the lower percentile comprises subjects that rank below 40%, 35%, 30%, 25%, 20%, 15% 10%, 5%, 4%, 3%, 2% or 1% of the remaining subjects in the study.

Step 5c (normal responders): To select the normal responders, rank the subjects increasingly (smallest-to-largest) by the sum of their two absolute deviations from the mean of their two refined PAR4 fibrinogen binding and P-selectin expression values (i.e., residuals). Normal responders will usually be classified as those individuals who fall in a higher percentile than the percentile for the low responders and a lower percentile than the percentile for the high responders.

In some embodiments, subjects are categorized by the following STATISTICAL PROCEDURE 2.

### Statistical Procedure 2

Step 1: Conduct two flow cytometric whole blood platelet assays in healthy human volunteers, specifically, fibrinogen binding (F) and P-selectin expression (P) platelet responses to a PAR4 agonist. Record the two responses for each subject as a percentage of positive platelets (0 to 100%).

### 2.1 Statistical Refinement

Step 2: Apply a logistic transformation to each of the two sets of percentages in Step 1, with the goal of generating two sets of approximately normally distributed sample data, ≈N(y̅_{F}, s_{F}²) and ≈N(y̅_{P}, s_{P}²), or sample data that are at least less skewed and possibly symmetric.

Step 3: Through multiple linear regression, adjust each of the two sets of logistic transformed responses in Step 2 for experimental and other variables that may have influenced the responses but are not believed to be intrinsically important biologically in determining each individual's response (e.g., sub-study, number of days since study initiation, time of day each sample was agonized). Do not adjust for variables which may be intrinsically biologically related (e.g., gender).

Step 4: Standardize (internally studentize) each of the two sets of residuals in Step 3. Move forward to the selection process the two sets of standardized (internally studentized) residuals from the two multiple linear regression analyses.

For this procedure, as outlined in Steps 1 to 4, at this point each subject started with two PAR4 agonist responses (0 to 100%) which were statistically refined to standardized (internally studentized) residuals (−infinity to +infinity) and should contain mostly important, but unknown, intrinsic biological information directly related to either hyper- or hypo-response.

### 2.2 Selection Process

Step 5a (high responders): To select high responders, rank the subjects decreasingly (largest-to-smallest) by the minimum (lesser) of their two refined PAR4 fibrinogen binding and P-selectin expression values (i.e., standardized residuals). Select the upper XX percentile of this ranking, where XX is percentile chosen depending on the variation in the subject population and the stratification desired. In some embodiments, the upper percentile comprises subjects that rank above 65%, 70%, 75%, 80%, 85%, 90%, 95%, 96%, 97%, 98% or 99% of the remaining subjects in the study.

Step 5b (low responders): To select low responders, rank the subjects increasingly (smallest-to-largest) by the maximum (greater) of their two refined PAR4 fibrinogen binding and P-selectin expression values (i.e., standardized residuals). Select the lower XX percentile of this ranking, where XX is percentile chosen depending on the variation in the subject population and the stratification desired. In some embodiments, the lower percentile comprises subjects that rank below 40%, 35%, 30%, 25%, 20%, 15% 10%, 5%, 4%, 3%, 2% or 1% of the remaining subjects in the study.

Step 5c (normal responders): To select the normal responders, rank the subjects increasingly (smallest-to-largest) by the sum of their two absolute deviations from the mean of their two refined PAR4 fibrinogen binding and P-selectin expression values (i.e., standardized residuals). Normal responders will usually be classified as those individuals who fall in a higher percentile than the percentile for the low responders and a lower percentile than the percentile for the high responders.

In certain embodiments of the invention, subjects are considered candidates for PAR4 antagonist therapy based on their categorization as PAR4 agonist responders. For example, subjects who are high responders to PAR4 agonist can be considered optimal candidates for treatment with PAR4 antagonist and subjects who are high responders to PAR4 agonist can be recommended for treatment with PAR4 antagonist. Subjects who are normal responders to PAR4 agonist can be considered candidates for treatment with PAR4 antagonist and subjects who are normal responders to PAR4 agonist can be recommended for treatment with PAR4 antagonist. Subjects who are low responders to PAR4 agonist could be considered candidates for treatment with PAR4 antagonist and subjects who are low responders to PAR4 agonist might be recommended for treatment with PAR4 antagonist to see if positive clinical outcomes are achieved. Subjects who are non-responders—subjects whose platelets do not show platelet activation upon treatment with PAR4 agonist—will typically not be considered candidates for PAR4 antagonist treatment. In certain embodiments, the categorization of subjects as high, normal, low or non-responders to PAR4 agonist allows medical personnel to make a decision on whether a subject is suitable for successful treatment with PAR4 antagonist.

Certain embodiments of the invention provide a method for treating a subject with a PAR4 antagonist, the method comprising: (i) obtaining a blood sample from the subject (ii) contacting a PAR4 agonist with platelets in the sample obtained from the subject, (iii) measuring activation of the platelets and (iv) determining if the subject is a high responder, normal responder, low responder or non-responder to PAR4 agonist based on the amount of platelet activation observed, and (v) administering PAR4 antagonist to the subject if the subject is a high responder, normal responder or low responder to PAR4 agonist.

Certain embodiments of the invention provide a method for treating a subject with PAR4 antagonist, the method comprising: (i) obtaining a blood sample from the subject (ii) contacting a PAR4 agonist with platelets in the sample obtained from the subject, (iii) measuring activation of the platelets and (iv) determining if the subject is a high responder, normal responder, low responder or non-responder to PAR4 agonist based on the amount of platelet activation observed, (v) providing a report to a healthcare provider recommending that the healthcare provider treat the subject with PAR4 antagonist if the subject is a high responder, a normal responder or a low responder.

In some embodiments of the invention, the categorization of subjects can also include a PAR4 antagonist inhibitor test. In some embodiments, measurement of a subject's response to PAR4 antagonist comprises (i) obtaining a blood sample from a subject that has not been treated with a PAR4 antagonist, (ii) pre-incubating a fraction of the sample with a PAR4 antagonist while the remaining fraction is not pre-incubated with the PAR4 antagonist, (iv) treating platelets from the first fraction and the second fraction of the blood sample with a PAR4 agonist in vitro, (v) measuring platelet activation of both the first and second fractions and (vi) determining the percentage inhibition of platelet activation by the PAR4 antagonist by comparing the platelet activation in the first fraction with the platelet activation in the second fraction. In some embodiments, the percentage of platelet inhibition is calculated using the following formula:

((% positive platelets with agonist−% positive platelets with agonist and inhibitor)/(% positive platelets with agonist))×100

In some embodiments of the present invention, subjects can be tested in vitro for percentage of PAR4 antagonist inhibition at a variety of PAR4 antagonist concentrations in order to determine the concentration of antagonist that causes 50% inhibition of platelet activation, a value typically referred to in the art as the IC₅₀ value for the inhibitor. Methods of determining the IC₅₀ value for an inhibitor are well known in the art. Other methods for determining the kinetic values of PAR4 antagonist can also be performed in vitro, as are well known in the art.

In certain embodiments, the invention provides a method for treating a subject with PAR4 antagonist, the method comprising: (i) obtaining a blood sample from a subject that has not been treated with a PAR4 antagonist, (ii) pre-incubating a fraction of the sample with a PAR4 antagonist while the remaining fraction is not pre-incubated with the PAR4 antagonist, (iv) treating platelets from the first fraction and the second fraction of the blood sample with a PAR4 agonist in vitro, (v) measuring platelet activation of both the first and second fractions and (vi) determining the percentage inhibition of platelet activation by the PAR4 antagonist by comparing the platelet activation in the first fraction with the platelet activation in the second fraction, and (vi) treating the subject with a PAR4 antagonist if the second fraction has little to no platelet activation compared to the first fraction. In some embodiments, the subject is treated with PAR4 antagonist if the second fraction less than 25%, 20%, 15%, 10%, 5%, 2% or 1% of the platelet activation of the first fraction.

In certain embodiments, additional information provided by in vitro PAR4 antagonist inhibitor testing can be used to further categorize a subject as discussed in the PAR4 agonist response testing above. For example, if a subject categorized as a high responder to PAR4 agonist shows a higher IC₅₀ value for inhibition with PAR4 antagonist, this can suggest that the subject will require a higher dose of PAR4 antagonist for treatment. By contrast, if a subject categorized as a low responder to PAR4 agonist shows a lower IC₅₀ value for inhibition with PAR4 antagonist, this can suggest that the subject will require a lower dose of PAR4 antagonist for treatment.

In one embodiment, the invention includes a method of identifying a PAR4 agonist responder comprising contacting a PAR4 agonist with platelets in a sample obtained from a subject in need of thereof, wherein the PAR4 agonist activates the platelets. In another embodiment, a method of identifying a PAR4 agonist responder comprises (i) contacting a PAR4 agonist with platelets in a sample obtained from a subject in need thereof, wherein the PAR4 agonist activates the platelets and (ii) measuring platelet activation after the platelets have been in contact with the PAR4 agonist. In other embodiments, a method of identifying a PAR4 agonist responder comprises (i) contacting a PAR4 agonist with platelets in a sample obtained from a subject in need thereof, wherein the PAR4 agonist activates the platelets, (ii) measuring platelet activation after the platelets have been in contact with the PAR4 agonist, and (iii) providing to a healthcare provider the result of measuring the platelet activation. In some embodiments, the PAR4 agonist used in the method of identifying a PAR4 agonist responder activates the platelets equal to or above a high diagnostic score. In some embodiments, the PAR4 agonist activates the platelets to a level which lies in the upper quartile of the distribution observed in a robust sample drawn from the relevant population. In certain embodiments, a PAR4 agonist responder activates the platelets below a normal diagnostic score. In some embodiments, the PAR4 agonist activates the platelets to a level which lies in the lower quartile of the distribution observed in a robust sample drawn from the relevant population. In other embodiments, a PAR4 agonist responder activates the platelets equal to or above a normal diagnostic score and below a high diagnostic score. In some embodiments, the PAR4 agonist activates the platelets to a level which lies in the second quartile and third quartile of the distribution observed in a robust sample drawn from the relevant population.

In certain embodiments, a subject identified as a normal responder to the PAR4 agonist is identified as a normal responder for PAR4 antagonist therapy. In other embodiments, a subject identified as a high responder to the PAR4 agonist is identified as a low responder for PAR4 antagonist therapy. In some embodiments, the method further comprises reporting the results to a healthcare provider and recommending that the healthcare provider administer an increased dose of a PAR4 antagonist to the high PAR4 agonist responder relative to a standard effective dose. In other embodiments, the PAR4 agonist responder identified by the method is a low responder to the PAR4 agonist and is a high responder for a PAR4 antagonist. In some embodiments, the method further comprises recommending that a healthcare provider administer a lower than standard dose of a PAR4 antagonist to the low responder to the PAR4 agonist or recommending that a healthcare provider administer a modified (e.g., lower) amount of a PAR4 antagonist to a subject identified, e.g., as a low responder to the PAR4 agonist.

In some embodiments, the invention includes a method of evaluating platelet activation by a PAR4 agonist in a subject in need thereof, comprising contacting a PAR4 agonist with platelets in a sample obtained from a subject in need thereof, wherein the PAR4 agonist activates the platelets. Other embodiments include a method of evaluating platelet activation by a PAR4 agonist comprising (i) contacting a PAR4 agonist with platelets in a sample obtained from a subject in need thereof, wherein the PAR4 agonist activates the platelets and (ii) measuring platelet activation after the platelets have been in contact with the PAR4 agonist. Other embodiments include a method for evaluating platelet activation by a PAR4 agonist comprising (i) contacting a PAR4 agonist with platelets in a sample obtained from a subject in need thereof, wherein the PAR4 agonist activates the platelets, (ii) measuring platelet activation after the platelets have been in contact with the PAR4 agonist, and (iii) providing to a healthcare provider the result of measuring the platelet activation. In some embodiments, the PAR4 agonist used in a method of evaluating platelet activation as described herein activates the platelets. In certain embodiments, the PAR4 agonist used in a method as described herein activates the platelets above a high diagnostic score. In certain embodiments, the PAR4 agonist used in a method as described herein activates the platelets at a level lower than a normal diagnostic score. In certain embodiments, the PAR4 agonist used in a method as described herein activates the platelets equal to or above a normal diagnostic score and below a high diagnostic score.

In other aspects, the invention includes a method for identifying a PAR4 antagonist therapy responder comprising contacting a PAR4 agonist with platelets in a sample obtained from a subject in need of PAR4 antagonist therapy, wherein the PAR4 agonist activates the platelets. In some embodiments, the method for identifying a PAR4 antagonist therapy responder comprises (i) contacting a PAR4 agonist with platelets in a sample obtained from a subject in need of PAR4 antagonist therapy, wherein the PAR4 agonist activates the platelets and (ii) measuring platelet activation after the platelets have been in contact with the PAR4 agonist. In certain embodiments, a method for identifying a PAR4 antagonist therapy responder comprises (i) contacting a PAR4 agonist with platelets in a sample obtained from a subject in need in need of PAR4 antagonist therapy, wherein the PAR4 agonist activates the platelets, (ii) measuring platelet activation after the platelets have been in contact with the PAR4 agonist, and (iii) providing to a healthcare provider the result of measuring the platelet activation. In some embodiments, the PAR4 agonist used in the method of identifying a PAR4 antagonist therapy responder activates the platelets above a normal diagnostic score. In certain cases, the subject exhibiting platelet activation by a PAR4 agonist above a normal diagnostic score is a low responder for PAR4 antagonist therapy. In certain embodiments, the PAR4 agonist used in the method of identifying a PAR4 antagonist therapy responder activates the platelets equal to or above a normal diagnostic score and below a high diagnostic score; the subject is a normal responder for PAR4 antagonist therapy. In some cases, the subject exhibiting platelet activation by a PAR4 agonist above a high diagnostic score is a high responder for PAR4 antagonist therapy. In some embodiments, the method further comprises providing the results to a healthcare provider and/or recommending a dosing regimen to a healthcare provider for administering a PAR4 antagonist to the subject based on the results of an assay described herein. In certain embodiments, the invention includes a method of identifying a non-responder to a PAR4 agonist comprising contacting a PAR4 agonist with platelets in a sample obtained from the subject in need in need thereof, wherein the PAR4 agonist does not activate the platelets. In other embodiments, the invention includes a method of identifying a non-responder to a PAR4 agonist comprising contacting a PAR4 agonist with platelets in a sample obtained from the subject in need in need thereof and measuring platelet activation, wherein the PAR4 agonist does not activate the platelets. Non-responders to a PAR4 agonist have no platelet activation when the platelets come in contact with the PAR4 agonist. The non-responsiveness can be measured by comparing platelet activation by a PAR4 agonist with platelet activation by a negative control, e.g., PAR1 agonist.

In other embodiments, the invention provides a method of evaluating responsiveness of a subject to PAR4 antagonist therapy comprising contacting a PAR4 agonist with platelets in a sample obtained from the subject in need in need of PAR4 antagonist therapy, wherein the PAR4 agonist activates the platelets. In certain embodiments, a method of evaluating responsiveness of a subject to PAR4 antagonist therapy comprises (i) contacting a PAR4 agonist with platelets in a sample obtained from a subject in need of PAR4 antagonist therapy, wherein the PAR4 agonist activates the platelets and (ii) measuring platelet activation after the platelets have been in contact with the PAR4 agonist. In some embodiments, a method of evaluating responsiveness of a subject to PAR4 antagonist therapy comprises (i) contacting a PAR4 agonist with platelets in a sample obtained from a subject in need in need of PAR4 antagonist therapy, wherein the PAR4 agonist activates the platelets, (ii) measuring platelet activation after the platelets have been in contact with the PAR4 agonist, and (iii) providing the results to a healthcare provider and/or recommending a dosing regimen to a healthcare provider for administering a PAR4 antagonist to the subject based on the results of an assay described herein. In some embodiments, the PAR4 agonist used in the method of evaluating platelet activation by a PAR4 agonist activates the platelets above a high diagnostic score. In certain embodiments, the PAR4 agonist used in the method activates the platelets below the normal diagnostic score. In other embodiments, the PAR4 agonist used in the method activates the platelets equal to or above a normal diagnostic score and below a high diagnostic score.

In some embodiments, the invention includes a method of identifying a PAR4 agonist responder comprising contacting a PAR4 agonist with platelets in a sample obtained from a subject in need thereof, wherein the PAR4 agonist activates the platelets below a normal diagnostic score, equal to or above a normal diagnostic score and below a high diagnostic score, or above high diagnostic score. In other embodiments, the method for identifying a PAR4 agonist responder comprises (i) contacting a PAR4 agonist with platelets in a sample obtained from a subject in need thereof, and (ii) measuring platelet activation, wherein the PAR4 agonist activates the platelets below a normal diagnostic score, equal to or above a normal diagnostic score and below a high diagnostic score, or above high diagnostic score after being in contact with the platelets. In yet other embodiments, the method for identifying a PAR4 agonist responder comprises (i) contacting a PAR4 agonist with platelets in a sample obtained from a subject in need thereof, (ii) measuring platelet activation, and (iii) providing to a healthcare provider the result of measuring the platelet activation, wherein the PAR4 agonist activates the platelets below a normal diagnostic score, above a normal diagnostic score and below a high diagnostic score, or above high diagnostic score after being in contact with the platelets.

In certain embodiment, the invention includes a method of identifying a non-responder to a PAR4 antagonist comprising contacting a PAR4 agonist with platelets in a sample obtained from the subject in need in need thereof, wherein the PAR4 agonist does not activate the platelets. In other embodiments, the invention includes a method of identifying a non-responder to a PAR4 agonist comprising contacting a PAR4 agonist with platelets in a sample obtained from the subject in need in need thereof and measuring platelet activation, wherein the PAR4 agonist does not activate the platelets. In other embodiments, the method for identifying a non-responder for a PAR4 agonist comprises (i) contacting a PAR4 agonist with platelets in a sample obtained from a subject in need thereof, (ii) measuring platelet activation, and (iii) providing to a healthcare provider the result of measuring the platelet activation, wherein the PAR4 agonist does not activates the platelets after being in contact with the platelets.

In other embodiments, the invention is directed to a method for identifying a PAR4 antagonist therapy responder comprising contacting a PAR4 agonist with platelets in a sample obtained from a subject in need of PAR4 antagonist therapy, wherein the PAR4 agonist activates the platelets and wherein platelet activation by the PAR4 agonist indicates that the subject is a PAR4 antagonist therapy responder. In yet other embodiments, the PAR4 agonist activates the platelets below a normal diagnostic score, equal to or above a normal diagnostic score and below a high diagnostic score, or above high diagnostic score after being in contact with the platelets. In still other embodiments, the invention is directed to a method for identifying a PAR4 antagonist therapy responder comprising (i) contacting a PAR4 agonist with platelets in a sample obtained from a subject in need of PAR4 antagonist therapy, and (ii) measuring platelet activation after the platelets have been in contact with the PAR4 agonist, wherein the PAR4 agonist activates the platelets below a normal diagnostic score, equal to or above a normal diagnostic score and below a high diagnostic score, or above high diagnostic score after being in contact with the platelets, and wherein platelet activation indicates that the subject is a PAR4 antagonist therapy responder. In yet other embodiments, a method for identifying a PAR4 antagonist therapy responder comprises (i) contacting a PAR4 agonist with platelets in a sample obtained from a subject in need of PAR4 antagonist therapy, (ii) measuring platelet activation after the platelets have been in contact with the PAR4 agonist, wherein the PAR4 agonist activates the platelets below a normal diagnostic score, equal to or above a normal diagnostic score and below a high diagnostic score, or above high diagnostic score after being in contact with the platelets, wherein the platelet activation indicates that the subject is a PAR4 antagonist therapy responder, and (iii) providing the results to a healthcare provider and/or recommending a dosing regimen to a healthcare provider for administering a PAR4 antagonist to the subject based on the results of an assay described herein.

In some embodiments, the invention includes a method for evaluating platelet activation by a PAR4 agonist in a subject in need thereof comprising contacting a PAR4 agonist with platelets in a sample obtained from the subject, wherein the PAR4 agonist activates the platelets below a normal diagnostic score, equal to or above a normal diagnostic score and below a high diagnostic score, or above a high diagnostic score. In some embodiments, the invention includes a method for evaluating platelet activation by a PAR4 agonist in a subject in need thereof comprising (i) contacting a PAR4 agonist with platelets in a sample obtained from the subject, wherein the PAR4 agonist activates the platelets and (ii) measuring activation of the platelets below a normal diagnostic score, equal to or above a normal diagnostic score and below a high diagnostic score, or above a high diagnostic score after being in contact with the platelets.

In some embodiments, the invention is directed to a method for evaluating responsiveness of a subject to PAR4 antagonist therapy comprising contacting a PAR4 agonist with platelets in a sample obtained from the subject in need of the therapy, wherein the PAR4 agonist activates the platelets below a normal diagnostic score after being in contact with the PAR4 agonist. In other embodiments, the platelet activation equal to or above a normal diagnostic score indicates that the subject is a high responder for PAR4 antagonist therapy. In certain embodiments, the invention is directed to a method for evaluating responsiveness of a subject to PAR4 antagonist therapy comprising (i) contacting a PAR4 agonist with platelets in a sample obtained from the subject in need of the PAR4 antagonist therapy, wherein the PAR4 agonist activates the platelets below a normal diagnostic score and (ii) measuring the platelet activation, wherein platelet activation below a normal diagnostic score indicates that the subject is a high responder for PAR4 antagonist.

In some embodiments, the invention is directed to a method for evaluating responsiveness of a subject to PAR4 antagonist therapy comprising contacting a PAR4 agonist with platelets in a sample obtained from the subject in need of the therapy, wherein the PAR4 agonist activates the platelets above a high diagnostic score after being in contact with the PAR4 agonist. In other embodiments, the platelet activation above a high diagnostic score indicates that the subject is a low responder for PAR4 antagonist therapy. In certain embodiments, the invention is directed to a method for evaluating responsiveness of a subject to PAR4 antagonist therapy comprising (i) contacting a PAR4 agonist with platelets in a sample obtained from the subject in need of the PAR4 antagonist therapy, wherein the PAR4 agonist activates the platelets below a normal diagnostic score and (ii) measuring the platelet activation, wherein platelet activation below a normal diagnostic score indicates that the subject is a high responder for PAR4 antagonist.

The result of measuring platelet activation can show that the subject is a normal responder, a low responder (hypo responder), or a high responder (hyper responder) to a PAR4 agonist. In one embodiment, the subject who is a normal responder for a PAR4 agonist is a normal responder for PAR4 antagonist therapy. In another embodiment, the subject who is a low responder for a PAR4 agonist is a high responder for a PAR4 antagonist. In other embodiments, the subject who is a high responder for a PAR4 agonist is a low responder for a PAR4 antagonist.

The present invention also includes a method of identifying a PAR4 agonist responder comprising (i) contacting a PAR4 agonist with platelets in a sample obtained from a subject in need of PAR4 antagonist therapy, wherein the PAR4 agonist activates the platelets and (ii) administering a PAR4 antagonist. In another embodiment, a method of identifying a PAR4 agonist responder comprises (i) contacting a PAR4 agonist with platelets in a sample obtained from a subject thought to be in need of PAR4 antagonist therapy, wherein the PAR4 agonist activates the platelets, (ii) measuring platelet activation after the platelets have been in contact with the PAR4 agonist, and (iii) administering a PAR4 antagonist. In other embodiments, a method of identifying a PAR4 agonist responder comprises (i) contacting a PAR4 agonist with platelets in a sample obtained from a subject in need of PAR4 antagonist therapy, wherein the PAR4 agonist activates the platelets, (ii) measuring platelet activation after the platelets have been in contact with the PAR4 agonist, (iii) providing to a healthcare provider the result of measuring the platelet activation, and (iv) administering a PAR4 antagonist. In some embodiments, the PAR4 agonist used in the method of identifying a PAR4 agonist responder activates the platelets below a normal diagnostic score. In other embodiments, a standard dose of the PAR4 antagonist is administered to the subject when the subject demonstrates platelet activation by a PAR4 agonist equal to or above a normal diagnostic score but below a high diagnostic score. In certain embodiments, the PAR4 agonist used in the method activates the platelets equal to or above a high diagnostic score, i.e., high PAR4 agonist responder. A high PAR4 agonist responder can receive a higher than standard dose of the PAR4 antagonist.

The present invention also includes a method for reducing or decreasing platelet aggregation in a subject thought to be in need of PAR4 antagonist therapy, comprising (i) submitting a platelet sample obtained from the subject for platelet activation testing with a PAR4 agonist, wherein the PAR4 agonist activates the platelets, and (ii) administering an effective amount of a PAR4 antagonist to the subject, wherein the effective amount is determined based on the platelet activation testing. The method for reducing or decreasing platelet aggregation can result in treatment, prevention, or amelioration of a disease or condition associated with thromboembolism.

The present invention also includes a method for inhibiting or preventing platelet aggregation in a subject thought to be in need of PAR4 antagonist therapy, comprising (i) submitting a platelet sample obtained from the subject for platelet activation testing with a PAR4 agonist, wherein the PAR4 agonist activates the platelets below a normal diagnostic score, equal to or above a normal diagnostic score and below a high diagnostic score, or equal to or above a high diagnostic score, and (ii) administering an effective amount of a PAR4 antagonist to the subject, wherein the effective amount is determined based on the platelet activation testing. The method for reducing or decreasing platelet aggregation can result in treatment, prevention, or amelioration of a disease or condition associated with thromboembolism.

In some embodiments, the invention provides a method for treating, preventing, or ameliorating a disease or condition associated with thromboembolism in a subject suspected of having or being susceptible to a disease or condition associated with thromboembolism comprising (i) submitting a platelet sample obtained from the subject for platelet activation testing with a PAR4 agonist, and (ii) administering different doses of a PAR4 antagonist based on the type of PAR4 agonist responder, with a low PAR4 agonist responder receiving less PAR4 antagonist, with a high PAR4 agonist responder receiving more PAR4 antagonist, with a normal responder receiving a standard dose of agonist as shown in Table 1.

In other embodiments, the method of reducing or decreasing platelet aggregation or treating, preventing or ameliorating a disease or condition associated with thromboembolism comprises (i) submitting a platelet sample obtained from a subject suspected of having or being susceptible to a disease or condition associated with thromboembolism for platelet activation testing with a PAR4 agonist, (ii) receiving a result of the platelet activation testing, and (iii) administering an effective amount of a PAR4 antagonist to the subject if the PAR4 agonist activates the platelets, wherein the effective amount of the PAR4 antagonist is determined based on the results of platelet activation testing.

In other embodiments, the method of inhibiting or preventing platelet aggregation or treating, preventing or ameliorating a disease or condition associated with thromboembolism comprises (i) submitting a platelet sample obtained from a subject suspected of having or being susceptible to a disease or condition associated with thromboembolism for platelet activation testing with a PAR4 agonist, (ii) receiving a result of the platelet activation testing, and (iii) administering an effective amount of a PAR4 antagonist to the subject wherein the effective amount of the PAR4 antagonist is determined based on the results of platelet activation testing.

In certain embodiments, the method of reducing or decreasing platelet aggregation or treating, preventing or ameliorating a disease or condition associated with thromboembolism comprises (i) contacting a PAR4 agonist with platelets in a sample obtained from a subject suspected of having or being susceptible to a disease or condition associated with thromboembolism, (ii) measuring platelet activation, and (iii) administering an effective amount of a PAR4 antagonist to the subject wherein the effective amount of the PAR4 antagonist is determined based on the results of platelet activation testing. In some embodiments, the present invention includes a method for the treatment of a thromboembolic disorder or the primary prophylaxis of a thromboembolic disorder, which includes the steps of administering to a patient (for example, a human) in need thereof a therapeutically effective amount of a compound of Formula V, or stereoisomers, tautomers, pharmaceutically acceptable salts, prodrug esters, or solvates thereof, wherein the thromboembolic disorder is selected from arterial cardiovascular thromboembolic disorders, venous cardiovascular thromboembolic disorders, thromboembolic disorders in the chambers of the heart or in the peripheral circulation, arterial cerebrovascular thromboembolic disorders and venous cerebrovascular thromboembolic disorders.

The disease or condition associated with thromboembolism can include, is not limited to, arterial cardiovascular thromboembolic disorders, venous cardiovascular or cerebrovascular thromboembolic disorders, and thromboembolic disorders in the chambers of the heart or in the peripheral circulation. The disease or condition associated with thromboembolism can also include specific disorders selected from, but not limited to, unstable angina or other acute coronary syndromes, atrial fibrillation, first or recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from medical implants, devices, or procedures in which blood is exposed to an artificial surface that promotes thrombosis. The medical implants or devices include, but are not limited to: prosthetic valves, artificial valves, indwelling catheters, stents, blood oxygenators, shunts, vascular access ports, ventricular assist devices and artificial hearts or heart chambers, and vessel grafts. The procedures include, but are not limited to: cardiopulmonary bypass, percutaneous coronary intervention, and hemodialysis. In another embodiment, the disease or condition associated with thromboembolism includes acute coronary syndrome, stroke, deep vein thrombosis, and pulmonary embolism.

The effective dose of the PAR4 antagonist can be a biologically active dose. A biologically active dose is a dose that will inhibit cleavage and/or signaling of PAR4 and have an anti-thrombotic effect. Desirably, the PAR4 antagonist has the ability to reduce the activity of PAR4 by at least 5%, 10%, 20%, 30%, 40%, 50%, 60%, 70%, 80%, 90%, 100%, or more than 100% below untreated control levels. The levels of PAR4 in platelets is measured by any method known in the art, including, for example, receptor binding assay, platelet aggregation, platelet activation assays (e.g., P-selectin expression by flow cytometry), Western blot or ELISA analysis using PAR4 cleavage sensitive antibodies. Alternatively, the biological activity of PAR4 is measured by assessing cellular signaling elicited by PAR4 (e.g., calcium mobilization or other second messenger assays).

In some embodiments, a therapeutically effective amount of a PAR4 compound is from about less than 100 mg/kg, 50 mg/kg, 10 mg/kg, 5 mg/kg, 1 mg/kg, or less than 1 mg/kg. In certain embodiments, the therapeutically effective amount of the PAR4 compound is less than 5 mg/kg. In other embodiments, the therapeutically effective amount of the PAR4 compound is less than 1 mg/kg. Effective doses vary, as recognized by those skilled in the art, depending on route of administration, post administration modification of drug effectiveness and excipient usage.

According to the present invention, subjects whose samples containing platelets come into a contact with a PAR4 agonist can be divided into three categories, a low responder to the PAR4 agonist, a normal responder to the PAR4 agonist, and a high responder to the PAR4 agonist. Determination of the subject into each category can be based on the diagnostic score of each subject, which can be generated after measuring platelet activation by a PAR4 agonist in a robust population sample of subjects. For instance, platelet activation can be measured by measuring changes of the platelets at different levels, e.g., by measuring changes in the platelet cytoplasm, by measuring changes of the platelet membrane, by measuring changes in the levels of analytes released by platelets, by measuring the changes in the morphology of the platelet, by measuring the ability of platelets to form thrombi or platelet aggregates in flowing or stirred whole blood, to measure the ability of platelets to adhere to a static surface which is derivatised with relevant ligands (e.g., Von Willebrand Factor, Collagen, fibrinogen, or other extracellular matrix proteins or synthetic fragments of any of the proteins), or by measuring the changes in the shape of the platelets, or any combinations thereof

In one embodiment, platelet activation is measured by changes in the levels of one or more analytes released by platelets. The one or more analytes released by platelets include, but are not limited to, P-selectin (CD62p), CD63, ATP, or any combination thereof. For example, the level of analytes secreted by platelets can be measured by one or more antibodies specifically binding to the analytes. Non-limiting examples of the antibody specifically binding to the analytes, e.g., P-Selectin, is disclosed elsewhere herein.

In another embodiment, platelet activation is measured by the ability of platelets to adhere to a static surface which is derivatized with one or more relevant ligands (e.g., von Willebrand factor, collagen, other extracellular matrix proteins, synthetic fragment thereof, or any combination thereof). For example, platelet activation can be detected by measuring the proteins binding to activated GPIIb/IIIa or activated platelet membrane. Non-limiting examples of the antibodies and proteins binding to GPIIb/IIIa include PAC-1 or fibrinogen. In some embodiments, platelet activation is measured by detecting annexin V, which binds to the activated membrane.

In other embodiments, platelet activation is measured by the degree of phosphorylation of vasodilator-stimulated phosphoprotein (VASP) upon platelet activation. In some embodiments, platelet activation is measured by the level of platelet-leukocyte aggregates.

In certain embodiments, platelet activation is measured by a change in the platelets' shape. Resting platelets or thrombocytes are small, disk shaped clear cell fragments (i.e., cells that do not have a nucleus), 2-3 μm in diameter, which are derived from fragmentation of precursor megakaryocytes. Activated platelets change in shape to become more spread, and pseudopods form on their surface. Thus they assume a stellate shape. The changes of the platelets' shape can be measured by microscopy, flow cytometry, or platelet aggregometry.

In another embodiment, platelet activation is measured by a change in the level of expression of a platelet activation marker on the membrane of the cell. The outer most layer of the platelet is a surface coat made up of glycoproteins, which include various receptors binding to adhesive agents, aggregating agents, extracellular matrix proteins, and procoagulant factors. The receptors expressed on platelets include, but are not limited to, the heterodimeric integrins alpha-beta complexes (e.g., GPIIb/IIIa (alphaIIb/beta3), GPIa/IIa (beta 1/alpha2), beta1/alpha6, GP VI, GPIb-alpha, GP1b-beta, GPIX, or GP V), receptors for the CH domain of immunoglobulin and other receptors for, e.g., adenosine diphosphate (ADP), collagen, epinephrine, thrombin, etc. In other embodiments, platelet activation markers include one or more proteins where the expression level changes significantly between resting and activated platelets. The level of a marker can go up or down and significant differed can be observed between levels observed on resting and activated platelets. In still other embodiments, platelet marker activation status is a change in the level of one or more proteins expressed on the activated platelets compared to resting platelets.

In one embodiment, the platelet activation marker is the binding of fibrinogen. Fibrinogen, the principal protein of vertebrate blood clotting, is a hexamer, containing two sets of three different chains (α, β, and γ), linked to each other by disulfide bonds. The N-terminal sections of these three chains contain the cysteines that participate in the cross-linking of the chains. The C-terminal parts of the α, β and γ chains contain a domain of about 225 amino-acid residues, which can function as a molecular recognition unit and is implicated in protein-protein interactions. The a chain of human fibrinogen is known as No. P02679 at UniProtKB/Swiss-Prot (SEQ ID NO: 35) and is shown at Table 2. Amino acids 1-19 of the chain are the signal peptide, and amino acids 623 to 864 are the C-terminal domain. The β chain of human fibrinogen is known as No. P02675 at UniProtKB/Swiss-Prot (SEQ ID NO: 36) and is shown at Table 2. Amino acids 1 to 30 of the chain are the signal peptide, and amino acids 232 to 488 are the C-terminal domain. The γ chain of human fibrinogen is known as No. P02679 at UniProtKB/Swiss-Prot (SEQ ID NO: 37) and is shown at Table 2. The amino acids 1 to 26 are its signal peptide, and amino acids 170 to 416 (247aa) are the C-terminal domain.

TABLE 2
Fibrinogen Sequence

| Chains | Amino acid sequence |
|---|---|
| α | 10 20 30 40 50 60 |
| (SEQ ID | MFSMRIVCLV LSVVGTAWTA DSGEGDFLAE GGGVRGPRVV ERHQSACKDS DWPFCSDEDW |
| NO: 35) | 70 80 90 100 110 120 |
| | NYKCPSGCRM KGLIDEVNQD FTNRINKLKN SLFEYQKNNK DSHSLTTNIM EILRGDFSSA |
| | 130 140 150 160 170 180 |
| | NNRDNTYNRV SEDLRSRIEV LKRKVIEKVQ HIQLLQKNVR AQLVDMKRLE VDIDIKIRSC |
| | 190 200 210 220 230 240 |
| | RGSCSRALAR EVDLKDYEDQ QKQLEQVIAK DLLPSRDRQH LPLIKMKPVP DLVPGNFKSQ |
| | 250 260 270 280 290 300 |
| | LQKVPPEWKA LTDMPQMRME LERPGGNEIT RGGSTSYGTG SETESPRNPS SAGSWNSGSS |
| | 310 320 330 340 350 360 |
| | GPGSTGNRNP GSSGTGGTAT WKPGSSGPGS TGSWNSGSSG TGSTGNQNPG SPRPGSTGTW |
| | 370 380 390 400 410 420 |
| | NPGSSERGSA GHWTSESSVS GSTGQWHSES GSFRPDSPGS GNARPNNPDW GTFEEVSGNV |
| | 430 440 450 460 470 480 |
| | SPGTRREYHT EKLVTSKGDK ELRTGKEKVT SGSTTTTRRS CSKTVTKTVI GPDGHKEVTK |
| | 490 500 510 520 530 540 |
| | EVVTSEDGSD CPEAMDLGTL SGIGTLDGFR HRHPDEAAFF DTASTGKTFP GFFSPMLGEF |
| | 550 560 570 580 590 600 |
| | VSETESRGSE SGIFTNTKES SSHHPGIAEF PSRGKSSSYS KQFTSSTSYN RGDSTFESKS |
| | 610 620 630 640 650 660 |
| | YKMADEAGSE ADHEGTHSTK RGHAKSRPVR DCDDVLQTHP SGTQSGIFNT KLPGSSKIFS |
| | 670 680 690 700 710 720 |
| | VYCDQETSLG GWLLIQQRMD GSLNFNRTWQ DYKRGFGSLN DEGEGEFWLG NDYLHLLTQR |
| | 730 740 750 760 770 780 |
| | GSVLRVELED WAGNEAYAEY HFRVGSEAEG YALQVSSYEG TAGDALIEGS VEEGAEYTSH |
| | 790 800 810 820 830 840 |
| | NNMQFSTFDR DADQWEENCA EVYGGGWWYN NCQAANLNGI YYPGGSYDPR NNSPYEIENG |
| | 850 860 |
| | VVWVSFRGAD YSLRAVRMKI RPLVTQ |
| β | 10 20 30 40 50 60 |
| (SEQ ID | MKRMVSWSFH KLKTMKHLLL LLLCVFLVKS QGVNDNEEGF FSAKGHRPLD KKREEAPSLR |
| NO: 36) | 70 80 90 100 110 120 |
| | PAPPPISGGG YRARPAKAAA TQKKVERKAP DAGGCLHADP DLGVLCPTGE QLQEALLQQE |
| | 130 140 150 160 170 180 |
| | RPIRNSVDEL NNNVEAVSQT SSSSFQYMYL LKDLWQKRQK QVKDNENVVN EYSSELEKHQ |
| | 190 200 210 220 230 240 |
| | LYIDETVNSN IPTNLRVLRS ILENLRSKIQ KLESDVSAQM EYCRTPCTVS CNIPVVSGKE |
| | 250 260 270 280 290 300 |
| | CEEIIRKGGE TSEMYLIQPD SSVKPYRVYC DMNTENGGWT VIQNRODGSV DFGRKWDPYK |
| | 310 320 330 340 350 360 |
| | QGFGNVATNT DGKNYCGLPG EYWLGNDKIS QLTRMGPTEL LIEMEDWKGD KVKAHYGGFT |
| | 370 380 390 400 410 420 |
| | VQNEANKYQI SVNKYRGTAG NALMDGASQL MGENRTMTIH NGMFFSTYDR DNDGWLTSDP |
| | 430 4400 450 460 470 480 |
| | RKQCSKEDGG GWWYNRCHAA NPNGRYYWGG QYTWDMAKHG TDDGVVWMNW KGSWYSMRKM |
| | 440 |
| | SMKIRPFFPQ Q |
| γ | 10 20 30 40 50 60 |
| (SEQ ID | MSWSLHPRNL ILYFYALLFL SSTCVAYVAT RDNCCILDER FGSYCPTTCG IADFLSTYQT |
| NO: 37) | 70 80 90 100 110 120 |
| | KVDKDLQSLE DILHQVENKT SEVKQLIKAI QLTYNPDESS KPNMIDAATL KSRKMLEEIM |
| | 130 140 150 160 170 180 |
| | KYEASILTHD SSIRYLQEIY NSNNQKIVNL KEKVAQLEAQ C4EPCKDTVQ IRDITGKDCQ |
| | 190 200 210 220 230 240 |
| | DIANKGAKQS GLYFIKPLKA NQQFLVYCEI DGSGNGWTVF QKRLDGSVDF KKNWIQYKEG |
| | 250 260 270 280 290 300 |
| | FGHLSPTGTT EFWLGNEKIH LISTQSAIPY ALRVELEDWN GRTSTADYAM FKVGPEADKi |
| | 310 320 330 340 350 360 |
| | RLTYAYFAGG DAGDAFDGFD FGDDPSDKFF TSHNGMQFST WDNDNDKFEG NCAEQDGSGW |
| | 370 380 390 400 410 420 |
| | WMNKCHAGHL NGVYYQGGTY SKASTPNGYD NGIIWATWKT RWYSMKKTTM KIIPFNRLTI |
| | 430 440 450 |
| | GEGQQHHLGG AKQVRPEHPA ETEYDSLYPE DDL |

In another embodiment, the platelet activation marker is P-selectin. P-selectin is also known as CD62 antigen-like family member P, CD62P antigen, granule membrane protein 140 (GMP-140), leukocyte-endothelial cell adhesion molecule 3 (LECAM3), platelet alpha-granule membrane protein (PADGEM), or SELP. P-selectin is encoded by the SELP gene and functions as a cell adhesion molecule (CAM) on the surfaces of activated endothelial cells, which line the inner surface of blood vessels, and activated platelets. At resting state, it is stored in granules called Weibel-Palade bodies in endothelial cells, and a-granules in unactivated platelets. P-selectin is constitutively expressed on megakaryocytes (the precursor of platelets) and endothelial cells. The expression of P-selectin consists of two distinct mechanisms. First, P-selectin is synthesized by megakaryocytes and endothelial cells, where it is sorted into the membranes of secretory granules. When megakaryocytes and endothelial cells are activated by the action of agonists such as thrombin, P-selectin is rapidly translocated to the plasma membrane from granules. Second, the increased level of mRNA and protein of P-selectin is induced by inflammatory mediators such as tumor necrosis factor-a (TNF-a), LPS, or interleukin-4 (IL-4). The human P-Selectin sequence is known as No. P16109 at UniProtKB/Swiss-Prot (SEQ ID NO: 38) and is shown in Table 3.

TABLE 3
P-Selectin Sequence(SEQ ID NO: 38)

```
                10 20 30 40 50 60
MANCQIAILY QRFQRVVFGI SQLLCFSALI SELTNQKEVA AWTYMYSTKA YSWNISRKYC
                70 80 90 100 110 120
QNRYTDLVAI QNKNEIDYLN KVLPYYSSYY WIGIRKNNKT WTWVGTKKAL TNEAENWADN
              130 140 150 160 170 180
EPNNKRNNED CVETYIKSPS APGKWNDEHC LKKKHALCYT ASCQDMSCSK QGECLETIGN
              190 200 210 220 230 240
YTCSCYPGFY GPECEYVREC GELELPQHVL MNCSHPLGNF SFNSQCSFHC TDGYQVNGPS
              250 260 270 280 290 300
KLECLASGIW TNKFPQCLAA QCFPLKIPER GNMTCLHSAK AFQHQSSCSF SCEEGFALVG
              310 320 330 340 350 360
PEVVQCTASG VWTAPAPVCK AVQCQHLEAP SEGTMDCVHP LTAFAYGSSC KFECQPGYRV
              370 380 390 400 410 420
RGLDMLRCID SGHWSAPLPT CEAISCEPLE SPVHGSMDCS PSLRAFQYDT NCSFRCAEGF
              430 440 450 460 470 480
MLRGADIVRC DNLGQWTAPA PVCQALQCQD LPVPNEARVN CSHPFGAFRY QSVCSFTCNE
              490 500 510 520 530 540
GLLLVGASVL QCLATGNWNS VPPECQAIPC TPLLSPQNGT MTCVQPLGSS SYKSTCQFIC
              550 560 570 580 590 600
DEGYSLSGPE RLDCTRSGRW TDSPPMCEAI KCPELFAPEQ GSLDCSDTRG EFNVGSTCHF
              610 620 630 640 650 660
SCDNGFKLEG PNNVECTTSG RWSATFPTCK GIASLPTPGL QCPALTTPGQ GTMYCRHHPG
              670 680 690 700 710 720
TFGFNTTCYF GCNAGFTLIG DSTLSCRPSG QWTAVTPACR AVKCSELHVN KPIAMNCSNL
              730 740 750 760 770 780
WGNFSYGSIC SFHCLEGQLL NGSAQTACQE NGHWSTTVPT CQAGPLTIQE ALTYFGGAVA
              790 800 810 820 830
STIGLIMGGT LLALLRKRFR QKDDGKCPLN PHSHLGTYGV FTNAAFDPSP
```

In certain embodiments the platelet activation is detected by reacting an antibody specific for the phosphorylation of tyrosines in certain cytoplasmic platelet proteins after a platelet sample has been engaged with a PAR4 agonists. The level of tyrosine phosphorylation of individual proteins can be measured by e.g., immunoblot or mass spectrometry.

In certain embodiments the platelet activation is detected by measuring the level of protein fragments in the platelet releasate after contacting a platelet sample with a PAR4 agonists. The level of protein fragments can be measured by e.g., mass spectrometry.

In certain embodiments, the platelet activation marker is detected by an antibody or antigen-binding fragment thereof that specifically binds to the marker. In certain embodiments, an antibody or antigen-binding fragment thereof is polyclonal or monoclonal.

In some embodiments, the antibody that specifically binds to fibrinogen is an anti-fibrinogen antibody. Anti-fibrinogen antibody includes, but is not limited to, F0111, LS-B2573, LS-B697, LS-B5249, LS-B381, LS-B3048, LS-B7075, LS-052057, LS-C109177, LS-C150799, MCA2760, 4440-8004, NBP1-33582, NB600-926, NBP1-47442, NBP2-11515, NB120-10070, NBP1-96183, NBP1-96180, or any combinations thereof. F0111 is available from Dako and is a polyclonal rabbit anti-human fibrinogen antibody conjugated to fluoresceine isothiocyanate isomer (FITC). The titre of the antibody is 100 mg/L (i.e., mg of antigen to be added to 1 L of antibody to reach equivalence point). F/P ratio of F0111 is E_{495 nm}/E_{278 nm}=0.65 0.05 corresponding to a molar FITC/protein ratio of 2.5. The antibody reacts with native fibrinogen as well as with the fibrinogen fragments D and E. LS-B2573 is available from LifeSpan BioSciences, Inc. and is a polyclonal sheep anti-human fibrinogen antibody. LS-B697 and LS-B381 are also available from LifeSpan BioSciences, Inc. and are a polyclonal goat anti-human fibrinogen antibody. LS-B5249, LS-C109177, and LS-C150799 are also available from LifeSpan BioSciences, Inc. and are a polyclonal Rabbit anti-Human fibrinogen Antibody. LS-B3048, LS-B7075, and LS-052057 are available from LifeSpan BioSciences, Inc. and are a monoclonal mouse anti-human fibrinogen antibody. MCA2760 is available from Bio-Rad Laboratories and is a monoclonal mouse anti-human fibrinogen antibody. 4440-8004 is available from Bio-Rad Laboratories and is a polyclonal sheep anti-human fibrinogen antibody. It is available as a conjugate with either FITC or horse radish peroxidase. NBP1-33582 is available from Novus Biologicals and is a polyclonal rabbit anti-human fibrinogen antibody. NB600-926 is available from Novus Biologicals and is a polyclonal goat anti-human fibrinogen antibody. NBP1-47442 is available from Novus Biologicals and is a polyclonal mouse anti-human fibrinogen antibody. NBP2-11515 is available from Novus Biologicals and is a polyclonal mouse anti-human fibrinogen antibody. NB120-10070 is available from Novus Biologicals and is a monoclonal mouse anti-human fibrinogen antibody. NBP1-96183 and NBP1-96180 are available from Novus Biologicals and are a monoclonal rat anti-human fibrinogen antibody.

In other embodiments, the antibody against fibrinogen binds to native fibrinogen and the fibrinogen fragments D and E. In yet other embodiments, the antibody against fibrinogen is a rabbit polyclonal against human fibrinogen available from commercial sources. In a specific embodiment, the antibody against fibrinogen is a purified immunoglobulin fraction of rabbit antiserum conjugated with fluorescein isothiocyanate (FTIC) isomer (F0111) (available from Dako).

In certain embodiments, a normal diagnostic score comprises a percentage of activated platelets, which is about 20%, about 25%, about 30%, about 35%, about 40%, about 45%, or about 50% platelets being activated, measured by an anti-fibrinogen antibody, e.g., purified immunoglobulin fraction of rabbit antiserum conjugated with fluorescein isothiocyanate (FTIC) isomer, e.g., F0111. In other embodiments, a normal diagnostic score comprises the percentile selected from the horizontal line of FIG. 1 which represents the normal diagnostic score (e.g., 25%) within a distribution curve of platelet activation measurements observed in a population sample. In some embodiments, a high diagnostic score comprises a percentage of activated platelets, which is about 40%, about 45%, about 50%, about 55%, about 60%, about 65%, about 70%, about 75%, about 80%, about 85%, about 90%, about 95% platelets being activated, measured by an anti-fibrinogen antibody, e.g., purified immunoglobulin fraction of rabbit antiserum conjugated with fluorescein isothiocyanate (FTIC) isomer, e.g., F0111. In certain embodiments, a high diagnostic score comprises a percentile selected from the vertical line of FIG. 1 which represents the high diagnostic score (e.g., 75%) within a distribution curve of platelet activation measurements observed in a population sample. In still other embodiments, a normal diagnostic score comprises a percentage of activated platelets, which is about 20%, and the high diagnostic score comprises a percentage of activated platelets, which is about 50%, both of which are measured by an anti-fibrinogen antibody, e.g., purified immunoglobulin fraction of rabbit antiserum conjugated with fluorescein isothiocyanate (FTIC) isomer, e.g., F0111. In yet other embodiments, a normal diagnostic score comprises a percentage of activated platelets, which is about 30%, and the high diagnostic score comprises a percentage of activated platelets, which is about 50%, both of which are measured by an anti-fibrinogen antibody, e.g., purified immunoglobulin fraction of rabbit antiserum conjugated with fluorescein isothiocyanate (FTIC) isomer, e.g., F0111. In a specific embodiment, a normal diagnostic score is the median percentage of positive platelets shown in FIG. 2B, i.e., about 35.6% of positive platelets. In other embodiments, a normal diagnostic score is 25%, 30%, 35%, 40%, 45%, or 50% of positive platelets. In yet other embodiments, a high diagnostic score is 55%, 60%, 65%, 70%, 75%, or 80% of positive platelets.

In some embodiments, the normal diagnostic score comprises a percentage of activated platelets, which is about 40%, and the high diagnostic score comprises a percentage of activated platelets, which is about 60%, both of which are measured by an anti-fibrinogen antibody, e.g., purified immunoglobulin fraction of rabbit antiserum conjugated with fluorescein isothiocyanate (FTIC) isomer, e.g., F0111.

In certain embodiments, the antibody specifically binds to a marker such as P-selectin using a specific antibody against P-selectin. For example, an antibody against P-selectin can be selected from CLB-thromb/6, AK4, 1E3, CTB201, P8G6, MAB2154, ab91132, LS-B3578, LS-B3656, LS-C134593, LS-C13963, 10-667-C100, 11-450-C100, 1A-450-T100, 1Y-450-T100, OASA02343, PN IM1315, or any combinations thereof. Other antibodies against P-selectin can also be used for the methods of the invention. In a specific embodiment, the antibody against P-selectin is clone CLB-thromb/6 (available from BECKMAN COULTER®). CLB-Thromb/6 recognises the region located between the lectin and EGF-like domains. The antibody CLB-Thromb/6 was assigned to CD62P during the fourth HLDA Workshop on Human Leucocyte Differentiation Antigens in Vienna, Austria, in 1989 and was studied at the fifth International Workshop on Human Leukocyte Differentiation Antigens in Boston. See Modderman P. W., “CD62 cluster report”, 1989, Leucocyte Typing IV, White Cell Differentiation Antigens. Schlossman, S. F., et al., Eds., Oxford University Press, 1038-1042; and Diacovo, T., et al., “CD62P (P-selectin) cluster report,” 1995, Leucocyte Typing V, White Cell Differentiation Antigens. Schlossman, S. F., et al., Eds., Oxford University Press 1500-1501. AK4, 1E3, CTB201, and P8G6 (catalog: sc-18834) are available from Santa Cruz Biotechnology, Inc. and are monoclonal mouse anti-P-selectin antibodies. MAB2154 is available from EMD Millipore Corporation and is a monoclonal mouse anti-P-selectin antibody. Ab91132 is available from ABCAM® and is a monoclonal mouse anti-P-selectin antibody. LS-B3578 (polyclonal rabbit anti-P-selectin antibody), LS-B3656 (monoclonal mouse anti-P-selectin antibody), LS-C134593 (monoclonal mouse anti-P-selectin antibody), and LS-C13963 (monoclonal mouse anti-P-selectin antibody conjugated to FITC) are available from LifeSpan Biosciences. 10-667-C100, 11-450-C100, 1A-450-T100, and 1Y-450-T100 are available from EXBIO Antibodies and are monoclonal mouse anti-P-selectin antibody. Anti-P-selectin antibodies available from Aviva Systems Biology, e.g., OASA02343, OASA02338, OASA02337, and OASA02339 (polyclonal mouse anti-P-selectin antibody), can also be used for the methods. Other P-selectin antibodies useful for the methods are also available: MHCD6204 (monoclonal mouse anti-P-selectin antibody from Life Technologies); PA1715 (polyclonal rabbit anti-P-selectin antibody from Boster Immunoleader); MCA796GA, MCA2418A488, MCA418, MCA2419 (monoclonal mouse anti-P-selectin antibody available from AbD Serotec); HPA002655 (polyclonal rabbit anti-P-selectin antibody available from Atlas Antibodies); 12-0628-41, 11-0628-41, 11-0628-42, and 12-0626-80 (monoclonal mouse anti-P-selectin antibody available from eBioscience); 304906, 304918, 304912, and 304914 (monoclonal mouse anti-P-selectin antibody available from BioLegend); and BBA34 (monoclonal mouse anti-P-selectin antibody available from R&D Systems). PN IM1315 is available from Beckman Coulter and is an antibody from clone CLB-Thromb/6.

In some embodiments, a normal diagnostic score measured by clone CLB/thromb/6 comprises a percentage of activated platelets, which can be a value above the ones observed in the lower quarter and below the ones observed in the upper quartile. In other embodiments, a high diagnostic score measured by clone CLB/thromb/6 comprises a percentage of activated platelets, which can be in the upper quarter of the observed distribution. In certain embodiments, a normal diagnostic score is the median percentage of positive platelets shown in FIG. 2B.

In certain embodiments, a normal diagnostic score is a percentage of activated platelets (i.e., positive platelets) measured by both an anti-fibrinogen antibody and an anti-P-Selectin antibody, e.g., F0111 and CLB-thromb/6. In some embodiments, a normal diagnostic score is about 20%, about 25%, about 30%, about 35%, about 40%, about 45%, or about 50% of activated platelets measured by an anti-fibrinogen antibody, e.g., F0111, and about 60%, about 65%, about 70%, about 75%, or about 80% of activated platelets measured by an anti-P-Selectin antibody, e.g., CLB/thromb/6. In other embodiments, a normal diagnostic score is the median percentage of the activated platelets in FIG. 2B, i.e., about 85.9% of positive platelets. In some embodiments, a normal diagnostic score is about 80%, 81%, 82%, 83%, 84%, 85%, 86%, 87%, 89%, or 90% of positive cells. In other embodiments, a high diagnostic score is about 90%, 91%, 92%, 93%, 94%, 95%, 96%, 97%, 98%, or 99% of positive platelets.

Other antibodies against fibrinogen and P-selectin, or antibodies against other membrane proteins which change expression level upon activation can also be used to measure platelet activation. Due to the differences in the antibody binding affinity to its antigen, however, the antibodies can be standardized or calibrated by the methods known in the art. See Immunoassay, A Practical Guide, Ed. By Brian Law. Taylor & Francis Library 2005.

### III. PAR4 AGONIST

As used herein, the term “PAR4 agonist” means a peptide comprising one of Formula I, II, III, or IV and fully or partially activating the PAR4 receptor and eliciting signaling events and/or functional responses associated with PAR4 receptor activation. The PAR4 agonist useful to identify a PAR4 agonist responder or a PAR4 antagonist responder comprises an amino acid sequence of Formula I:

Ala-Xaa₁-Pro-Gly-Xaa₂-Leu-Val (Formula I)

wherein,

the amino terminus of the peptide is free or not fused to an amino acid;

Xₐₐ₁ is selected from Tyr and Phe(4-F);

Xₐₐ₂ is selected from Trp(5-OH), (D,L)-Trp(5-Br), D-Trp, Bzt, Tpi, His, Tza, 3-Thi, 3-Fur, His(Bzl), Phe, Tyr, Phe(penta-F), 2-Pya, 3-Pya, 4-Pya, Dpa, 3-Pya(4-Tolyl), Bip(2-Methyl), 1-Naphthyl-Ala, 2-Naphthyl-Ala, Tyr(Bzl) and Styryl-Ala; and

the C-terminus is amidated.

In some embodiments, the PAR4 agonist comprises an amino acid sequence of Formula I and further comprises Lys after Val. In other embodiments, the PAR4 agonist comprises an amino acid sequence of Formula I and further comprises Lys-Asn after Val. In other embodiments, the PAR4 agonist comprises an amino acid sequence of Formula I and further comprises Lys-Asn-Gly after Val.

In other embodiments, the PAR4 agonist further comprises Lys after Val, as shown below in Formula II:

Ala-Xₐₐ₁-Pro-Gly-Xₐₐ₂-Leu-Val-Lys (Formula II)

wherein,

the amino terminus of the peptide is free or not fused to an amino acid;

Xₐₐ₁ is selected from Tyr and Phe(4-F);

Xₐₐ₂ is selected from Trp(5-OH), (D,L)-Trp(5-Br), D-Trp, Bzt, Tpi, His, Tza, 3-Thi, 3-Fur, His(Bzl), Phe, Tyr, Phe(penta-F), 2-Pya, 3-Pya, 4-Pya, Dpa, 3-Pya(4-Tolyl), Bip(2-Methyl), 1-Naphthyl-Ala, 2-Naphthyl-Ala, Tyr(Bzl) and Styryl-Ala; and

the C-terminus is amidated.

In other embodiments, the PAR4 agonist further comprises Lys-Asn after Val, as shown below in Formula III:

Ala-Xaa₁-Pro-Gly-Xaa₂-Leu-Val-Lys-Asn (Formula III)

wherein,

the amino terminus of the peptide is free or not fused to an amino acid;

Xₐₐ₁ is selected from Tyr and Phe(4-F);

Xₐₐ₂ is selected from Trp(5-OH), (D,L)-Trp(5-Br), D-Trp, Bzt, Tpi, His, Tza, 3-Thi, 3-Fur, His(Bzl), Phe, Tyr, Phe(penta-F), 2-Pya, 3-Pya, 4-Pya, Dpa, 3-Pya(4-Tolyl), Bip(2-Methyl), 1-Naphthyl-Ala, 2-Naphthyl-Ala, Tyr(Bzl) and Styryl-Ala; and

the C-terminus is amidated.

In other embodiments, the PAR4 agonist further comprises Lys-Asn-Gly after Val, as shown below in Formula IV:

Ala-Xₐₐ₁-Pro-Gly-Xₐₐ₂-Leu-Val-Lys-Asn-Gly (Formula IV)

wherein,

the amino terminus of the peptide is free or not fused to an amino acid;

Xₐₐ₁ is selected from Tyr and Phe(4-F);

Xₐₐ₂ is selected from Trp(5-OH), (D,L)-Trp(5-Br), D-Trp, Bzt, Tpi, His, Tza, 3-Thi, 3-Fur, His(Bzl), Phe, Tyr, Phe(penta-F), 2-Pya, 3-Pya, 4-Pya, Dpa, 3-Pya(4-Tolyl), Bip(2-Methyl), 1-Naphthyl-Ala, 2-Naphthyl-Ala, Tyr(Bzl) and Styryl-Ala; and

the C-terminus is amidated.

In some embodiments Xₐₐ₁ is Phe(4-F). In other embodiments, Xₐₐ₂ is Trp. In some embodiments, the peptide consists essentially of or consists of the amino acid sequence. In other embodiments, the C-terminus of the peptide is amidated. In some embodiments, the amino acid sequence further comprises Lys after Val as shown in Formula I. In other embodiments, the amino acid sequence further comprises Lys-Asn after Val as shown in Formula I. In other embodiments, the amino acid sequence further comprises Lys-Asn-Gly after Val as shown in Formula I. In some embodiments, the peptide activates a PAR4 receptor. In other embodiments, PAR4 receptor activation is measured by a platelet aggregation assay, a FLIPR assay, or both. In some embodiments, PAR4 receptor activation is higher than the PAR4 receptor activation by the peptide consisting of SEQ ID NO: 1. For example, PAR4 receptor activation can be at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, or at least about 110-fold higher than PAR4 receptor activation by the peptide consisting of SEQ ID NO: 1.

Exemplary PAR4 agonist peptides are SEQ ID NOS: 1-8, 12-16 and 18-34, shown in Table 4, wherein the N-termini of the sequences are free and the C-termini of the sequences are amidated. In Table 4, the numbers 1-10 at the top of the chart reflect the position of the amino acid within the peptide, with position 1 starting at the N-terminus.

TABLE 4
PAR4 AGONISTS

| SEQ | | | | | | | | | | |
|---|---|---|---|---|---|---|---|---|---|---|
| ID | | | | | | | | | | |
| NO. | 1 | 2 | 3 | 4 | 5 | 6 | 7 | 8 | 9 | 10 |
| 1 | A | Y | P | G | K | F | | | | |
| 2 | A | Y | P | G | W | L | V | K | N | G |
| 3 | A | Phe(4-F) | P | G | W | L | V | K | N | G |
| 4 | A | Phe(4-F) | P | G | W | L | V | K | N | |
| 5 | A | Phe(4-F) | P | G | W | L | V | K | | |
| 6 | A | Phe(4-F) | P | G | W | L | V | | | |
| 7 | A | Phe(4-F) | P | G | W | L | | | | |
| 8 | A | Phe(4-F) | P | G | W | | | | | |
| 9 | A | Phe(4-F) | P | G | | | | | | |
| 10 | A | Y | P | G | | | | | | |
| 11 | A | Y | P | G | Q | V | C | A | N | D |
| 12 | A | Phe(4-F) | P | G | Trp(5-OH) | L | V | | | |
| 13 | A | Phe(4-F) | P | G | (D,L)- | L | V | | | |
| | | | | | Trp(5-Br) | | | | | |
| 14 | A | Phe(4-F) | P | G | D-Trp | L | V | | | |
| 15 | A | Phe(4-F) | P | G | Bzt | L | V | | | |
| 16 | A | Phe(4-F) | P | G | Tpi | L | V | | | |
| 17 | A | Phe(4-F) | P | G | H | L | V | | | |
| 18 | A | Phe(4-F) | P | G | Tza | L | V | | | |
| 19 | A | Phe(4-F) | P | G | 3-Thi | L | V | | | |
| 20 | A | Phe(4-F) | P | G | 3-Fur | L | V | | | |
| 21 | A | Phe(4-F) | P | G | His(Bzl) | L | V | | | |
| 22 | A | Phe(4-F) | P | G | F | L | V | | | |
| 23 | A | Phe(4-F) | P | G | Y | L | V | | | |
| 24 | A | Phe(4-F) | P | G | Phe(penta-F) | L | V | | | |
| 25 | A | Phe(4-F) | P | G | 2-Pya | L | V | | | |
| 26 | A | Phe(4-F) | P | G | 3-Pya | L | V | | | |
| 27 | A | Phe(4-F) | P | G | 4-Pya | L | V | | | |
| 28 | A | Phe(4-F) | P | G | Dpa | L | V | | | |
| 29 | A | Phe(4-F) | P | G | 3-Pya(4-Tolyl) | L | V | | | |
| 30 | A | Phe(4-F) | P | G | Bip(2-Methyl) | L | V | | | |
| 31 | A | Phe(4-F) | P | G | 1-Naphthyl-Ala | L | V | | | |
| 32 | A | Phe(4-F) | P | G | 2-Naphthyl-Ala | L | V | | | |
| 33 | A | Phe(4-F) | P | G | Tyr(Bzl) | L | V | | | |
| 34 | A | Phe(4-F) | P | G | Styryl-Ala | L | V | | | |

In one embodiment, the PAR4 agonist comprises a peptide selected from SEQ ID NOS: 2-7, 12, 13, 15, 18-24 and 26-34. In another embodiments, the PAR4 agonist comprises the peptide of SEQ ID NO: 3. In other embodiments, the PAR4 agonist consists of the amino acid sequence of SEQ ID NO: 2. In yet other embodiments, the PAR4 agonist consists of the amino acid sequence of SEQ ID NO: 3. In some embodiments, the PAR4 agonist consists of the amino acid sequence of SEQ ID NO: 4. In other embodiments, the PAR4 agonist consists of the amino acid sequence of SEQ ID NO: 5. In some embodiments, the PAR4 agonist consists of the amino acid sequence of SEQ ID NO: 6. In some embodiments, the PAR4 agonist consists of the amino acid sequence of SEQ ID NO: 7. In other embodiments, the PAR4 agonist consists of the amino acid sequence of SEQ ID NO: 12. In some embodiments, the PAR4 agonist consists of the amino acid sequence of SEQ ID NO: 13. In other embodiments, the PAR4 agonist consists of the amino acid sequence of SEQ ID NO: 15. In some embodiments, the PAR4 agonist consists of the amino acid sequence of SEQ ID NO: 18. In other embodiments, the PAR4 agonist consists of the amino acid sequence of SEQ ID NO: 19. In some embodiments, the PAR4 agonist consists of the amino acid sequence of SEQ ID NO: 20. In some embodiments, the PAR4 agonist peptide consists of the amino acid sequence of SEQ ID NO: 21. In other embodiments, the PAR4 agonist consists of the amino acid sequence of SEQ ID NO: 22. In some embodiments, the PAR4 agonist consists of the amino acid sequence of SEQ ID NO: 23. In other embodiments, the PAR4 agonist consists of the amino acid sequence of SEQ ID NO: 24. In some embodiments, the PAR4 agonist consists of the amino acid sequence of SEQ ID NO: 26. In other embodiments, the PAR4 agonist consists of the amino acid sequence of SEQ ID NO: 27. In some embodiments, the PAR4 agonist consists of the amino acid sequence of SEQ ID NO: 28. In other embodiments, the PAR4 agonist consists of the amino acid sequence of SEQ ID NO: 29. In some embodiments, the PAR4 agonist consists of the amino acid sequence of SEQ ID NO: 30. In other embodiments, the PAR4 agonist consists of the amino acid sequence of SEQ ID NO: 31. In some embodiments, the PAR4 agonist consists of the amino acid sequence of SEQ ID NO: 32. In other embodiments, the PAR4 agonist consists of the amino acid sequence of SEQ ID NO: 33. In some embodiments, the PAR4 agonist consists of the amino acid sequence of SEQ ID NO: 34.

In some embodiments, a PAR4 agonist has IC₅₀ in the FLIPR Assay of 5 μM or less, 500 nM or less, or 10 nM or less. Examples of such PAR4 agonists are those reported in the specific Working Examples herein. Activity data for a number of these compounds is presented in the table of Example 1.

In some embodiments, a PAR4 agonist has an ED₅₀<100 μM. In other embodiments, a PAR4 agonist has an ED₅₀<10 μM. Exemplary assays for measuring PAR4 agonist activity include, but are not limited to, the platelet aggregation assay described in Example 1 and the FLIPR assay described in Example 1.

An amino acid includes a compound represented by the general structure:

where R and R′ are as discussed herein. Unless otherwise indicated, the term “amino acid” as employed herein, alone or as part of another group, includes, without limitation, an amino group and a carboxyl group linked to the same carbon, referred to as “α” carbon, where R and/or R′ can be a natural or an un-natural side chain, including hydrogen. The absolute “S” configuration at the “α” carbon is commonly referred to as the “L” or “natural” configuration, with the exception of L-Cysteine, which possesses an absolute “R” configuration. In the case where both the “R” and the “R′” (prime) substituents equal hydrogen, the amino acid is glycine and is not chiral. The amino acids recited herein are in the “L” configuration unless noted otherwise.

Amino acids can be referred to herein by either their commonly known three-letter symbols or by the one-letter symbols recommended by the IUPAC-IUB Biochemical Nomenclature Commission. A PAR4 agonist for the present invention can include naturally encoded amino acids (common amino acids) as well as non-naturally encoded amino acids. A “non-naturally encoded amino acid” refers to an amino acid that is not one of the 20 common amino acids or pyrrolysine or selenocysteine. Other terms that can be used synonymously with the term “non-naturally encoded amino acid” are “non-natural amino acid,” “unnatural amino acid,” “non-naturally-occurring amino acid,” and variously hyphenated and non-hyphenated versions thereof. Exemplary non-naturally encoded amino acids that can be present in the PAR4 agonist peptides are shown below.

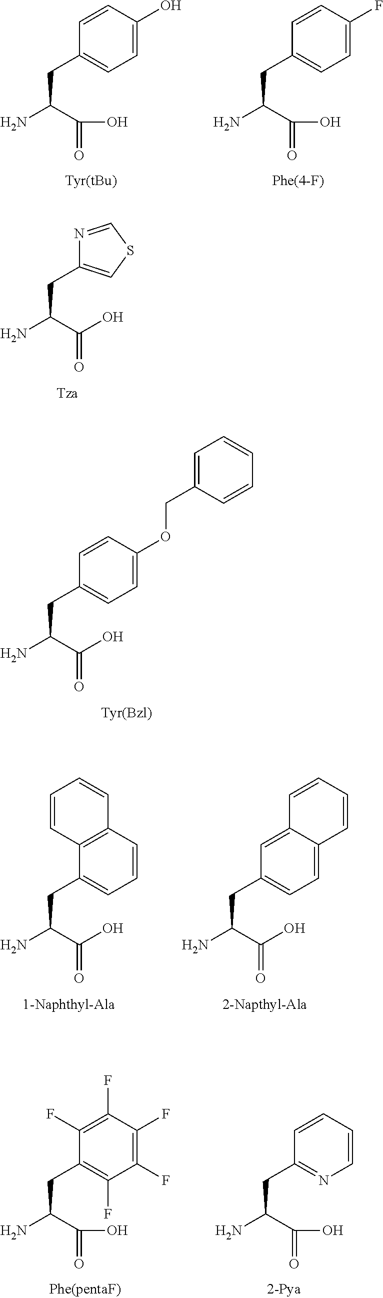

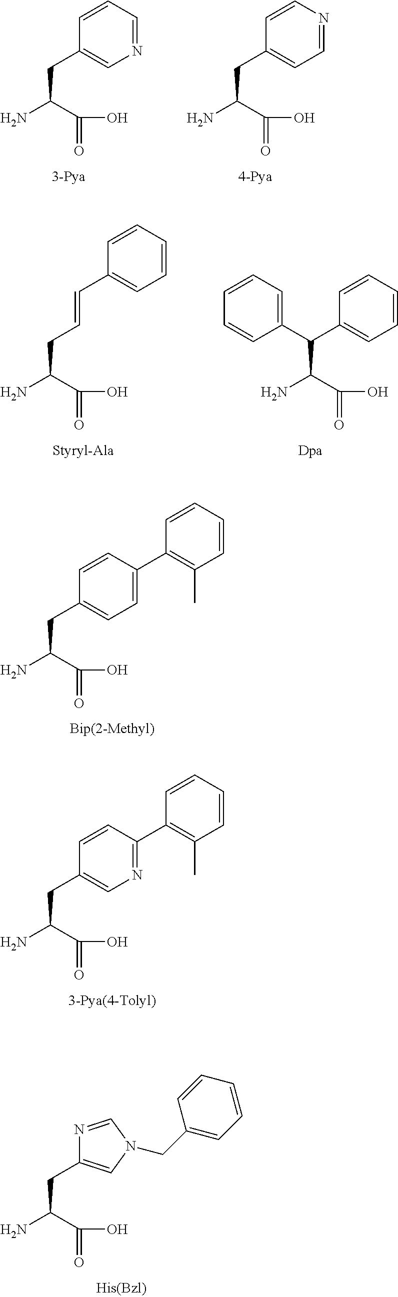

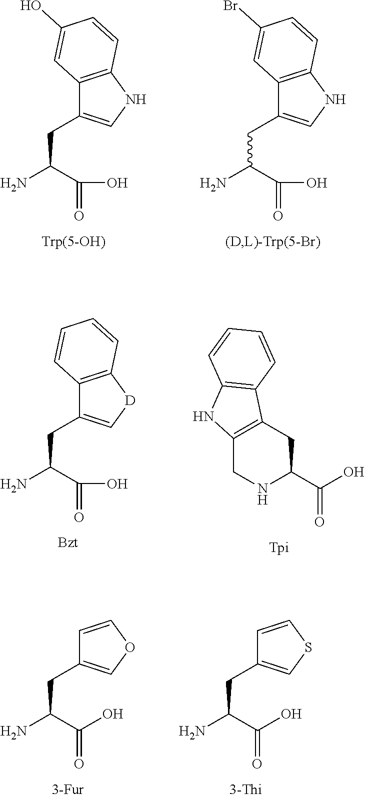

The PAR4 agonists disclosed herein show improved affinity for the PAR-4 receptor and can be used as agonists to activate the PAR-4 receptor in PAR4 receptor assays.

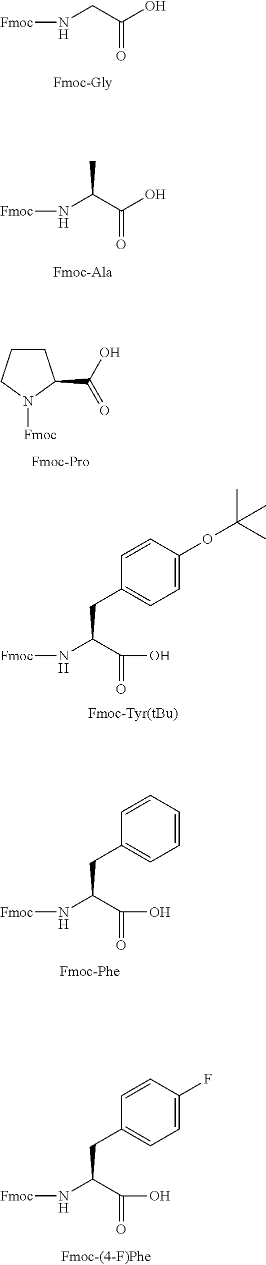

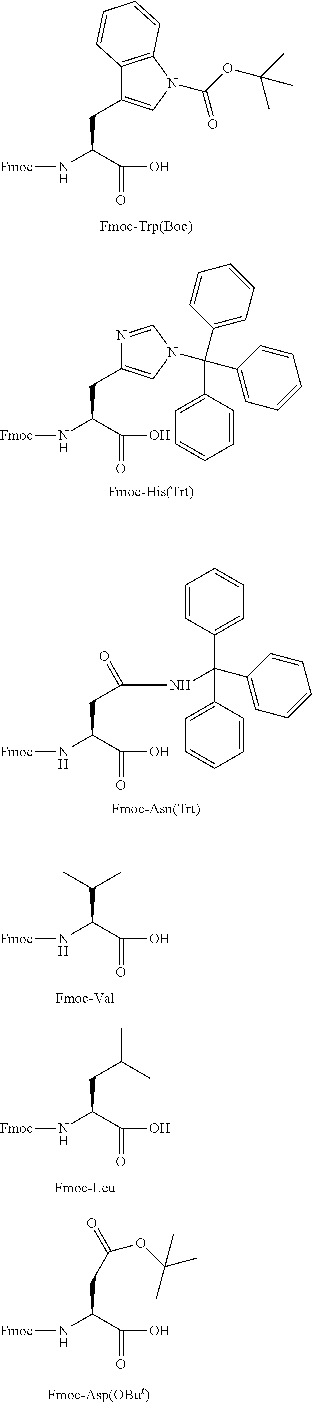

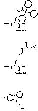

### IV. PAR4 ANTAGONIST

In one embodiment, a PAR4 antagonist that is useful in the present invention are those compounds, stereoisomers, tautomers, salts, solvates or prodrugs thereof, disclosed in International Patent Application Filing Number PCT/US2013/037884, which is herein incorporated by reference in its entirety.

In another embodiment, a PAR4 antagonist that is useful in the present invention are those compounds, stereoisomers, tautomers, salts, solvates or prodrugs thereof, disclosed in International Patent Application Filing Number PCT/US2013/037956, which is herein incorporated by reference in its entirety.

In yet another embodiment, PAR4 antagonist that is useful in the present invention are those compounds, stereoisomers, tautomers, salts, solvates or prodrugs thereof, disclosed in International Patent Application Filing Number PCT/US2013/037892, which is herein incorporated by reference in its entirety.

In one embodiment, PAR4 antagonist that is useful in the present invention are compounds, stereoisomers, tautomers, salts, solvates or prodrugs thereof, of Formula V having the structure:

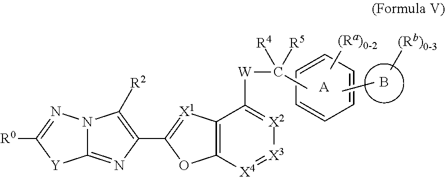

or a stereoisomer, tautomer, pharmaceutically acceptable salt, solvate or prodrug ester thereof,
wherein:

W is O or S;

R⁰ is R¹ or R^{1a};

Y is S or —CR⁸═CR⁹—;

R¹ is independently selected from the group consisting of:
halo,
C₁-C₄ alkyl,
C₂-C₃ alkenyl,
C₂-C₃ alkynyl,
C₃-C₄ cycloalkyl,
C₁-C₄ alkoxy,
C₁-C₂ alkoxy-C₁-C₂ alkyl,
tetrahydrofuran-2-yl;
C₁-C₄ alkylthio,
C₁-C₄ alkylNH—,
(C₁-C₄ alkyl)₂N—,
halo-C₁-C₂-alkyl, which contains 1 to 5 halogens, where halo is F or Cl,
halo-C₃-C₄ cycloalkyl,
halo-C₁-C₂ alkoxy, and
halo-C₁-C₂ alkylthio;

R^{1a} is independently selected from the group consisting of:
H,
halo,
C₁-C₄ alkyl,
C₂-C₃ alkenyl,
C₂-C₃ alkynyl,
C₃-C₄ cycloalkyl,
C₁-C₄ alkoxy,
C₁-C₂ alkoxy-C₁-C₂ alkyl,
tetrahydrofuran-2-yl;
C₁-C₄ alkylthio,
C₁-C₄ alkylNH—,
(C₁-C₄ alkyl)₂N—,
halo-C₁-C₂-alkyl, which contains 1 to 5 halogens, where halo is F or Cl,
halo-C₃-C₄ cycloalkyl,
halo-C₁-C₂ alkoxy, and
halo-C₁-C₂ alkylthio;

R⁸ and R⁹ are independently selected from the group consisting of:
H,
halo,
C₁-C₄ alkyl,
C₁-C₄ alkoxy,
halo-C₁-C₂ alkyl,
halo-C₁-C₂ alkoxy,
CN, and
OH;
   provided that at least one of R^{1a}, R⁸ and R⁹ is other than H;

R² is selected from the group consisting of:
H,
halo,
C₁-C₄ alkyl,
C₁-C₄ alkoxy, and
cyano;

X¹ is selected from the group consisting of CH, N or CR¹⁰;

X², X³ and X⁴ are independently selected from CR³ or N;

R³ is selected from the group consisting of H, C₁-C₄ alkyl, C₂-C₄ alkenyl, C₂-C₄ alkynyl, C₁-C₄ alkoxy, C₁-C₄ alkylthio, halo, OH, CN, OCF₃, OCHF₂, OCH₂F, C₁-C₂-alkoxy-C₁-C₂-alkoxy, halo-C₁-C₃-alkyl, which contains 1 to 5 halogens, benzyloxy substituted by 0 to 3 groups independently selected from the group consisting of halo, C₁-C₄ alkoxy, C₁-C₄ alkyl, cyclopropyl, CF₃, OCF₃, 5- or 6-membered heteroaryl, OH, OCHF₂, di-C₁-C₄-alkylamino, and cyano, and —(CH₂)ₙ¹-phenyl substituted by 0 to 3 groups independently selected from the group consisting of halo, C₁-C₄ alkoxy, C₁-C₄ alkyl, cyclopropyl, CF₃, OCF₃, 5- or 6-membered heteroaryl, OH, OCHF₂, di-C₁-C₄-alkylamino, and cyano;

R⁴ and R⁵ are independently selected from H, C₁-C₆ alkyl, halo-C₁-C₃ alkyl, hydroxy-C₁-C₃ alkyl, and C₁-C₃ alkoxy-C₁-C₃ alkyl; or R⁴ and R⁵ can be taken together with the carbon to which they are attached to form a C₃-C₇ cycloalkyl ring;

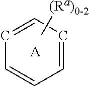

is phenyl or a 6-membered heteroaryl ring, at least one ring member of which is a nitrogen, which

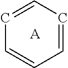

ring is substituted with 0 to 2 R^{a} groups;

B is selected from the group consisting of a C₆-C₁₀ aryl, a 5- to 10-membered heteroaryl, a 4- to 10-membered heterocyclyl containing carbon atoms and 1 to 4 additional heteroatoms selected from N, O, and S, and a C₃-C₈ cycloalkyl which can contain unsaturation, all of which are substituted by 0 to 3 R^{b} groups;

R^{a}, at each occurrence, is independently selected from the group consisting of H, halo, halo-C₁-C₄ alkoxy, OH, CN, NO₂, NR⁶R⁷, COOH, C₁-C₄ alkoxy, C₁-C₄ alkylthio, C₁-C₄ alkoxycarbonyl, (C═O)NR⁶R⁷, C₁-C₄ alkoxy-C₁-C₄ alkoxy, C₁-C₄ alkylsulfonyl, C₁-C₄ alkylsulfinyl, S(═O)₂NR⁶R⁷, and C₁-C₅ alkyl substituted by 0 to 7 groups independently selected from halo, CF₃, OCF₃, OH, hydroxy-C₁-C₄-alkyl, C₁-C₄ alkoxy, C₁-C₄ alkoxy-C₁-C₄ alkoxy, di-C₁-C₄-alkylaminophenyl-C₁-C₄-alkyl, (di-C₁-C₄-alkoxy-C₁-C₄-alkyl)-C₁-C₄-alkyl, di-C₁-C₄-alkylamino, C₃-C₆-cycloalkyl, phenyl, and C₁-C₄ alkylthio;

R^{b}, at each occurrence, is independently selected from the group consisting of H, halo, halo-C₁-C₄ alkoxy, OH, CN, NO₂, NR⁶R⁷, COOH, C₁-C₄ alkoxy, C₁-C₄ alkylthio, C₁-C₄ alkoxycarbonyl, (C═O)NR⁶R⁷, C₁-C₄ alkoxy-C₁-C₄ alkoxy, C₁-C₄ alkylsulfonyl, C₁-C₄ alkylsulfinyl, S(═O)₂NR⁶R⁷, N(R¹³)(C═O)NR⁶R⁷, N(R¹³)(C═O)OR¹⁴, N(R¹³)(C═O)R¹⁴, NR¹³S(O)R¹⁴, NR¹³SO₂R¹⁴, O(C═O)NR⁶R⁷, O(C═O)OR¹⁴, O(C═O)R¹⁴, (C═O)OR¹⁴, C₆-C₁₀ aryl, 5-6-membered heteroaryl, 4- to 10-membered heterocyclyloxy and C₁-C₅ alkyl substituted by 0 to 7 groups independently selected from halo, CF₃, OCF₃, OH, hydroxy-C₁-C₄-alkyl, C₁-C₄ alkoxy, C₁-C₄ alkoxy-C₁-C₄ alkoxy, di-C₁-C₄-alkylaminophenyl-C₁-C₄-alkyl, (di-C₁-C₄-alkoxy-C₁-C₄-alkyl)-C₁-C₄-alkyl, di-C₁-C₄-alkylamino, C₃-C₆-cycloalkyl, phenyl, C₁-C₄-alkoxyphenyl-C₁-C₄-alkoxy, 4- to 10-membered heterocyclyloxy and C₁-C₄ alkylthio;

R⁶ and R⁷ are independently, at each occurrence, selected from the group consisting of:
H,
C₁-C₄ alkyl,
halo-C₁-C₄-alkyl,
C₁-C₄ alkyleneoxy-C₁-C₄-alkylene,
C₂-C₄ alkenyl,
C₂-C₄ alkynyl,
—(CR¹⁴R¹⁴)ₙ¹-phenyl substituted by 0 to 3 groups independently selected from the group consisting of halo, C₁-C₄ alkoxy, C₁-C₄ alkyl, cyclopropyl, CF₃, OCF₃, 5- or 6-membered heteroaryl, OH, OCHF₂, di-C₁-C₄-alkylamino, and cyano,
—(CHR¹³)ₙ¹—C₃-C₆-cycloalkyl substituted by 0 to 3 groups independently selected from the group consisting of halo, CF₃, OCF₃, 5- or 6-membered heteroaryl, OH, hydroxy-C₁-C₄-alkyl, and C₁-C₄ alkyl,
—(CHR¹³)ₙ¹-4- to 10-membered-heterocyclyl substituted by 0 to 3 groups independently selected from the group consisting of halo, CF₃, OCF₃, 5- or 6-membered heteroaryl, OH, oxo, hydroxy-C₁-C₄-alkyl, and C₁-C₄ alkyl,
—(CHR¹³)ₙ¹-5- to 10-membered-heteroaryl substituted by 0 to 3 groups independently selected from the group consisting of halo, CF₃, OCF₃, 5- or 6-membered heteroaryl, OH, hydroxy-C₁-C₄-alkyl, and C₁-C₄ alkyl,
di-C₁-C₄-alkylamino-C₁-C₄-alkyl,
C₁-C₄-alkylcarbonylamino-C₁-C₄-alkyl,
di-C₁-C₄-alkoxy-C₁-C₄-alkyl,
di-C₁-C₄-alkylaminophenyl,
hydroxy-C₁-C₄-alkyl,
cyano-C₁-C₄-alkyl,
C₁-C₄-alkoxy-C₁-C₄-alkyl,
C₁-C₄-alkoxycarbonyl-C₁-C₄-alkyl,
C₁-C₄-alkoxycarbonyl,
C₁-C₄-alkylcarbonyl,
phenylcarbonyl;
C₁-C₄-alkoxycarbonylamino-C₁-C₄-alkylcarbonyl,
di-C₁-C₄-alkylamino-C₁-C₄-alkylcarbonyl,
amino-C₁-C₄-alkylcarbonyl,
4- to 10-membered-heterocyclyl-carbonyl, and
   alternatively, R⁶ and R⁷, when attached to the same nitrogen, combine to form a 4- to 8-membered heterocyclic ring containing carbon atoms substituted by 0 to 3 groups independently selected from the group consisting of halo, CF₃, CHF₂, OCF₃, OCHF₂, OCH₂F, 5- or 6-membered heteroaryl, OH, oxo, hydroxy-C₁-C₄-alkyl, C₁-C₄ alkyl and C₁-C₄ alkoxy, and 0 to 2 additional heteroatoms selected from N, NR¹³, O and S(O)ₚ;

R¹³ is independently, at each occurrence, selected from the group consisting of H, C₁-C₆ alkyl and —(CH₂)phenyl;

R¹⁴ is independently, at each occurrence, selected from the group consisting of H, C₁-C₆ alkyl, halo-C₁-C₄-alkyl, C₁-C₄-alkoxycarbonylamino, (C₆-C₁₀ arylcarbonylamino), (a 5- to 10-membered heteroarylcarbonylamino) and —(CH₂)ₙ¹ phenyl substituted by 0 to 3 groups independently selected from the group consisting of halo, C₁-C₄ alkoxy, C₁-C₄ alkyl, cyclopropyl, CF₃, OCF₃, 5- or 6-membered heteroaryl, OH, OCHF₂, di-C₁-C₄-alkylamino, and cyano,

R¹⁰ is selected from the group consisting of C₁-C₄ alkyl, halo, C₁-C₄ alkoxy, and halo-C₁-C₂-alkyl, which contains 1 to 5 halogens, where halo is F or Cl;

n¹, at each occurrence, is selected from 0, 1, 2, 3, 4 or 5; and

p, at each occurrence, is selected from 0, 1 and 2.

In another embodiment, PAR4 antagonist that can be useful in the present invention are compounds, stereoisomers, tautomers, salts, solvates or prodrugs thereof, of Formula VI having the structure:

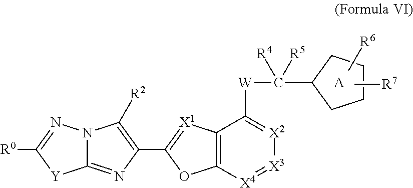

wherein:

W is O or S;

R⁰ is R¹ or R^{1a};

Y is S or —CR⁸═CR⁹—;

R¹ is independently selected from the group consisting of:
halo,
C₁-C₄ alkyl,
C₂-C₃ alkenyl,
C₂-C₃ alkynyl,
C₃-C₄ cycloalkyl,
C₁-C₄ alkoxy,
C₁-C₂ alkoxy-C₁-C₂ alkyl,
tetrahydrofuran-2-yl;
C₁-C₄ alkylthio,
C₁-C₄ alkylNH—,
(C₁-C₄ alkyl)₂N—,
halo-C₁-C₂-alkyl, which contains 1 to 5 halogens, where halo is F or Cl,
halo-C₃-C₄ cycloalkyl,
halo-C₁-C₂ alkoxy, and
halo-C₁-C₂ alkylthio;

R^{1a} is independently selected from the group consisting of:
H,
halo,
C₁-C₄ alkyl,
C₂-C₃ alkenyl,
C₂-C₃ alkynyl,
C₃-C₄ cycloalkyl,
C₁-C₄ alkoxy,
C₁-C₂ alkoxy-C₁-C₂ alkyl,
tetrahydrofuran-2-yl;
C₁-C₄ alkylthio,
C₁-C₄ alkylNH—,
(C₁-C₄ alkyl)₂N—,
halo-C₁-C₂-alkyl, which contains 1 to 5 halogens, where halo is F or Cl,
halo-C₃-C₄ cycloalkyl,
halo-C₁-C₂ alkoxy, and
halo-C₁-C₂ alkylthio;

R⁸ and R⁹ are independently selected from the group consisting of:
H,
halo,
C₁-C₄ alkyl,
C₁-C₄ alkoxy,
halo-C₁-C₂ alkyl,
halo-C₁-C₂ alkoxy,
CN, and
OH;
   provided that at least one of R^{1a}, R⁸ and R⁹ is other than H;

R² is selected from the group consisting of:
H,
halo,
C₁-C₄ alkyl,
C₁-C₄ alkoxy, and
cyano;

X¹ is selected from the group consisting of CH, N or CR¹⁰;

X², X³ and X⁴ are independently selected from CR³ or N;

R³ is selected from the group consisting of H, C₁-C₄ alkyl, C₂-C₄ alkenyl, C₂-C₄ alkynyl, C₁-C₄ alkoxy, C₁-C₄ alkylthio, halo, OH, CN, OCF₃, OCHF₂, OCH₂F, C₁-C₂-alkoxy-C₁-C₂-alkoxy, halo-C₁-C₃-alkyl, which contains 1 to 5 halogens, benzyloxy substituted by 0 to 3 groups independently selected from the group consisting of halo, C₁-C₄ alkoxy, C₁-C₄ alkyl, cyclopropyl, CF₃, OCF₃, 5- or 6-membered heteroaryl, OH, OCHF₂, di-C₁-C₄-alkylamino, and cyano, and —(CH₂)ₙ¹-phenyl substituted by 0 to 3 groups independently selected from the group consisting of halo, C₁-C₄ alkoxy, C₁-C₄ alkyl, cyclopropyl, CF₃, OCF₃, 5- or 6-membered heteroaryl, OH, OCHF₂, di-C₁-C₄-alkylamino, and cyano;

R⁴ and R⁵ are independently selected from H and C₁-C₆ alkyl, or R⁴ and R⁵ can be taken together with the carbon to which they are attached to form a C₃-C₇ cycloalkyl ring;

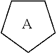

is a 5-membered heteroaryl ring containing at least one O, N or S atom;

R⁶ is selected from the group consisting of H, halo, OCF₃, OCHF₂, OH, CN, NO₂, NR¹¹R¹², COOH, C₁-C₄ alkoxycarbonyl, (C═O)NR^{1A}R¹², C₁-C₄ alkylsulfonyl, S(═O)₂NR¹¹R¹², and C₁-C₅ alkyl substituted by 0 to 7 groups independently selected from halo, CF₃, OCF₃, OH, hydroxy-C₁-C₄-alkyl, C₁-C₄ alkoxy, C₁-C₄ alkoxy-C₁-C₄ alkoxy, di-C₁-C₄-alkylaminophenyl-C₁-C₄-alkyl, (di-C₁-C₄-alkoxy-C₁-C₄-alkyl)-C₁-C₄-alkyl, di-C₁-C₄-alkylamino, C₃-C₆-cycloalkyl, and C₁-C₄ alkylthio, or

R⁶ is B-D-, where D is a linker, which is selected from:
a single bond,
—O—,
—S—,

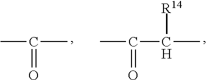

C₁-C₄ alkylene substituted by 0 to 4 groups independently selected from halo or OH,
C₁-C₄ alkyleneoxy,
C₁-C₄ alkylenethio,
C₁-C₄ alkyleneoxy-C₁-C₄-alkylene,
C₁-C₄-alkylenethio-C₁-C₄-alkylene,
—S—C₁-C₄-alkylene,
—O—C₁-C₄-alkylene,
C₂-C₆ alkenylene, and

B is selected from the group consisting of:
C₆-C₁₀ aryl substituted by 0 to 3 groups independently selected from the group consisting of halo, C₁-C₄ alkoxy, halo-C₁-C₄ alkoxy, C₁-C₄ alkyl, halo-C₁-C₄ alkyl, C₃-C₆ cycloalkyl, cyano, nitro, NR¹¹R¹², OH, C₁-C₄-alkylcarbonyloxy-C₁-C₄-alkyl, hydroxy-C₁-C₄-alkyl, cyano-C₁-C₄-alkyl, COOR¹⁴, SO₂R¹⁴, (C═O)NR¹¹R¹², SO₂NR¹¹R¹², N(R¹³)(C═O)NR¹¹R¹², N(R¹³)(C═O)OR¹⁴, N(R¹³)(C═O)R¹⁴, NR¹³S(O)R¹⁴, NR¹³SO₂R¹⁴, O(C═O)NR¹¹R¹², O(C═O)OR¹⁴, O(C═O)R¹⁴, (C═O)OR¹⁴, and 5-6-membered heteroaryl,
5- to 10-membered heteroaryl substituted by 0 to 3 groups independently selected from the group consisting of halo, C₁-C₄ alkoxy, halo-C₁-C₄ alkoxy, C₁-C₄ alkyl, halo-C₁-C₄ alkyl, C₃-C₆ cycloalkyl, cyano, nitro, NR¹¹R¹², OH, C₁-C₄-alkylcarbonyloxy-C₁-C₄-alkyl, hydroxy-C₁-C₄-alkyl, COOR¹⁴, SO₂R¹⁴, (C═O)NR¹¹R¹², SO₂NR¹¹R¹², N(R¹³)(C═O)NR¹¹R¹², N(R¹³)(C═O)OR¹⁴, N(R¹³)(C═O)R¹⁴, NR¹³S(O)R¹⁴, NR¹³SO₂R¹⁴, O(C═O)NR¹¹R¹², O(C═O)OR¹⁴, O(C═O)R¹⁴, (C═O)OR¹⁴, 5-6-membered heteroaryl, and (CH₂)phenyl,
4- to 10-membered heterocyclyl containing carbon atoms and 1 to 2 additional heteroatoms selected from N, O, and S, and substituted by 0 to 3 groups independently selected from the group consisting of halo, oxo, —(CHR¹³)ₙ¹-5- or 6-membered heteroaryl substituted by 0 to 3 groups independently selected from the group consisting of halo, C₁-C₄ alkoxy, C₁-C₄ alkyl, cyclopropyl, CF₃, OCF₃, and CF₂CH₃; NR¹³S(O)R¹⁴, NR¹³SO₂R¹⁴, —(CHR¹³)ₙ¹-4- to 10-membered-heterocyclyl substituted by 0 to 3 groups independently selected from the group consisting of halo, C₁-C₄ alkoxy, C₁-C₄ alkyl, cyclopropyl, CF₃, OCF₃, and CF₂CH₃; OH, hydroxy-C₁-C₄-alkyl, C₁-C₄ alkoxy, halo-C₁-C₄ alkoxy, di-C₁-C₄-alkylamino-C₁-C₄-alkyl, NR₁₁R₁₂, cyano, C₁-C₄ alkyl, halo-C₁-C₄ alkyl, C₃-C₆ cycloalkyl, C₁-C₄-alkoxy-C₁-C₄-alkyl, C₃-C₆ cycloalkyl-C₁-C₄-alkylcarbonyl, C₆-C₁₀ arylcarbonyl, C₁-C₄-alkylcarbonyloxy-C₁-C₄-alkyl, COOR¹⁴, SO₂R¹⁴, (C═O)NR¹¹R¹², SO₂NR¹¹R¹², N(R¹³)(C═O)NR¹¹R¹², N(R¹³)(C═O)OR¹⁴, N(R¹³)(C═O)R¹⁴, O(C═O)NR¹¹R¹², O(C═O)OR¹⁴, O(C═O)R¹⁴, (C═O)OR¹⁴, and C₆-C₁₀ aryl substituted by 0 to 3 groups independently selected from the group consisting of halo, C₁-C₄ alkoxy, C₁-C₄ alkyl, cyclopropyl, C₁-C₄-alkoxycarbonyl, (C═O)NR¹¹R¹², CF₃, OCF₃, and CF₂CH₃;
C₃-C₆ cycloalkyl which can contain unsaturation, substituted by 0 to 3 groups independently selected from the group consisting of halo, CF₃, OCF₃, 5- or 6-membered heteroaryl, OH, oxo, hydroxy-C₁-C₄-alkyl, C₆-C₁₀ aryl, COOH, oxo, C₁-C₄-alkoxycarbonyl, (C═O)NR¹¹R¹², NR¹¹R¹² and C₁-C₄ alkyl; and
C₅-C₁₁ spirocycloalkyl which can contain unsaturation and optionally containing 1 to 3 heteroatoms selected from O, N or S and substituted by 0 to 3 groups independently selected from the group consisting of halo, CF₃, OCF₃, 5- or 6-membered heteroaryl, OH, hydroxy-C₁-C₄-alkyl, C₆-C₁₀ aryl, and C₁-C₄ alkyl;

R¹¹ and R¹² are independently, at each occurrence, selected from the group consisting of:
H,
C₁-C₄ alkyl,
halo-C₁-C₄-alkyl,
C₂-C₄ alkenyl,
C₂-C₄ alkynyl,
—(CR¹⁴R¹⁴)ₙ¹-phenyl substituted by 0 to 3 groups independently selected from the group consisting of halo, C₁-C₄ alkoxy, C₁-C₄ alkyl, cyclopropyl, CF₃, OCF₃, 5- or 6-membered heteroaryl, OH, OCHF₂, di-C₁-C₄-alkylamino, and cyano,
—(CHR¹³)ₙ¹—C₃-C₆-cycloalkyl substituted by 0 to 3 groups independently selected from the group consisting of halo, CF₃, OCF₃, 5- or 6-membered heteroaryl, OH, hydroxy-C₁-C₄-alkyl, and C₁-C₄ alkyl,
—(CHR¹³)ₙ¹-4- to 10-membered-heterocyclyl substituted by 0 to 3 groups independently selected from the group consisting of halo, CF₃, OCF₃, 5- or 6-membered heteroaryl, OH, oxo, hydroxy-C₁-C₄-alkyl, and C₁-C₄ alkyl,
—(CHR¹³)ₙ¹-5- to 10-membered-heteroaryl substituted by 0 to 3 groups independently selected from the group consisting of halo, CF₃, OCF₃, 5- or 6-membered heteroaryl, OH, hydroxy-C₁-C₄-alkyl, and C₁-C₄ alkyl,
di-C₁-C₄-alkylamino-C₁-C₄-alkyl, C₁-C₄-alkylcarbonylamino-C₁-C₄-alkyl,
di-C₁-C₄-alkoxy-C₁-C₄-alkyl, di-C₁-C₄-alkylaminophenyl,
hydroxy-C₁-C₄-alkyl,
cyano-C₁-C₄-alkyl,
C₁-C₄-alkoxy-C₁-C₄-alkyl,
C₁-C₄-alkoxycarbonyl-C₁-C₄-alkyl,
C₁-C₄-alkoxycarbonyl,
C₁-C₄-alkylcarbonyl,
phenylcarbonyl;
C₁-C₄-alkoxycarbonylamino-C₁-C₄-alkylcarbonyl,
di-C₁-C₄-alkylamino-C₁-C₄-alkylcarbonyl,
amino-C₁-C₄-alkylcarbonyl,
4- to 10-membered-heterocyclyl-carbonyl, and
   alternatively, R¹¹ and R¹², when attached to the same nitrogen, combine to form a 4- to 8-membered heterocyclic ring containing carbon atoms substituted by 0 to 3 groups independently selected from the group consisting of halo, CF₃, CHF₂, OCF₃, OCHF₂, OCH₂F, 5- or 6-membered heteroaryl, OH, oxo, hydroxy-C₁-C₄-alkyl, C₁-C₄ alkyl and C₁-C₄ alkoxy, and 0 to 2 additional heteroatoms selected from N, NR¹³, O and S(O)ₚ;

R¹³ is independently, at each occurrence, selected from the group consisting of H, C₁-C₆ alkyl and —(CH₂)phenyl;

R¹⁴ is independently, at each occurrence, selected from the group consisting of H, C₁-C₆ alkyl, halo-C₁-C₄-alkyl, C₁-C₄-alkoxycarbonylamino, (C₆-C₁₀ arylcarbonylamino), (a 5- to 10-membered heteroarylcarbonylamino) and —(CH₂)ₙ¹ phenyl substituted by 0 to 3 groups independently selected from the group consisting of halo, C₁-C₄ alkoxy, C₁-C₄ alkyl, cyclopropyl, CF₃, OCF₃, 5- or 6-membered heteroaryl, OH, OCHF₂, di-C₁-C₄-alkylamino, and cyano,

R⁷ is selected from the group consisting of H, halo, hydroxyl, oxo, C₁-C₄ alkyl, hydroxy-C₁-C₄-alkyl, C₁-C₄-alkoxy-C₁-C₄-alkyl, halo-C₁-C₄-alkyl, which contains 1 to 5 halogens, C₁-C₄-alkoxy, and halo-C₁-C₄-alkoxy;

or R⁶ and R⁷ can be taken together with the carbons to which they attach to form a C₆-C₁₀ aryl ring;

R¹⁰ is selected from the group consisting of C₁-C₄ alkyl, halo, C₁-C₄ alkoxy, and halo-C₁-C₂-alkyl, which contains 1 to 5 halogens, where halo is F or Cl;

n¹, at each occurrence, is selected from 0, 1, 2, 3, 4 or 5; and

p, at each occurrence, is selected from 0, 1 and 2.

In another embodiment, PAR4 antagonist that can be useful in the present invention are compounds, stereoisomers, tautomers, salts, solvates or prodrugs thereof, of Formula VII:

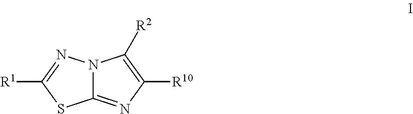

### (Formula VII)

wherein:

R¹⁰ is

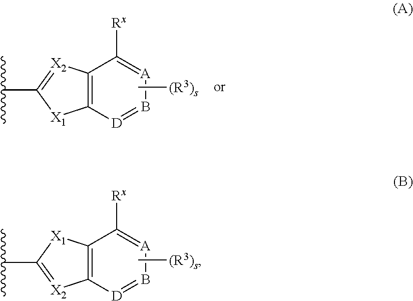

wherein A, B and D are the same or different and are independently selected from N and C, provided that A, B and D represent at least 1 carbon atom and at most 2 N atoms;

X₁ is selected from O, S or NR⁴;

X₂ is selected from CH, CR⁵ or N;

R¹ is selected from the group consisting of:
halo,
C₁-C₄ alkyl,
C₂-C₃ alkenyl,
C₂-C₃ alkynyl,
C₁-C₄ alkoxy,
C₁-C₄ alkylthio,
phenylthio,
C₁-C₄ alkylNH,
C₁-C₄-alkylOC₁-C₄-alkyl,
(C₁-C₄ alkyl)₂N—,
C₃-C₆ cycloalkyl,
4- to 10-membered heterocyclyl,
halo-C₁-C₂-alkyl, which contains 1 to 5 halogens, where halo is F or Cl,
halo-C₁-C₂-alkoxy, which contains 1 to 5 halogens, where halo is F or Cl,
C₁-C₄-alkoxycarbonyl-C₁-C₄-alkylthio, and
C₁-C₄-alkoxycarbonyl-C₁-C₄-alkoxy;

R² is selected from the group consisting of:
H,
halo,
C₁-C₄ alkyl,
C₁-C₄ alkoxy, and
cyano;

R^{x}, at each occurrence, is independently selected from the group consisting of:
H,
halo which is F, Cl, Br or I,
NR⁶R⁷,
NO₂,
cyano,
OH,
C₁-C₄ alkoxy substituted with 0 to 3 R^{a1} groups,
C₁-C₄ alkylthio substituted with 0 to 3 R^{a1} groups,
carboxy,
carbonyl,
C₁-C₄ alkoxycarbonyl substituted with 0 to 3 R^{a1} groups,
C₁-C₄ alkylcarbonyl substituted with 0 to 3 R^{a1} groups,
C(═O)NR⁶R⁷,
C₁-C₄ alkylsulfonyl substituted with 0 to 3 R^{a1} groups,
S(═O)₂NR⁶R⁷,
C₁-C₄ alkyl substituted with 0 to 3 R^{a1} groups,
fluoro-C₁-C₄-alkyl, which contains 1 to 5 fluorines, or
fluoro-C₁-C₄-alkoxy, which contains 1 to 5 fluorines; or

R^{x} is selected from Y—Z—, where:

Z is a linker which is selected from the group consisting of:
a single bond,
—O—,
—S—,

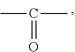

—NH—,
C₁-C₄ alkyl which is independently substituted with 0 to 3 R^{a1} groups;
C₁-C₄ alkyloxy wherein the alkyl portion is independently substituted with 0 to 3 R^{a1} groups;
C₁-C₄ alkylthio wherein the alkyl portion is independently substituted with 0 to 3 R^{a1} groups;
C₁-C₄ alkyloxy-C₁-C₄-alkyl wherein any alkyl portion is independently substituted with 0 to 3 R^{a1} groups;
C₁-C₄-alkylthio-C₁-C₄-alkyl wherein any alkyl portion is independently substituted with 0 to 3 R^{a1} groups;
—S—C₁-C₄-alkyl wherein the alkyl portion is independently substituted with 0 to 3 R^{a1} groups;
—O—C₁-C₄-alkyl wherein the alkyl portion is independently substituted with 0 to 3 R^{a1} groups; and
C₂-C₆-alkynyl which is substituted with 0 to 3 R^{a1} groups; and

Y is selected from the group consisting of:
C₁-C₄-alkyloxy-C₁-C₄-alkyl(C₁-C₄-alkyl),
C₆-C₁₀ aryl substituted by 0 to 3 R^{a5} groups,
6- to 10-membered heteroaryl substituted by 0 to 3 R^{a5} groups,
4- to 10-membered heterocyclyl substituted by 0 to 3 R^{a5} groups or 0 to 1 R^{b5} groups, and
C₃-C₁₀ cycloalkyl substituted by 0 to 3 R^{a5} groups;

R³, at each occurrence, is R^{3a}, R^{3b} or R^{3d}, each of which is independently selected from the group consisting of:
H,
halo,
NR⁶R⁷,
NO₂,
cyano,
CF₃,
OH,
C₂-C₄ alkynyl substituted with 0 to 2 R^{a1} groups,
C₁-C₄ alkoxy substituted with 0 to 2 R^{a1} groups,
C₁-C₄ alkylthio substituted with 0 to 2 R^{a1} groups,
carboxy,
—OCH═O,
C₁-C₄ alkoxycarbonyl substituted with 0 to 2 R^{a1} groups,
C₁-C₄ alkylcarbonyl substituted with 0 to 2 R^{a1} groups,
C(═O)NR⁶R⁷,
C₁-C₄ alkylsulfonyl substituted with 0 to 2 R^{a1} groups,
S(═O)₂NR⁶R⁷,
NR⁶C(═O)R⁷,
C₁-C₄ alkyl substituted with 0 to 2 R^{a1} groups,
fluoro-C₁-C₄-alkyl, which contains 1 to 5 fluorines,
fluoro-C₁-C₄-alkoxy, which contains 1 to 5 fluorines,
phenyl, where phenyl is substituted with 0 to 2 R^{a5} groups,
phenyloxy, where phenyl is substituted with 0 to 2 R^{a5} groups,
phenyl-C₁-C₄-alkoxy, where phenyl is substituted with 0 to 2 R^{a5} groups,
5- to 10-membered heteroaryl-C₁-C₄-alkoxy, where heteroaryl is substituted with 0 to 2 R^{a5} groups, and
4- to 10-membered heterocyclo-C₁-C₄-alkoxy, where heterocyclo is substituted with 0 to 2 R^{a5} groups;

R⁴ is independently selected from the group consisting of H and C₁-C₄ alkyl;

R⁵ is independently selected from the group consisting of H, halo and C₁-C₄ alkyl;

R⁶ and R⁷ are, at each occurrence, independently selected from the group consisting of:
H,
C₁-C₄ alkyl substituted with 0 to 2 R^{a1} groups,
C₁-C₄-alkoxy-C₁-C₄-alkyl, or
—(CH₂)ₙ-phenyl,
   alternatively, R⁶ and R⁷, when attached to the same nitrogen, combine to form a 4- to 6-membered heterocyclic ring containing carbon atoms and 1 to 2 additional heteroatoms selected from N, NR^{c}, O, and S(O)ₚ;

R^{a1} is, at each occurrence, independently selected from the group consisting of:
H,
═O,
halo,
OCF₃,
CF₃,
OCHF₂,
C₁-C₄ alkyl substituted with 1 to 5 fluorines,
C₁-C₄ alkyl,
C₁-C₄ alkoxy,
C₁-C₄ alkylthio,
C₃-C₆ cycloalkyl,
C₃-C₆ cycloalkyloxy,
phenyl substituted by 0 to 3 R^{a5a} groups independently selected from the group consisting of halo, C₁-C₃ alkoxy, C₁-C₃ alkyl, CF₃, OCF₃, OCHF₂, and cyano,
OH,
CN,
NO₂,
NR^{6a}R^{7a},
carboxy,
C₁-C₄ alkoxycarbonyl,
C(═O)NR^{6a}R^{7a},
C₁-C₄ alkylsulfonyl, and
S(═O)₂NR^{6a}R^{7a};

R^{a5} is, at each occurrence, independently selected from the group consisting of:
H,
halo,
OCF₃,
CF₃,
OCHF₂,
C₁-C₆ alkyl independently substituted with 1 to 5 fluorines, hydroxyl, C₁-C₄ alkoxy, C₃-C₆ cycloalkyl, or amino,
C₁-C₄ alkyl,
C₁-C₄ alkoxy,
C₁-C₄ alkylthio,
C₃-C₆ cycloalkyloxy,
OH,
CN,
NO₂,
NR^{8a}R^{9a},
carboxy,
C₁-C₄ alkoxycarbonyl,
C(═O)NR^{6a}R^{7a},
C₆-C₁₀-arylcarbonylamino-C₁-C₄-alkyl(phenyl)carbonyl,
5- to 10-membered heteroarylcarbonylamino-C₁-C₄-alkyl(phenyl)carbonyl,
C₆-C₁₀ arylcarbonyl substituted with 0 to 5 R^{a5a} groups independently selected from the group consisting of halo, C₁-C₄ alkoxy, halo-C₁-C₄ alkoxy, C₁-C₄ alkyl, halo-C₁-C₄ alkyl, C₃-C₆ cycloalkyl, cyano, nitro, NR^{6a}R^{7a}, OH, C₁-C₄-alkylcarbonyloxy-C₁-C₄-alkyl, hydroxy-C₁-C₄-alkyl, COOR⁸a, SO₂R^{8a}, (C═O)NR^{6a}R^{7a}, SO₂NR^{6a}R^{7a}, N(R^{8a})(C═O)NR^{6a}R^{7a}, N(R^{8a})(C═O)OR^{8a}, N(R^{8a})(C═O)R^{8a}, NR^{8a}S(O)R^{8a}, NR^{8a}SO₂R^{8a}, O(C═O)NR^{6a}R^{7a}, O(C═O)OR^{8a}, O(C═O)R^{8a}, (C═O)OR^{8a}, and 5-6-membered heteroaryl,
C₁-C₄-alkyloxycarbonylamino-C₁-C₄-alkyl(phenyl)carbonyl,
C₁-C₆ alkylsulfonyl,
S(═O)₂NR^{6a}R^{7a},
phenyloxy, wherein the phenyl is substituted by 0 to 5 R^{a5a} groups independently selected from the group consisting of halo, C₁-C₄ alkoxy, halo-C₁-C₄ alkoxy, C₁-C₄ alkyl, halo-C₁-C₄ alkyl, C₃-C₆ cycloalkyl, cyano, nitro, NR^{6a}R^{7a}, OH, C₁-C₄-alkylcarbonyloxy-C₁-C₄-alkyl, hydroxy-C₁-C₄-alkyl, COOR^{8a}, SO₂R^{8a}, (C═O)NR^{6a}R^{7a}, SO₂NR^{6a}R^{7a}, N(R^{8a})(C═O)NR^{6a}R^{7a}, N(R8a)(C═O)OR^{8a}, N(R^{8a})(C═O)R^{8a}, NR^{8a}S(O)R^{8a}, NR^{8a}SO₂R^{8a}, O(C═O)NR^{6a}R^{7a}, O(C═O)OR^{8a}, O(C═O)R^{8a}, (C═O)OR^{8a}, and 5-6-membered heteroaryl,
phenylthio, wherein the phenyl is substituted by 0 to 5 R^{a5a} groups independently selected from the group consisting of halo, C₁-C₄ alkoxy, halo-C₁-C₄ alkoxy, C₁-C₄ alkyl, halo-C₁-C₄ alkyl, C₃-C₆ cycloalkyl, cyano, nitro, NR^{6a}R^{7a}, OH, C₁-C₄-alkylcarbonyloxy-C₁-C₄-alkyl, hydroxy-C₁-C₄-alkyl, COOR^{8a}, SO₂R^{8a}, (C═O)NR^{6a}R^{7a}, SO₂NR^{6a}R^{7a}, N(R^{8a})(C═O)NR^{6a}R^{7a}, N(R^{8a})(C═O)OR^{8a}, N(R^{8a})(C═O)R^{8a}, NR^{8a}(O)R^{8a}, NR^{8a}SO₂R^{8a}, O(C═O)NR^{6a}R^{7a}, O(C═O)OR^{8a}, O(C═O)R^{8a}, (C═O)OR^{8a}, and 5-6-membered heteroaryl,
C₆-C₁₀-aryl-C₁-C₄-alkoxy, wherein the aryl is substituted by 0 to 5 R^{a5a} groups independently selected from the group consisting of halo, C₁-C₄ alkoxy, halo-C₁-C₄ alkoxy, C₁-C₄ alkyl, halo-C₁-C₄ alkyl, C₃-C₆ cycloalkyl, cyano, nitro, NR^{6a}R^{7a}, OH, C₁-C₄-alkylcarbonyloxy-C₁-C₄-alkyl, phenyl, phenyloxy, benzyloxy, hydroxy-C₁-C₄-alkyl, COOR^{8a}, SO₂R^{8a}, (C═O)NR^{6a}R^{7a}, SO₂NR^{6a}R^{7a}, N(R^{8a})(C═O)NR^{6a}R^{7a}, N(R^{8a})(C═O)OR^{8a}, N(R^{8a})(C═O)R^{8a}, NR^{8a}S(O)R^{8a}, NR^{8a}SO₂R^{8a}, O(C═O)NR^{6a}R^{7a}, O(C═O)OR^{8a}, O(C═O)R^{8a}, (C═O)OR^{8a}, and 5-6-membered heteroaryl,
5- to 10-membered heteroaryl-C₁-C₃-alkoxy, wherein the heteroaryl is substituted by 0 to 5 R^{a5a} groups independently selected from the group consisting of halo, C₁-C₄ alkoxy, halo-C₁-C₄ alkoxy, C₁-C₄ alkyl, halo-C₁-C₄ alkyl, C₃-C₆ cycloalkyl, cyano, nitro, NR^{6a}R^{7a}, OH, C₁-C₄-alkylcarbonyloxy-C₁-C₄-alkyl, phenyl, phenyloxy, benzyloxy, hydroxy-C₁-C₄-alkyl, COOR^{8a}, SO₂R^{8a}, (C═O)NR^{6a}R^{7a}, SO₂NR^{6a}R^{7a}, N(R^{8a})(C═O)NR^{6a}R^{7a}, N(R^{8a})(C═O)OR^{8a}, N(R^{8a})(C═O)R^{8a}, NR^{8a}(O)R^{8a}, NR^{8a}SO₂R^{8a}, O(C═O)NR^{6a}R^{7a}, O(C═O)OR^{8a}, O(C═O)R^{8a}, (C═O)OR^{8a}, and 5-6-membered heteroaryl, and
phenyl-C₁-C₃-alkyl, wherein the phenyl is substituted by 0 to 5 R^{a5a} groups independently selected from the group consisting of halo, C₁-C₄ alkoxy, halo-C₁-C₄ alkoxy, C₁-C₄ alkyl, halo-C₁-C₄ alkyl, C₃-C₆ cycloalkyl, cyano, nitro, NR^{6a}R^{7a}, OH, C₁-C₄-alkylcarbonyloxy-C₁-C₄-alkyl, hydroxy-C₁-C₄-alkyl, COOR^{8a}, SO₂R^{8a}, (C═O)NR^{6a}R^{7a}, SO₂NR^{6a}R^{7a}, N(R^{8a})(C═O)NR^{6a}R^{7a}, N(R^{8a})(C═O)OR^{8a}, N(R^{8a})(C═O)R^{8a}, NR^{8a}S(O)R^{8a}, NR^{8a}SO₂R^{8a}, O(C═O)NR^{6a}R^{7a}, O(C═O)OR^{8a}, O(C═O)R^{8a}, (C═O)OR^{8a}, and 5-6-membered heteroaryl;

R^{b5} is, at each instance, independently selected from the group consisting of:
C₆-C₁₀ aryl substituted by 0 to 3 R^{a1} groups, and
6- to 10-membered heteroaryl substituted by 0 to 3 R^{a1} groups,

R^{6a} and R^{7a} are, at each occurrence, independently selected from the group consisting of:
H,
C₁-C₆ alkyl, independently substituted with 1 to 5 fluorines, hydroxyl, C₁-C₄ alkoxy, C₃-C₆ cycloalkyl, or amino, and
—(CH₂)ₙ-phenyl independently substituted with 1 to 3 fluorines, hydroxyl, C₁-C₄ alkoxy, fluoro-C₁-C₂ alkoxy, C₃-C₆ cycloalkyl, or amino,
   alternatively, R^{6a} and R^{7a}, when attached to the same nitrogen, combine to form a 4- to 6-membered heterocyclic ring containing carbon atoms substituted by 0 to 3 groups independently selected from the group consisting of halo, CF₃, CHF₂, OCF₃, OCHF₂, OCH₂F, 5- or 6-membered heteroaryl, OH, oxo, hydroxy-C₁-C₄-alkyl, C₁-C₄ alkyl and C₁-C₄ alkoxy, and 0 to 2 additional heteroatoms selected from N, NR¹³, 0 and S(O)ₚ;

R^{8a} and R^{9a} are, at each occurrence, independently selected from the group consisting of:
H,
C₁-C₆ alkyl independently substituted with 1 to 5 fluorines, hydroxyl, C₁-C₄ alkoxy, C₃-C₆ cycloalkyl, or amino, and
—(CH₂)ₙ-phenyl independently substituted with 1 to 3 fluorines, hydroxyl, C₁-C₄ alkoxy, fluoro-C₁-C₂ alkoxy, C₃-C₆ cycloalkyl, or amino;

R^{c} is independently, at each occurrence, selected from the group consisting of H, C₁-C₆ alkyl, and —(CH₂)ₙ-phenyl;

n, at each occurrence, is selected from 0, 1, 2, 3 and 4;

p, at each occurrence, is selected from 0, 1 and 2; and

s, at each occurrence, is selected from 0, 1, 2 and 3,

provided that when R¹ is Br, R¹⁰ is other than unsubstituted

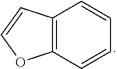

In still yet another embodiment, PAR4 antagonist that can be useful in the present invention are compounds, stereoisomers, tautomers, salts, solvates or prodrugs thereof, selected from:

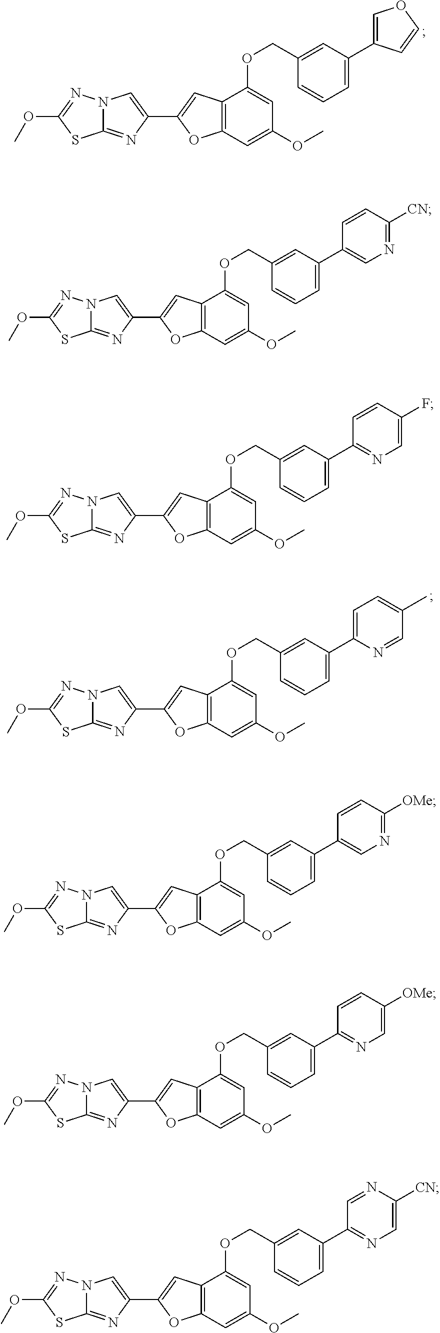

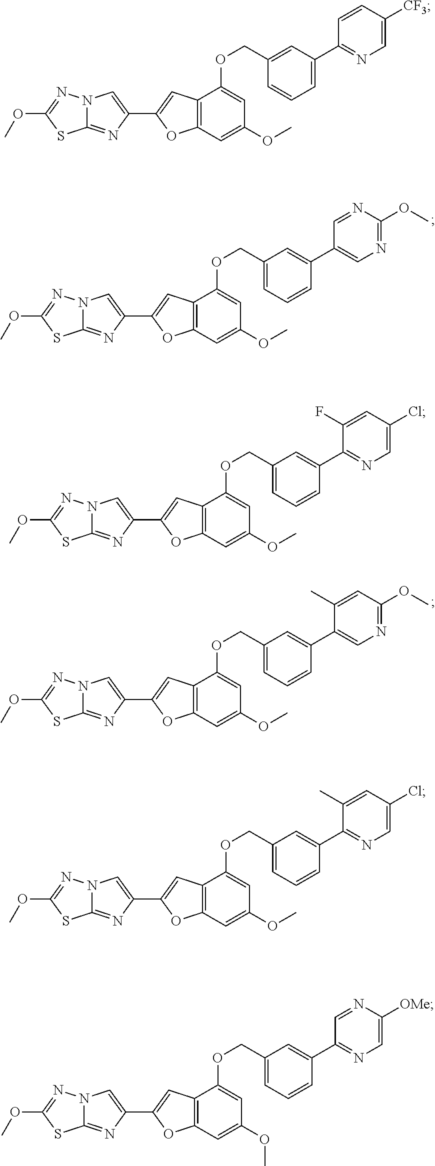

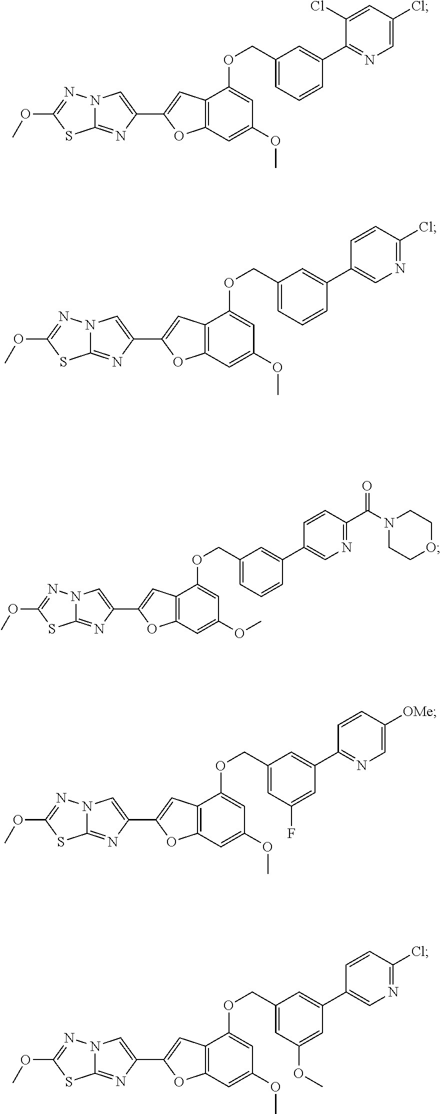 [SMILES: COc1cc(OCc2cccc(-c3ncc(Cl)cc3Cl)c2)c2cc(-c3cn4nc(OC)sc4n3)oc2c1 ; COc1cc(OCc2cccc(-c3ccc(Cl)nc3)c2)c2cc(-c3cn4nc(OC)sc4n3)oc2c1 ; COc1cc(OCc2cccc(-c3ccc(C(=O)N4CCOCC4)nc3)c2)c2cc(-c3cn4nc(OC)sc4n3)oc2c1 ; COc1ccc(-c2cc(F)cc(COc3cc(OC)cc4oc(-c5cn6nc(OC)sc6n5)cc34)c2)nc1 ; COc1cc(COc2cc(OC)cc3oc(-c4cn5nc(OC)sc5n4)cc23)cc(-c2ccc(Cl)nc2)c1]

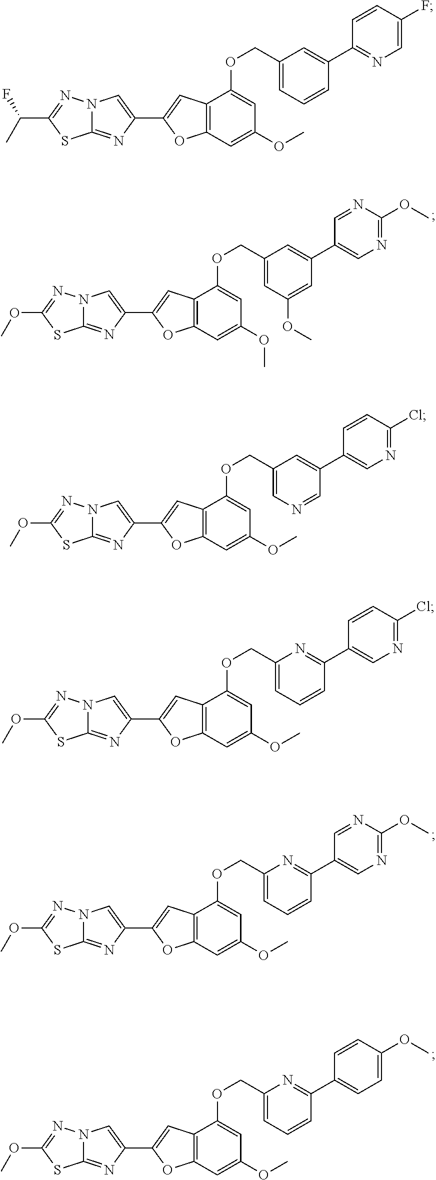

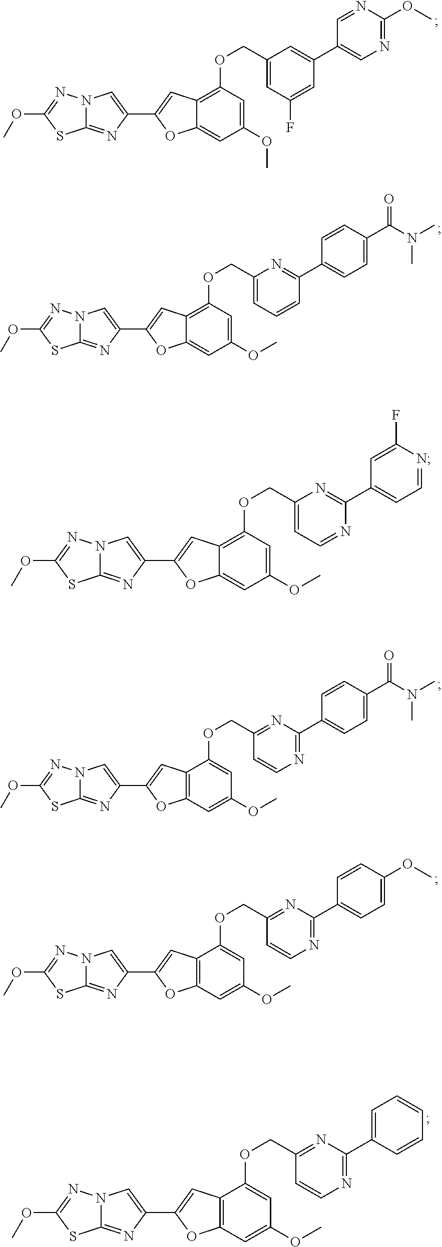

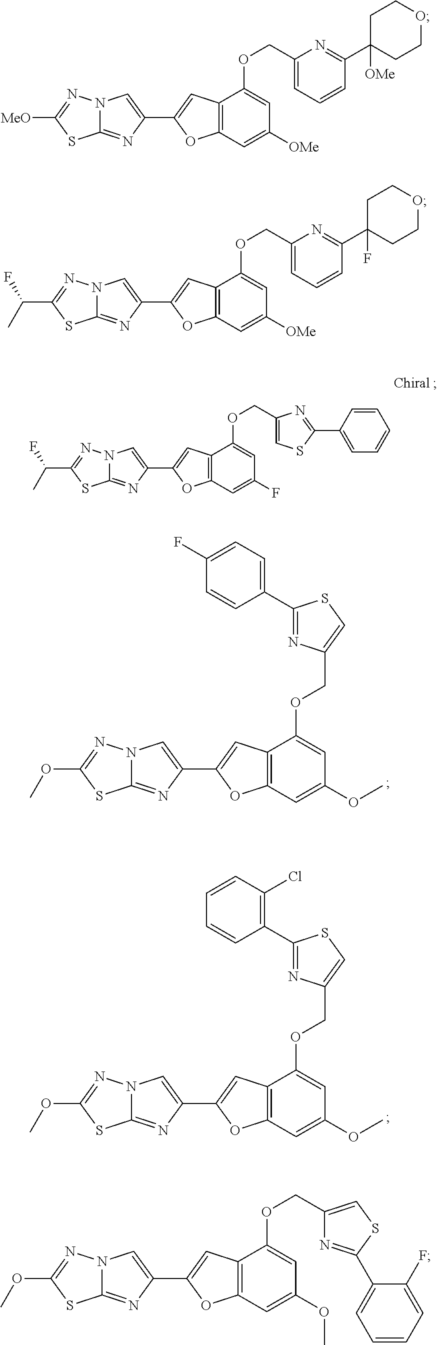

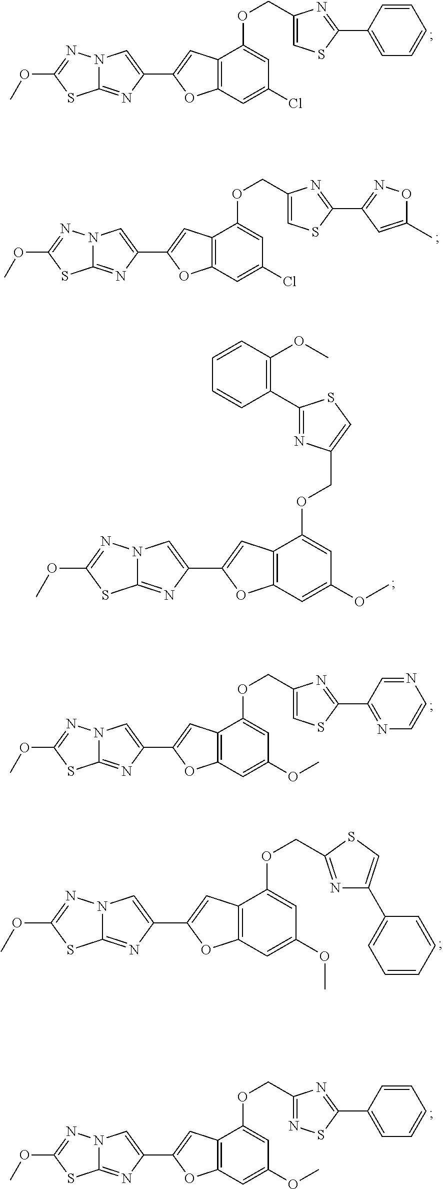

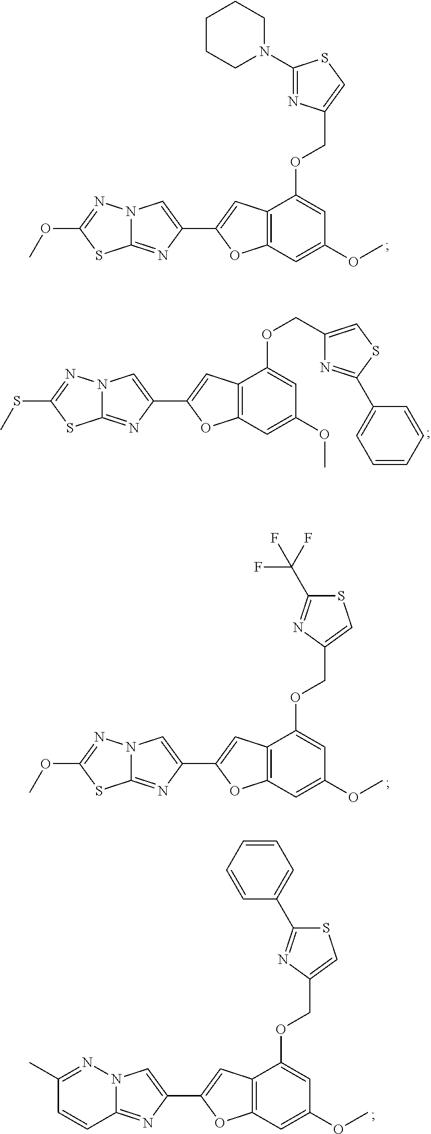

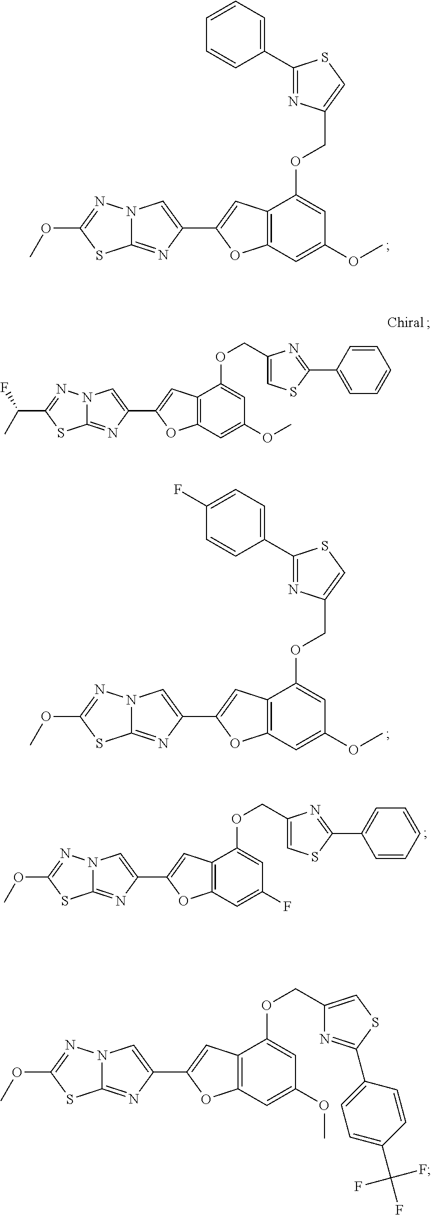

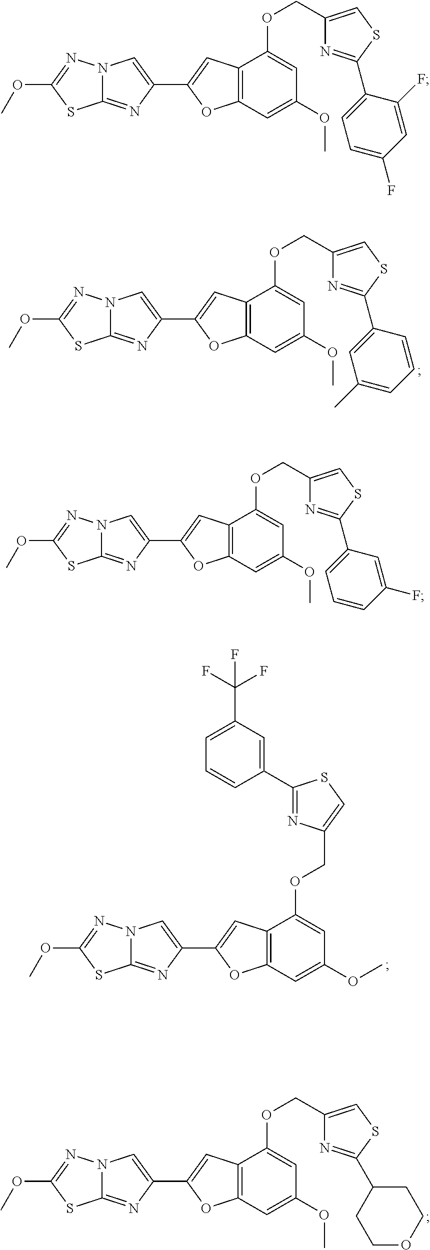

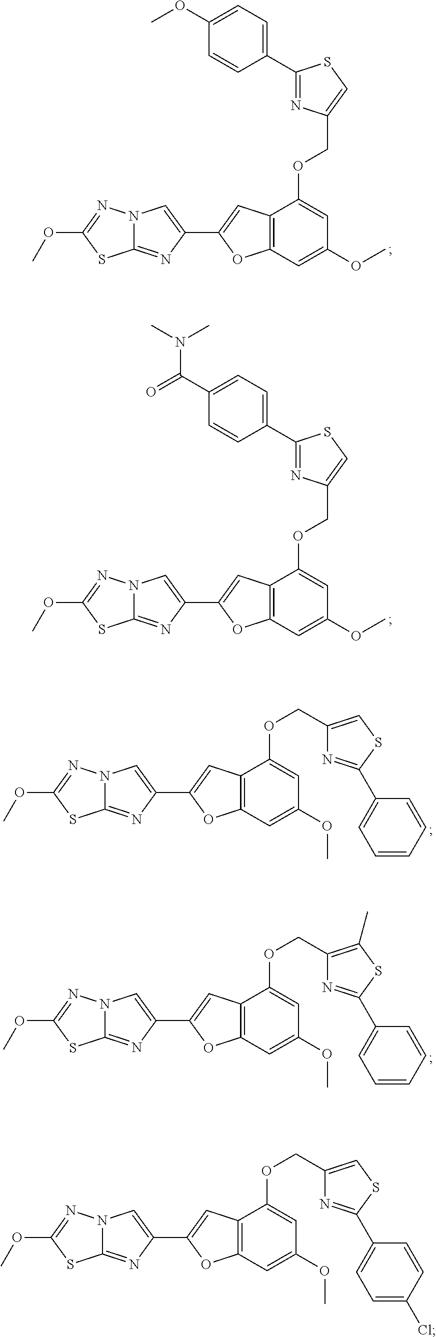

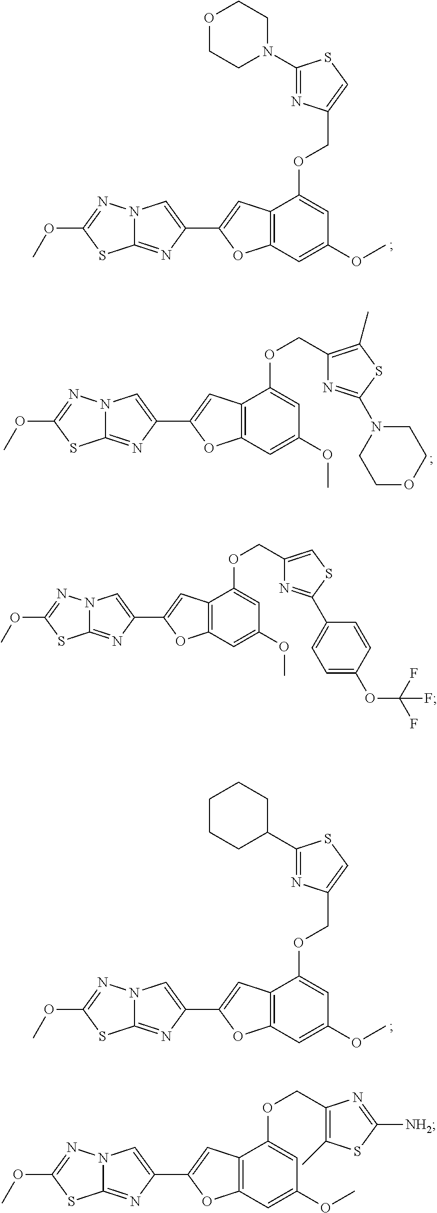

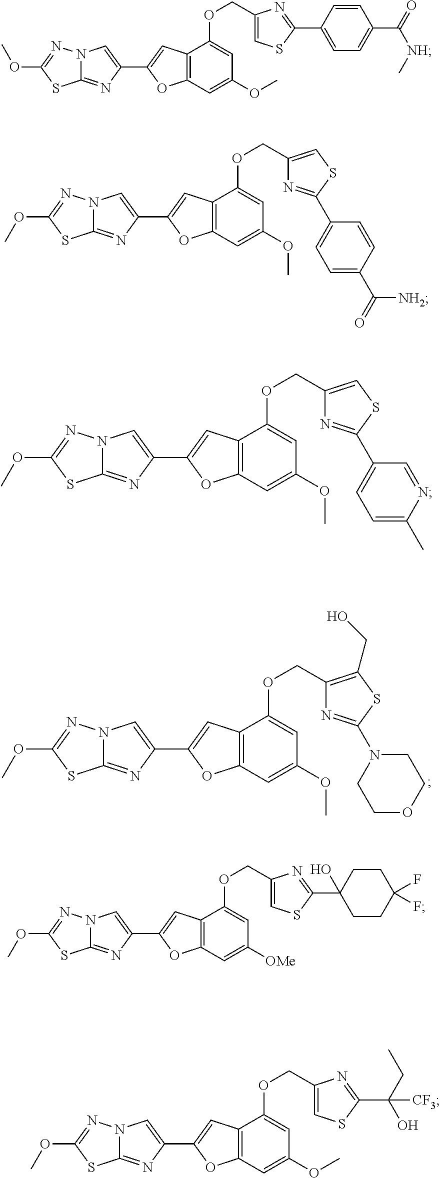

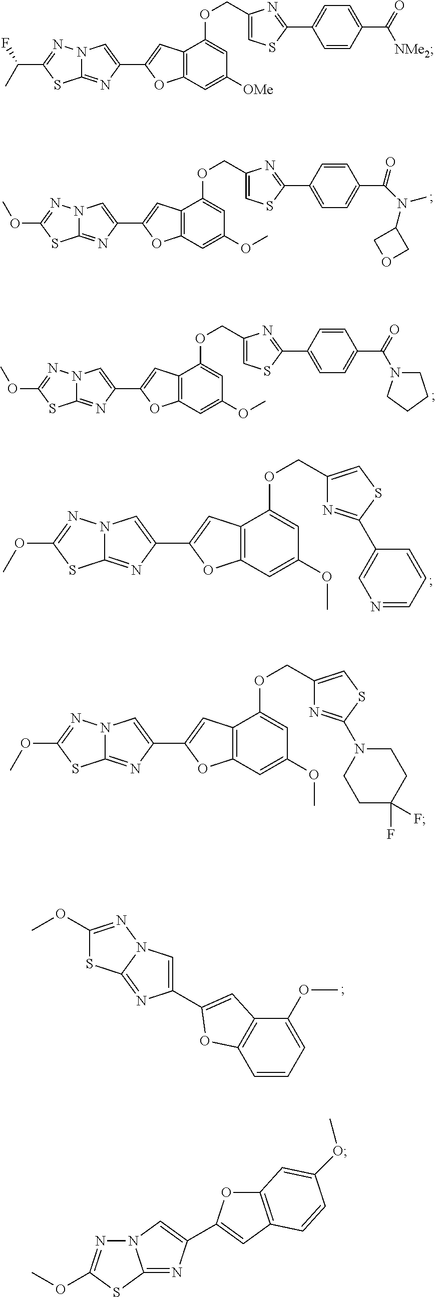

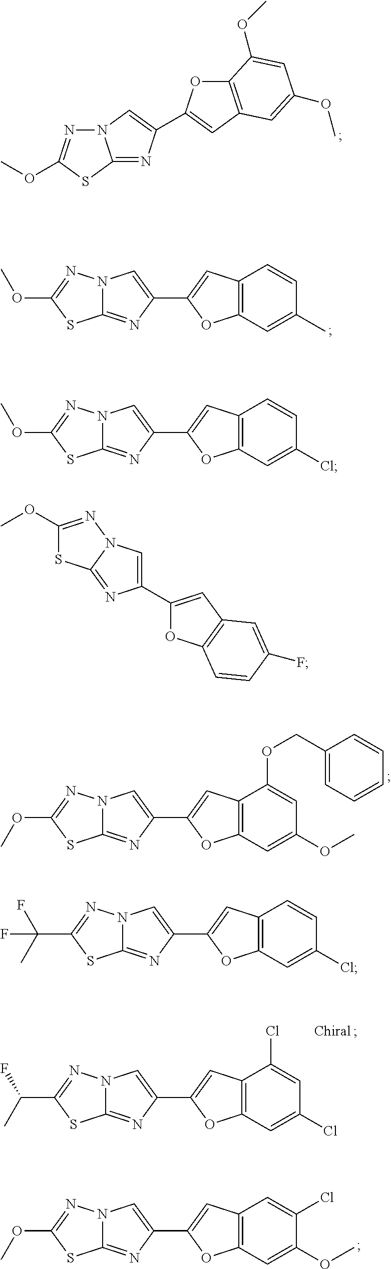

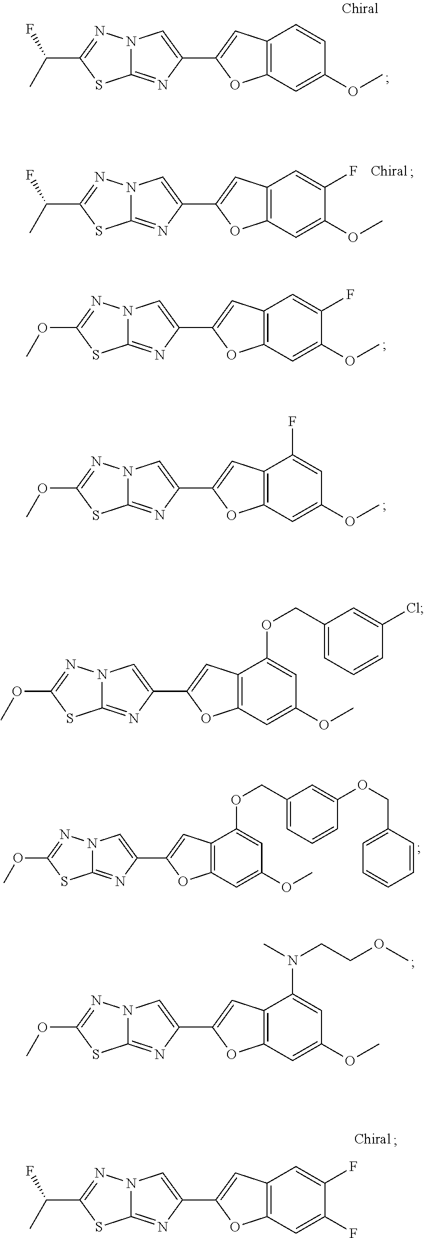

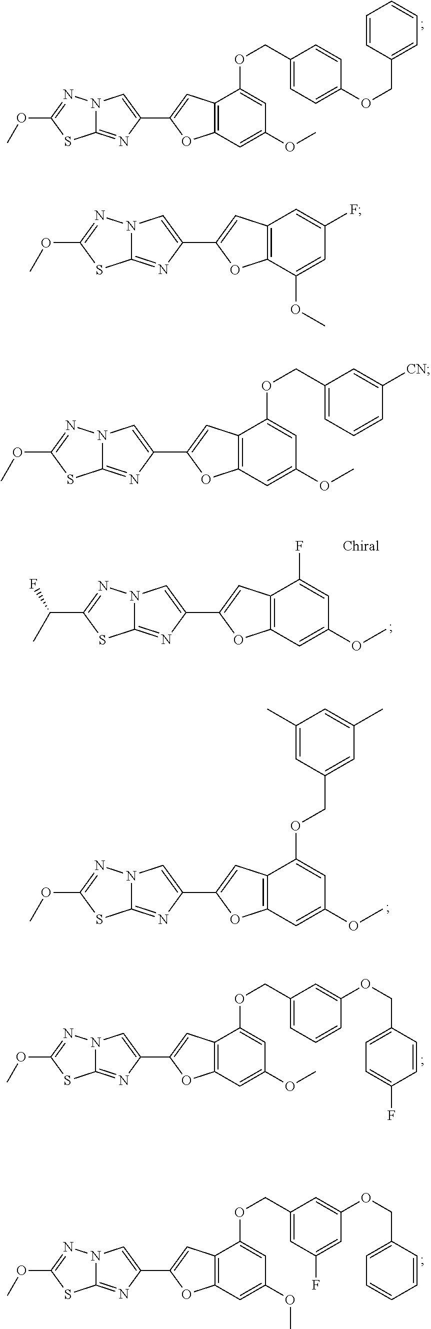

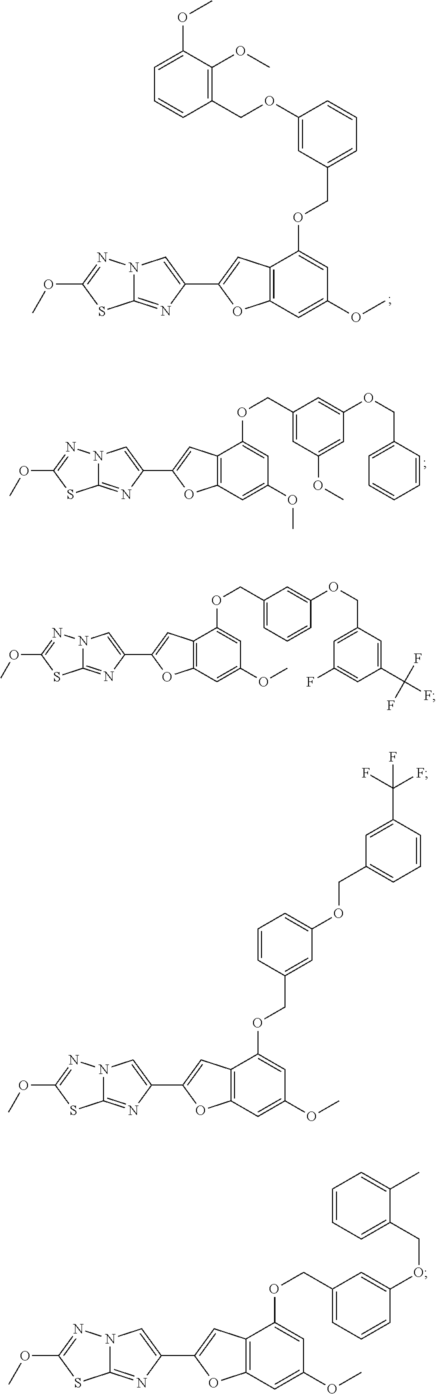

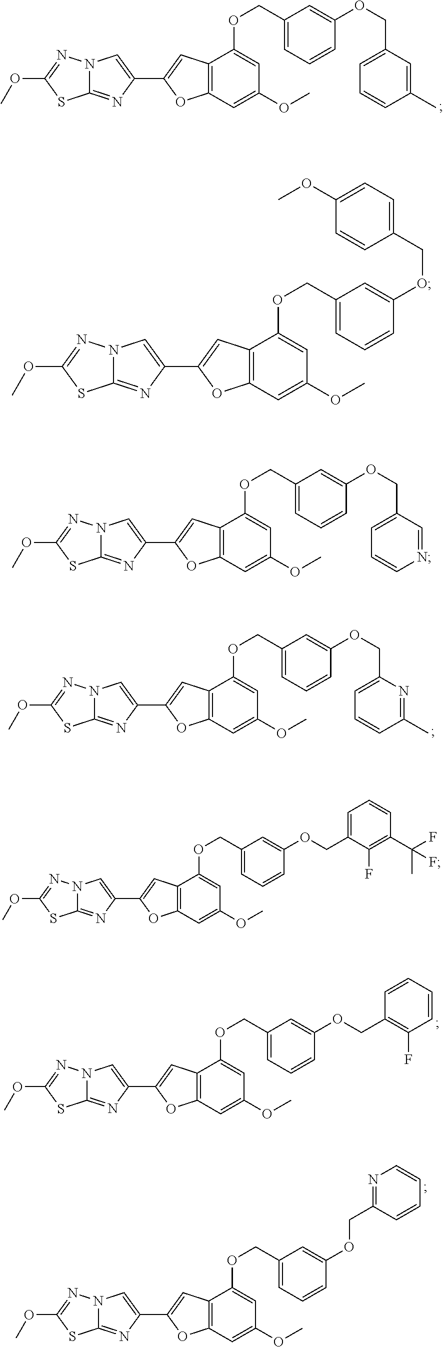

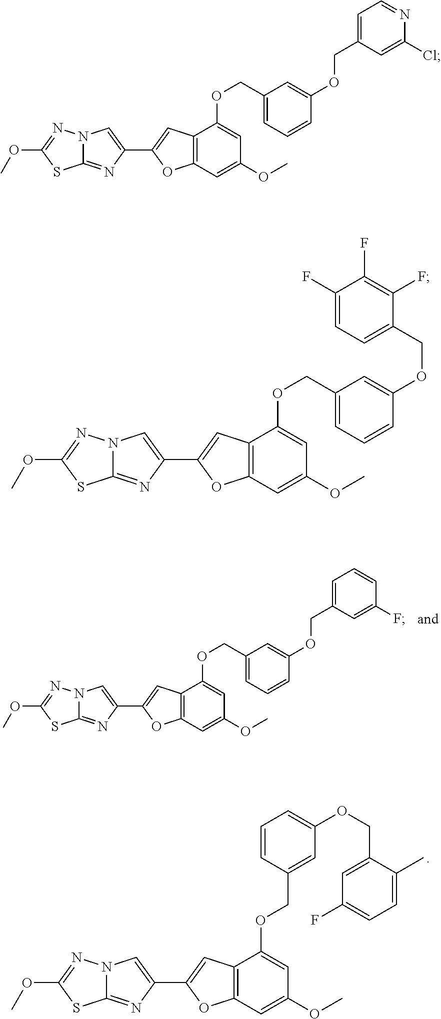

In other embodiments, the PAR4 antagonist useful for the invention also includes PAR4 antagonists known in the art. There are several early reports of preclinical studies of PAR4 inhibitors. Lee, F-Y. et al., “Synthesis of 1-Benzyl-3-(5′-hydroxymethyl-2′-furyl)indazole Analogues as Novel Antiplatelet Agents”, J. Med. Chem., 44(22):3746-3749 (2001) discloses in the abstract that the compound

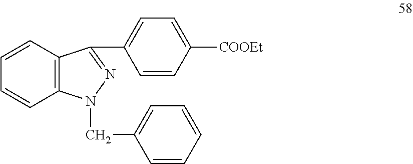 [SMILES: CCOC(=O)c1ccc(-c2nn(Cc3ccccc3)c3ccccc23)cc1]

“was found to be a selective and potent inhibitor or protease-activated receptor type 4 (PAR4)-dependent platelet activation.” EP1166785 A1 and EP0667345, which are incorporated by reference in their entireties, disclose various pyrazole derivatives which are useful as inhibitors of platelet aggregation that can be used for the invention.

In some embodiments, the PAR4 antagonist for the invention includes at least one PAR4 antagonist compound disclosed herein or a stereoisomer, tautomer, pharmaceutically acceptable salt, solvate, or prodrug ester thereof.

In some embodiments, the PAR4 antagonist for the invention is formulated as a pharmaceutical composition, which includes a pharmaceutically acceptable carrier and a therapeutically effective amount of a compound of Formula V, or stereoisomers, tautomers, pharmaceutically acceptable salts, prodrug esters, or solvates thereof, alone or in combination with another therapeutic agent.

In some embodiments, the pharmaceutical composition for the invention further includes another therapeutic agent(s). In certain embodiments, the additional therapeutic agent(s) are an anti-platelet agent or a combination thereof. In other embodiments, the anti-platelet agent(s) are P2Y12 antagonists and/or aspirin. In yet other embodiments, the P2Y12 antagonists are clopidogrel, ticagrelor, or prasugrel. In still other embodiments, the additional therapeutic agent(s) are an antithrombotics or a combination thereof. In certain embodiments, the anticoagulant agent(s) are FXa inhibitors or thrombin inhibitors. In other embodiments, the FXa inhibitors are apixaban or rivaroxaban. In some embodiments, the thrombin inhibitor is dabigatran.

### V. METHODS OF DETERMINING WHETHER PAR4 ANTAGONIST DOSE SHOULD BE ADJUSTED

The present invention also includes methods for determining if the dose of PAR4 antagonist being administered to a subject is an effective dose. In certain embodiments, such methods comprise (i) obtaining a blood sample from a subject that has been treated with a PAR4 antagonist, (ii) treating platelets from the blood sample with a PAR4 agonist in vitro, (iii) measuring platelet activation and (iv) comparing the platelet activation in the blood sample following the PAR4 antagonist treatment with the platelet activation in a blood sample obtained prior to the PAR4 antagonist treatment. In certain embodiments, if the sample obtained after the PAR4 antagonist treatment has low activity for the PAR4 agonist (a high percentage of inhibition of the PAR4 agonist by the PAR4 antagonist), then the test indicates that the PAR4 antagonist treatment is working well for the subject. In certain embodiments, if the sample obtained after the PAR4 antagonist treatment has high activity for the PAR4 agonist (a low percentage of inhibition of the PAR4 agonist by the PAR4 antagonist), then the test indicates that the PAR4 antagonist treatment may not be as effective for the subject and a dose adjustment may be required.

In other embodiments, the invention includes a method for administering a PAR4 antagonist to a subject, the method comprising: (i) obtaining a blood sample from a subject that has been treated with a PAR4 antagonist, (ii) treating platelets from the blood sample with a PAR4 agonist in vitro, (iii) measuring platelet activation (iv) comparing the platelet activation in the blood sample following the PAR4 antagonist treatment with the platelet activation in a blood sample obtained prior to the PAR4 antagonist treatment and (v)(a) increasing the subject's dose of the PAR4 antagonist if the subject's sample obtained after the PAR4 antagonist treatment has high activity for the PAR4 agonist, or (v)(b) maintaining the subject's dose of the PAR4 antagonist if the subject's sample obtained after the PAR4 antagonist treatment has low activity for the PAR4 agonist.

In certain embodiments the subject sample is determined to have low activity for a PAR4 agonist if the sample has less than 25%, 20%, 15%, 10% 5%, 2% or 1% of the activity of the blood sample obtained prior to PAR4 antagonist treatment. In certain embodiments, the subject sample is determined to have high activity for a PAR4 agonist if the sample has at least 50%, 55%, 60%, 65%, 70%, 75%, 80%, 85%, 90%, 95%, 97% or 99% of the activity of the blood sample obtained prior to PAR4 antagonist treatment.

### VI. KITS

The present invention also includes a pharmaceutical kit comprising a PAR4 agonist. The kits of the invention can be used to identify a subject who is responsive to a PAR4 agonist and/or PAR4 antagonist therapy comprising a PAR4 agonist and an instructional material, which instructs a healthcare provider to mix the PAR4 agonist with platelets in a sample taken from a subject in need of PAR4 antagonist therapy and to measure the platelet activation. Non-limiting examples of the PAR4 agonist are described in Section III herein.

In some embodiments, a kit comprises (i) a PAR4 agonist, (ii) an instructional material, which instructs a healthcare provider to mix the PAR4 agonist with platelets in a sample taken from a subject in need of PAR4 antagonist therapy and to measure the platelet activation, and (iii) an agent to measure platelet activation. The agent to measure platelet activation can detect a platelet marker, for which the level differs between resting and activated platelets. In other embodiments, the agent comprises an antibody specifically binding to one or more platelet activation markers. For example, the marker can be selected from: GPIIb/IIIa, fibrinogen binding to GPIIb/IIIa or other membrane structures, GPIa/IIa (VLA-2), binding of collagen or collagen mimetics or to other membrane structures, CD62P (P-selectin), CD63, GPIb-alpha, GPIb-beta, GP IX, GPVI, GPIV, CD43, CD100, CD147, annexin V, lactadherin, or any combinations thereof. The antibody specifically binding to one or more platelet activation markers can be conjugated to fluorescein isothiocyanate (FITC) or phycoerythrin (PE). In still other embodiments, the antibody specifically binding to one or more platelet activation markers is a polyclonal antibody or a monoclonal antibody.

In a particular embodiment, a kit comprises (i) a PAR4 agonist, (ii) an instructional material, which instructs a healthcare provider to mix the PAR4 agonist with platelets in a sample taken from a subject in need of PAR4 antagonist therapy and to measure the platelet activation, and (iii) an antibody against fibrinogen. Examples of the antibodies against fibrinogen include, but are not limited to, F-0111, LS-B2573, LS-B697, LS-B5249, LS-B381, LS-B3048, LS-B7075, LS-052057, LS-C109177, LS-C150799, MCA2760, 4440-8004, NBP1-33582, NB600-926, NBP1-47442, NBP2-11515, NB120-10070, NBP1-96183, NBP1-96180, or any combinations thereof. In a specific embodiment, an antibody against fibrinogen is a rabbit polyclonal antibody against human fibrinogen, e.g., F-0111.

In certain embodiments, a kit comprises (i) a PAR4 agonist, (ii) an instructional material, which instructs a healthcare provider to mix the PAR4 agonist with platelets in a sample taken from a subject in need of PAR4 antagonist therapy and to measure the platelet activation, and (iii) an antibody against P-selectiny. Examples of the antibodies against P-selectin include, but are not limited to CLB/Thromb/6, AK4, 1E3, CTB201, P8G6, MAB2154, ab91132, LS-B3578, LS-B3656, LS-C134593, LS-C13963, 10-667-C100, 11-450-C100, 1A-450-T100, 1Y-450-T100, OASA02343, PN IM1315, or any combinations thereof.

In some embodiments, a kit comprises (i) a PAR4 agonist, (ii) an instructional material, which instructs a healthcare provider to mix the PAR4 agonist with platelets in a sample taken from a subject in need of PAR4 antagonist therapy and to measure the platelet activation, (iii) an antibody against fibrinogen, and (iv) an antibody against P-selectin. In one embodiment, the antibody against fibrinogen is selected from F-0111, LS-B2573, LS-B697, LS-B5249, LS-B381, LS-B3048, LS-B7075, LS-052057, LS-C109177, LS-C150799, MCA2760, 4440-8004, NBP1-33582, NB600-926, NBP1-47442, NBP2-11515, NB120-10070, NBP1-96183, NBP1-96180, or any combinations thereof, and the antibody against P-selectin is selected from CLB/Thromb/6, AK4, 1E3, CTB201, P8G6, MAB2154, ab91132, LS-B3578, LS-B3656, LS-C134593, LS-C13963, 10-667-C100, 11-450-C100, 1A-450-T100, 1Y-450-T100, OASA02343, PN IM1315, or any combinations thereof. In another embodiment, the antibody against fibrinogen is F-0111, and the antibody against P-selectin is CLB/Thromb/6.

In further embodiments, a kit comprises (i) a PAR4 agonist, (ii) an instructional material, which instructs a healthcare provider to mix the PAR4 agonist with platelets in a sample taken from a subject in need of PAR4 antagonist therapy and to measure the platelet activation, (iii) one or more agents to measure platelet activation (e.g., an antibody against fibrinogen and/or an antibody against P-selectin), and (iv) a PAR4 antagonist. Non-limiting examples of a PAR4 agonist and a PAR4 antagonist are described elsewhere herein in Sections III and IV, respectively.

### EXAMPLES

### Example 1

### Agonist Activity of PAR4 Agonist Peptides

### A. PAR4 Agonist Peptide Induced Platelet Aggregation Assays

The ability of the peptides comprising Formulas (I) to (IV) to induce platelet aggregation was tested in a 96-well microplate aggregation assay format. Briefly, a washed platelet suspension with varying concentrations of agonist compounds was assayed. Aggregation was initiated by the addition of a titered test agonist peptide. The plate was then placed into a 37° C. Molecular Devices SPECTRAMAX® Plus Plate Reader (Sunnyvale, Calif.). The plate was mixed for 10 seconds before the first read and 20 seconds between each read for up to 15 minutes at 405 nM. Data was collected with SOFTMAX® 4.71 software. The plate also included an untreated control sample that served as ODmax, while buffer containing no platelets was the ODmin. Platelet aggregation was determined by subtracting the ODmax from the ODmin for the 100% aggregation value. In experimental samples, the observed transmission was subtracted from the minimum value and then compared to the 100% aggregation value to determine the percentage aggregation. ED₅₀ values were determined using Excel Fit software.

### B. FLIPR Assay in PAR4-Expressing HEK293 Cells

The activity of the PAR4 agonist were tested in PAR4 expressing cells by monitoring PAR4-induced intracellular calcium mobilization using FDSS6000 (Hamamatsu Photonics, Japan) by fluo-4. Briefly, HEK293 cells expressing human PAR4 were plated 24 hrs. prior to experiment in 96 well or 384 well, Poly-D-Lysine coated, black, clear bottom plates (Greiner Bio-One, Monroe, N.C.). Cells were plated at 40,000 cells/well (96 well) or 20,000 cells/well (384 well) and incubated at 37° C. with 5% CO₂ overnight. At the time of assay, media was replaced with 1× Hank's Buffered Saline Solution (HBSS) (with 10 mM HEPES) for the 96 well or 384 well assays respectively. The cells were then incubated for 30 minutes at room temperature followed by addition of a varying concentration of agonist peptide for measurement on the FDSS.

The following Table sets forth the results obtained when several peptides were assessed using the methods described above.

TABLE 5
EC₅₀ results for FLIPR Assay and Platelet Aggregation Assay

| | EC₅₀ (μM) | ED₅₀ (μM) |
|---|---|---|
| SEQ ID NO. | FLIPR Assay | [Platelet Aggregation] |
| 1 | 61 | 13 |
| 2 | 4.8 | NT* |
| 3 | 2.3 | 0.86 |
| 4 | 3.9 | NT* |
| 5 | 4.1 | NT* |
| 6 | 2.6 | 1.1 |
| 7 | 9.5 | NT* |
| 8 | 26 | NT* |
| 9 | 410 | NT* |
| 10 | 600 | NT* |
| 11 | 159 | NT* |
| 12 | NT* | 0.97 |
| 13 | NT* | 0.67 |
| 14 | NT* | 12.3 |
| 15 | 0.45 | 0.63 |
| 16 | NT* | 37 |
| 17 | NT* | 106 |
| 18 | NT* | 5.5 |
| 19 | NT* | 1.6 |
| 20 | NT* | 4.1 |
| 21 | NT* | 2.4 |
| 22 | NT* | 1.2 |
| 23 | NT* | 0.79 |
| 24 | 0.75 | 0.69 |
| 25 | NT* | 52 |
| 26 | NT* | 1.0 |
| 27 | NT* | 1.7 |
| 28 | 1.5 | 0.76 |
| 29 | NT* | 1.0 |
| 30 | NT* | 1.7 |
| 31 | 0.72 | 0.63 |
| 32 | 0.31 | 0.61 |
| 33 | NT* | 1.2 |
| 34 | NT* | 3.0 |

*Not Tested

### Example 2

### Platelet Activation Assay by PAR4 Agonist

The aim of the study was to use assays to determine the range of platelet responses between individual subjects to a PAR4 agonist (Compound B) (SEQ ID NO: 3) and thus to stratify the subjects into three groups: high responder, normal responder, and low responder to the PAR4 agonist. The study will thus test the responsiveness of each group to a PAR4 antagonist. A PAR1 agonist (i.e., SFFLRR (SEQ ID NO: 39)) is a control and described herein as Compound A. The degree of platelet activation was assessed by incubating whole blood with the separate compounds and measuring platelet-bound fibrinogen and P-selectin (CD62P) expression, as markers representing activation of the GPIIbIIIa complex and a granule release respectively.

The subjects for the current study were recruited from the National Health Service Blood and Transplant Cambridge BioResource (www.cambridgebioresource.org.uk/) after gaining informed, written consent.

Blood was drawn from the antecubital fossa, contra lateral to the one used for routine whole blood or platelet donation. A 21-gauge butterfly needle and a Vacuette tube (Greiner bio-one, Stonehouse, Gloucestershire, UK) were used following a standardised protocol. The first 3 mL of blood were discarded and a subsequent sample was taken into 3.2% sodium citrate for platelet function analysis.

PAR1 and PAR4 agonists were supplied as freeze dried peptides and reconstituted with HEPES buffered saline [HBS, 0.14M NaCl, 5 mM KCl, 1 mM MgSO₄, 10 mM HEPES (sodium salt), pH 7.4] to 1 mM concentrations prior to storing in single use aliquots at −80° C. Immediately prior to use Compound A was thawed and diluted in HBS to 10 μM, and Compound B to 100 μM.

Aspirin, apyrase, and hirudin, were all from Sigma Chemical Co. Ltd (Poole, Dorset, UK) and were stored at −80° C. in single-use aliquots until added to the citrated blood during the activation assays.

The antibodies used for flow cytometry were: a rabbit polyclonal FITC-labeled antibody against fibrinogen (Dako Ltd, Ely, UK), a murine phycoerythrin (PE) labeled monoclonal antibody against CD62P and a relevant PE-isotype control (Bristol Institute for Transfusion Science, Bristol, UK). HBS was used for all dilutions.

Platelet activation was measured using whole blood flow cytometry. Blood was processed within 15 minutes of venesection, with 5 μL of citrated whole blood containing aspirin (100 μM) and hirudin (10 U/mL) being added to 45 μL of HBS containing apyrase (4 U/mL), either FITC labeled antibody against fibrinogen or PE labeled antibody against CD62P, and either Compound A (1 μM) or Compound B (10 μM). After a 20 minute incubation at room temperature, reactions were stopped by 100-fold dilution in formyl saline (0.2% formaldehyde in 0.9% NaCl, both from Sigma) prior to flow cytometric analysis using either an EPICS Profile XL or FC500 flow cytometer (Beckman-Coulter Ltd., High Wycombe, UK). Platelets were identified by light scatter and results were recorded as the percentage of platelets positive for the observed level of fibrinogen binding or binding of the antibody against CD62P.

Negative controls for the P-selectin antibody were set using an appropriate isotype control and for the fibrinogen antibody using samples incubated with the antibody in the presence of 10 mM EDTA.

### Example 3

### Calibration Study Using Other Agonists or Antibodies

The agonist concentration chosen for testing individuals in Example 2 was the concentration that gave the greatest variation in results between subjects, i.e., as a mid-range concentration. A low concentration would not be suitable, as most individuals would have no detectable response or very weak responses. This narrow result range would limit the ability to discriminate between individuals. Similarly, a concentration that is too high would give a maximum result and again give a narrow range of results, limiting the ability to detect differences between individuals.

The antibodies used to detect the platelet activation are standardized by using titration studies with resting and maximally against activated samples. An antibody concentration has been used that gives the maximum difference in results between the resting sample and the maximally activated sample. The maximally activated sample is generated by using a high enough concentration of agonist to give a maximum response, that is >95% of platelets are expressing the activation marker, e.g., bind fibrinogen or express P-selectin.

New batches of agonists and antibodies can be standardized by titrating in parallel with existing reagents to find the concentrations that gives the same result as the existing reagent.

The foregoing description of the specific aspects will so fully reveal the general nature of the disclosure that others can, by applying knowledge within the skill of the art, readily modify and/or adapt for various applications such specific aspects, without undue experimentation, without departing from the general concepts provided. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

### Example 4

### Categorization of PAR4 Agonist Responders

### Introduction

Within the general population, several studies have confirmed the long-held notion that the response of platelets to agonists is highly variable, whilst for an individual the level of responsiveness is remarkably consistent over time (Tofler et al., 1987, Fontana et al., 2003, Hetherington et al., 2005, Yee et al., 2005, Panzer et al., 2006, Jones et al., 2007). The recognition that platelet reactivity is at least partly genetically controlled, and therefore potentially stable over time is supported by the platelet aggregation observations in the Framingham Heart Study that found that heritable factors played a key role in aggregation responses (O'Donnell et al., 2001). Additional studies have identified genetic variants underlying platelet function and are compatible with the notion that the inter-individual variation in platelet parameters like their count, volume and function is to a large extent inherited and therefore stable (Jones et al., 2009, Soranzo et al., 2009a, Soranzo et al., 2009b, Johnson et al., 2010, Gieger et al., 2011).

Previously, flow cytometric whole blood platelet function assays have been used to demonstrate the wide but stable range of inter-individual responses to two agonists, cross-linked collagen-related peptide (CRP-XL) and adenosine 5′-diphosphate (ADP) (Jones et al., 2007).

The aim of the current study was to use the same assays for two purposes. Firstly to determine the range of platelet responses between individuals to two agonists supplied by Bristol-Myers Squibb, namely PAR1 and PAR4. The degree of platelet activation was assessed by incubating whole blood with the separate compounds and measuring platelet-bound fibrinogen and P-selectin (CD62P) expression, as markers representing activation of the glycoprotein IIbIIIa complex and a granule release respectively. This aspect of work is referred to here on in as part A.

The second part of the study, referred to as part B, relates to the potential role of platelets in atherothrombotic events such as stroke and heart attack, with platelet activation essential to the formation of embolisms and thrombi. This is demonstrated by the association between platelet hyperresponsiveness and an increased risk of mortality and morbidity in survivors of heart attack and the effectiveness of anti-platelet therapies in improving this outcome. (Trip et al., 1990, Antithrombotic Trialists, 2002, Hamm et al., 2011, Steg et al., 2013).

It has been suggested that different platelet assessments, such as hyperresponsiveness, could be indicative for determining responsiveness to antiplatelet therapies and therefore be predictive of clinical outcome. As several clinical studies have demonstrated variability in the level of platelet inhibition when patients were administrated with anti-platelet drugs (Serebruany et al., 2005), part B of the current study looks at the potential relationship between the degree of platelet responsiveness to agonists, and the efficacy of anti-platelet agents.

Recently, a new PAR4 inhibitor, UDM-2555, has been developed by Bristol-Myers Squib. This study therefore aimed to assess whether hyper-responders would require a higher concentration of the inhibitor to achieve the same level of inhibition as seen in individuals that were mid-range or hypo-responders.

Part B study was being conducted in three stages: (i) an optimal concentration-response curve of UDM-002555 for inhibition of platelet fibrinogen binding and P-selectin expression was established in 12 subjects; (ii) data analysis of the 501 subjects tested in Part A was performed to select individuals with hypo-, hyper- and mid-range responders for fibrinogen binding and P-selectin expression; (iii) the selected inhibitor concentration—response curve was tested against the identified individuals to assess the variation in inhibition responses.

### Part A

### Materials and Methods

### Subjects

The subjects for the current study were recruited from the National Health Service Blood and Transplant and Cambridge BioResource after gaining informed, written consent.

### Blood Collection

Blood was drawn from the antecubital fossa, contra lateral to the one used for routine whole blood or platelet donation. A 21-gauge butterfly needle and a Vacuette tube (Greiner bio-one, Stonehouse, Gloucestershire, UK) were used following a standardised protocol. The first 3 mL of blood were discarded and a subsequent sample was taken into 3.2% sodium citrate for platelet function analysis.

### Platelet Activation Reagents

PAR1 and PAR4 agonists were supplied as freeze dried peptides and reconstituted with HEPES buffered saline [HBS, 0.14M NaCl, 5 mM KCl, 1 mM MgSO4, 10 mM HEPES (sodium salt), pH 7.4] to 1 mM concentrations prior to storing in single use aliquots at −80° C. Immediately prior to use PAR1 was thawed and diluted in HBS to 10 μM as a working concentration to give a final assay concentration of 1 μM. Similarly PAR4 was diluted to 100 μM working concentration to give a final assay concentration of 10 μM.

The inhibitors, aspirin, apyrase, and hirudin, were all from Sigma Chemical Co. Ltd (Poole, Dorset, UK) and were stored at −80° C. in single-use aliquots until added to the citrated blood during the activation assays.

The antibodies used for flow cytometry were: rabbit FITC-anti-fibrinogen (Dako Ltd, Ely, UK), PE-anti-CD62P and a relevant PE-isotype control (Bristol Institute for Transfusion Science, Bristol, UK). HBS was used for all dilutions.

### Platelet Activation Measurements Using Whole Blood Flow Cytometry

Blood was processed within 15 minutes of venesection, with 5 μL of citrated whole blood containing aspirin (100 μM) and hirudin (10 U/mL) being added to 45 μL of HBS containing apyrase (4 U/mL), either FITC-anti-fibrinogen or PE anti-CD62P, and either PAR1 (1 μM final concentration) or PAR4 (10 μM final concentration). After a 20 minute incubation at room temperature, reactions were stopped by 100-fold dilution in formyl saline (0.2% formaldehyde in 0.9% NaCl, both from Sigma) prior to flow cytometric analysis using either an EPICS Profile XL or FC500 flow cytometer (Beckman-Coulter Ltd., High Wycombe, UK). Platelets were identified by light scatter and results were recorded as the percentage of platelets positive for the relevant activation marker.

Negative controls for the P-selectin antibody were set using an appropriate isotype control and for anti-fibrinogen using samples incubated with the antibody in the presence of 10 mM EDTA.

### Results

### Flow Cytometry Data

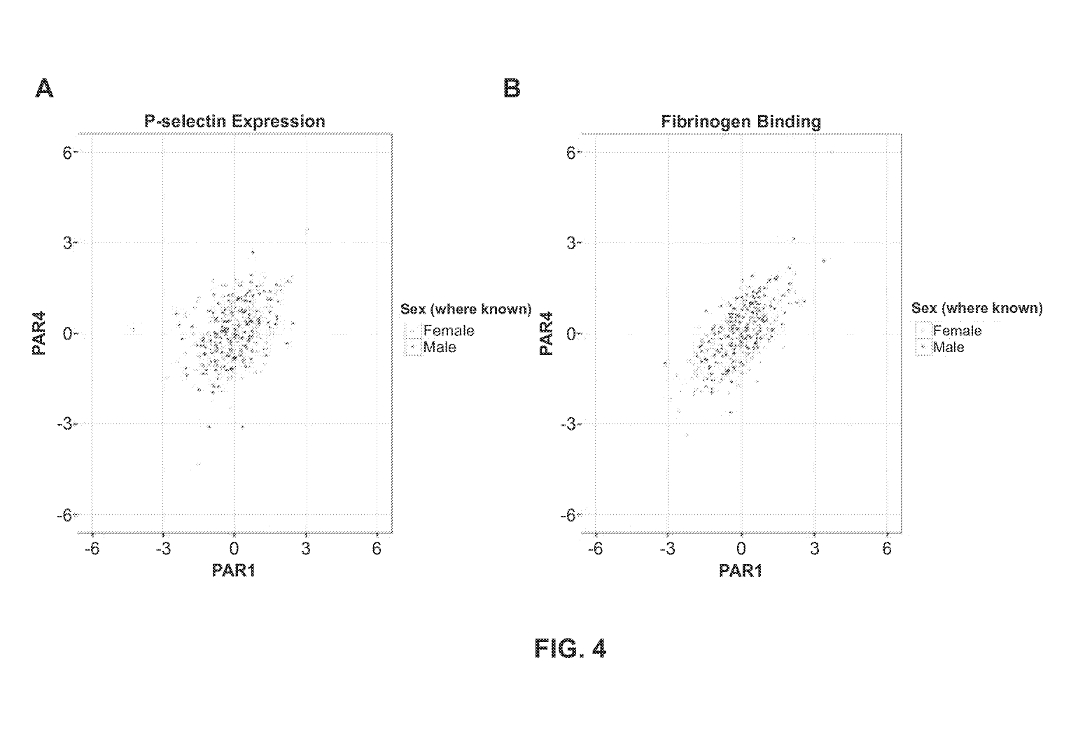

Platelet responses were measured in 501 healthy subjects (FIGS. 4A and 1B); fibrinogen binding to PAR1-stimulated platelets ranged from 1.8% to 83.6% positive [median 19.7%; interquartile range (IQR) 13.0%-29.6%], whilst fibrinogen binding in response to PAR4 ranged from 3.0% to 98.1% positive (median 32.3%; IQR 21.0%-43.4%).

The range for PAR1-induced P-selectin expression was 21.1%—98.8% positive (median 86.8%; IQR 80.0%-91.7%), and PAR4-induced P-selectin expression ranged from 29.4% to 99.0% positive (median 87.9%; IQR 82.0%-91.8%).

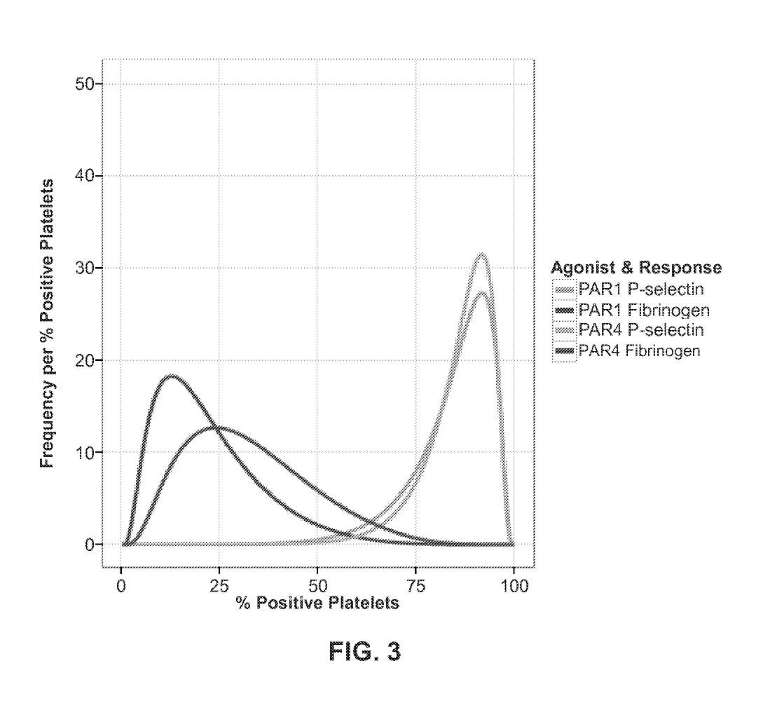

FIG. 2 summarises the observed distributions of the P-selectin and fibrinogen binding % positive platelets responses for each agonist. A logistic normal distribution was fitted to the data from each response. The logistic normal distribution is a uni-modal parametric distribution on the interval (0%-100%). The fits, which are shown in FIG. 3, allow for comparison between the each response distribution.

### Transformed Data

Data following a logistic normal distribution follow a normal distribution after logistic transformation. Therefore the data were logistic transformed the percent-positive measurements for each response. The transformation maps percentages monotonically to the number line, giving a measure of response on the logodds scale between positive and negative infinity. (0% maps to negative infinity, 50% maps to 0 and 100% maps to positive infinity.) The transformed data were then standardised, adjusting the sample mean of each logistic transformed response to zero and the sample variance of each logistic transformed response to one.

### Assessment of Study Homogeneity

To assess the homogeneity of the study over time, each of the four standardised responses was regressed on a predictor variable counting the number of days after study initiation that a sample was agonised. The inferred regression coefficients implied either that there were no systematic monotonic trends over time or that any such trend was extremely weak and could be discounted. The relationship between the standardised responses and the time of day each sample was agonised was also considered. Again, the weight of evidence supported study homogeneity.

The subjects were recruited through a variety of different sub-studies. The transformed standardised response measurements was adjusted to remove any systematic differences in mean response between the sub-studies. These might have been generated for example by slight differences in blood handling procedures.

There is no evidence of any substantial difference in response to agonism by sex, except perhaps for response to PAR4 as measured by fibrinogen binding, for which men appear to be slightly more responsive than women.

The relationships between the adjusted responses to the two agonists as measured by P-selectin expression and fibrinogen binding are shown in FIG. 4. The Pearson correlation between the adjusted responses to the two agonists is 0.42 if measured by P-selectin expression and 0.65 if measured by Fibrinogen binding. This shows that there is considerable similarity in the strength of each response to the two agonists.

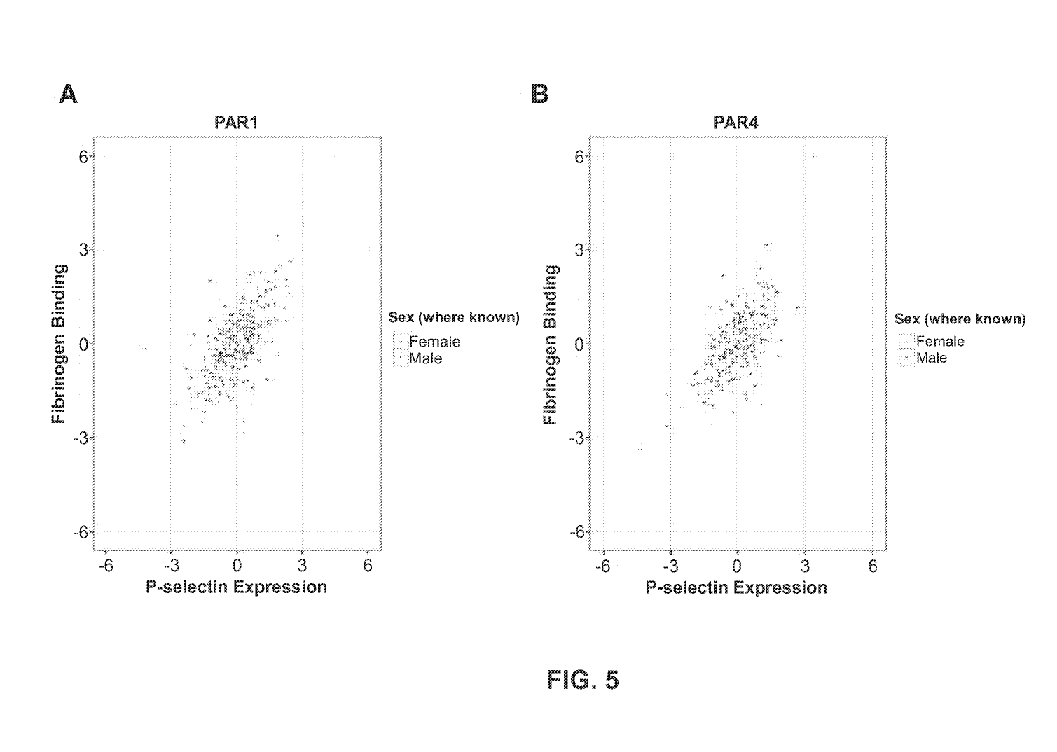

FIG. 5 shows the relationship between the two adjusted measures of response to each agonist. The Pearson correlation between the adjusted responses to PAR1 is 0.64. The Pearson correlation between the adjusted responses to PAR4 is 0.67. This shows that there is good agreement between the two response measurements for each of the two agonists.

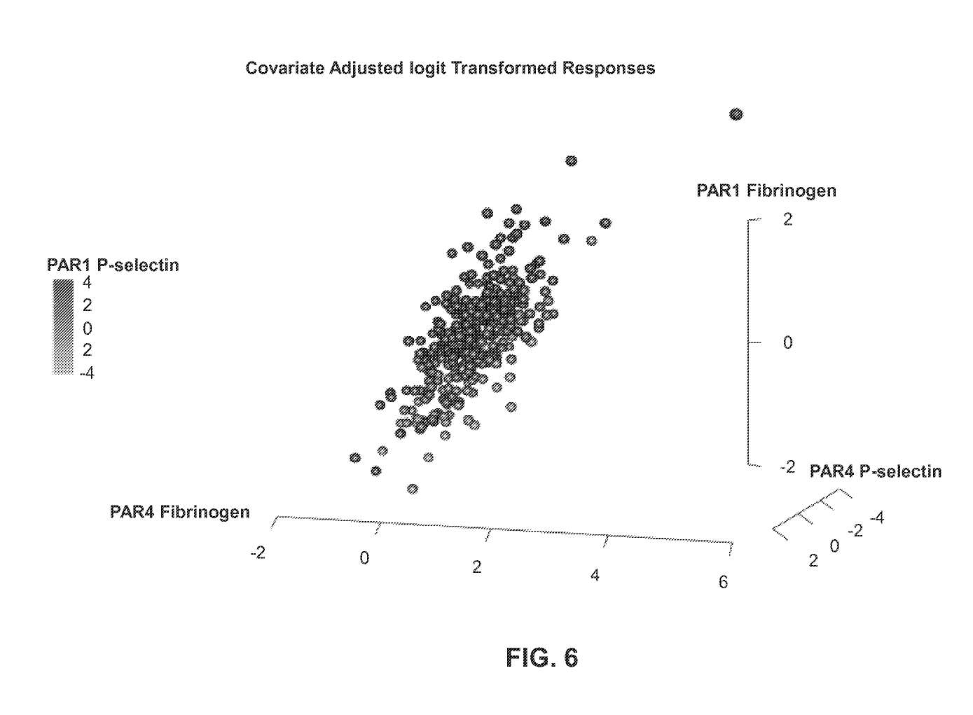

FIG. 6 shows the relationship between the adjusted responses to the two agonists on a single plot.

### Part B

### Introduction

Part B study was conducted in three stages, stage (i) focused on the optimization of platelet inhibition concentration-response curve in response to fibrinogen binding and P-selectin expression. Stage (ii) was data analysis of the 501 subjects tested in Part A, this data was used to identify individuals with hypo-, hyper- and mid-range responses to fibrinogen binding and P-selectin expression.

### Concentration-Response Curves

UDM-002555 was tested at a range of 0.187-6 nM (final concentration) which produced a detailed concentration-response curve for inhibiting platelet activation response (data not shown).

This range was then tested on 12 subjects for both P-selectin expression and fibrinogen binding to observe if inter-individual variation in response to inhibitor platelet activation could be observed.

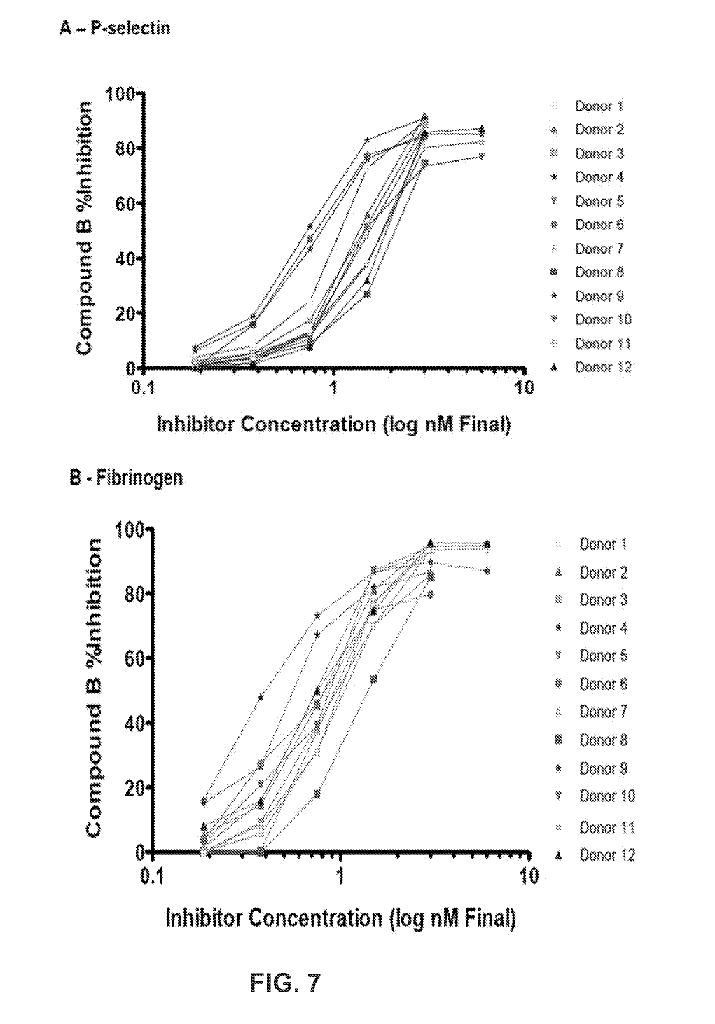

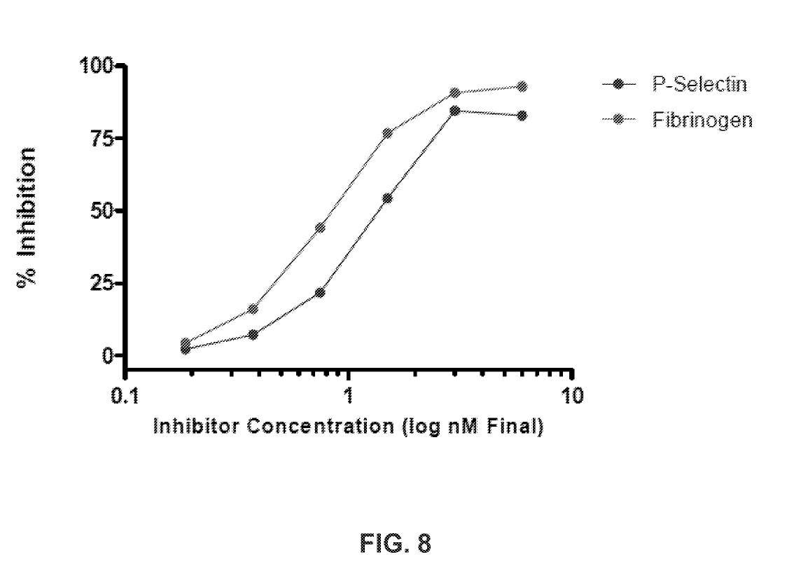

As illustrated in FIG. 7, the selected concentration range gave a detailed concentration-response curve for both fibrinogen binding and P-selectin expression consistently across all 12 donors. This range also enabled the variation in response between individuals to be observed. In addition, the percentage inhibition observed using fibrinogen binding and P-selectin were within a similar inhibition concentration range (FIG. 8).

### Recal of Individuals

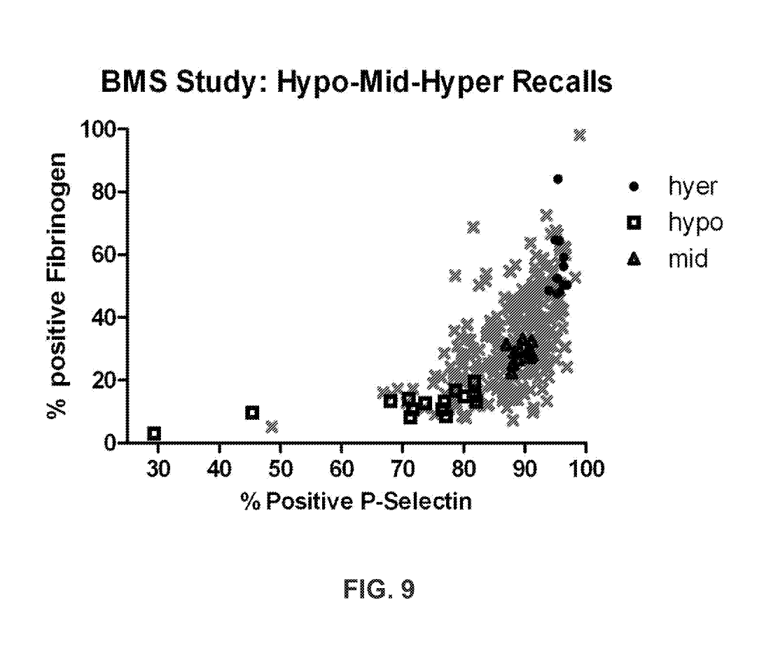

For stage (iii), individuals identified, from stage (ii), as hyper-, mid-range or hypo-responders to PAR4 were recalled. 14 hyper, 13 mid and 14 hypo responders to PAR4 agonist have been recalled. FIG. 9 is a plot of P-Selectin and fibrinogen binding response in the recallable individuals from Part A of the study. In circles are the hyper responders, triangles, the mid and squares the hypo responders that have been successfully recalled for repeat and inhibitor testing.

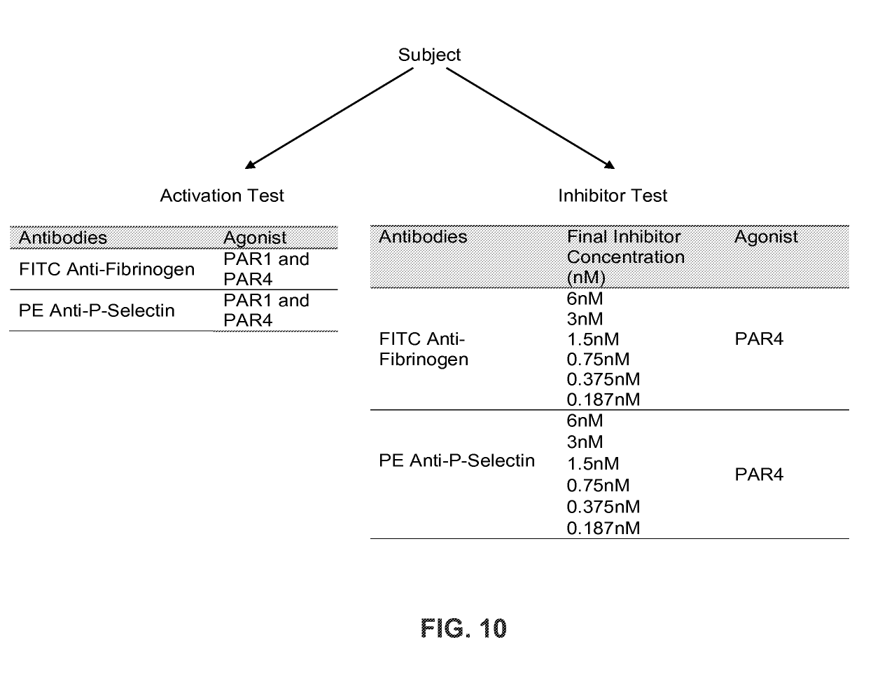

For each recalled subject two sets of tests were performed. The platelet activation in response to PAR1 and PAR4 measurement was repeated. The inhibition of platelet activation was also measured with the PAR4 agonist using the PAR4 inhibitor UDM-2555. A schematic of the testing performed is shown in FIG. 10.

Part B (iii) Recall Methods

### Subjects

Platelet responses from Part A (1st bleed) were ranked to generate a list of the top hyper-, mid-range and hypo-responders to PAR4. Participants were recalled through the National Health Service Blood and Transplant and Cambridge BioResource after gaining informed, written consent (2nd bleed). These donors are healthy individuals.

### Blood Collection

Blood was drawn from the antecubital fossa, contra lateral to the one used for routine whole blood or platelet donation. A 21-gauge butterfly needle and a Vacuette tube (Greiner bio-one, Stonehouse, Gloucestershire, UK) were used following a standardised protocol. The first 3 mL of blood were discarded and a subsequent sample was taken into 3.2% sodium citrate for platelet function analysis.

### Platelet Activation Reagents

PAR1 and PAR4 were supplied as freeze dried peptides and reconstituted with HEPES buffered saline (HBS, 0.14M NaCl, 5 mM KCl, 1 mM MgSO4, 10 mM HEPES (sodium salt), pH 7.4) to 1 mM concentrations prior to storing in single use aliquots at −80° C. Immediately prior to use PAR1 was thawed and diluted in HBS to 10 μM as a working concentration to give a final assay concentration of 1 μM. Similarly PAR4 was diluted to 100 μM working concentration to give a final assay concentration of 10 μM.

The inhibitors, aspirin, apyrase, and hirudin, were all from Sigma Chemical Co. Ltd (Poole, Dorset, UK) and were stored at −80° C. in single-use aliquots until added to the citrated blood during the activation assays.

The antibodies used for flow cytometry were: rabbit FITC-anti-fibrinogen (Dako Ltd, Ely, UK), PE-anti-CD62P and a relevant PE-isotype control (Bristol Institute for Transfusion Science, Bristol, UK). HBS was used for all dilutions.

### Platelet Activation Measurements Using Whole Blood Flow Cytometry

Blood was processed within 15 minutes of venesection, with 5 μL of citrated whole blood containing aspirin (100 μM) and hirudin (10 U/mL) being added to 45 μL of HBS containing apyrase (4 U/mL), either FITC-anti-fibrinogen or PE-anti-CD62P, and either PAR1 (1 μM final concentration) or PAR4 (10 μM final concentration). After 20 minute incubation at room temperature, reactions were stopped by 100-fold dilution in formyl saline (0.2% formaldehyde in 0.9% NaCl, both from Sigma) prior to flow cytometric analysis using either an EPICS Profile XL or FC500 flow cytometer (Beckman-Coulter Ltd., High Wycombe, UK). Platelets were identified by light scatter and results were recorded as the percentage of platelets positive for the relevant activation marker.

Negative controls for the P-selectin antibody were set using an appropriate isotype control and for anti-fibrinogen using samples incubated with the antibody in the presence of 10 mM EDTA.

### Inhibition of Platelet Activation Methods

Inhibition of platelet activation was assessed by incubating 450 μL of citrated whole blood for 5 minutes with the PAR4 inhibitor UDM-2555 at varying concentrations (final concentration range 0.187-6 nM) prior to testing the platelet response to PAR4 agonist as above. 5 μL of blood, pre-incubated with or without the inhibitor, were added to 45 μL of HBS containing apyrase (4 U/mL), PAR4 (10 μM) and either FITC-anti-fibrinogen or PE-anti-CD62P. Platelet activation was measured as platelet-bound fibrinogen and P-selectin expression (CD62P).

Percentage inhibition was calculated as: ((% positive platelets with agonist−positive platelets with agonist and inhibitor)/(% positive platelets with agonist))×100

A log (inhibitor concentration) vs response curve (four-parameter concentration-response curve) was created using Prism software to calculate the IC₅₀ of each donor. This model (shown below) assumes that the data has been normalised, so forces the curve to run from 100% down to 0%.

Model: Y=Bottom+(Top−Bottom)/(1+10̂((Log IC50−X)*HillSlope))

### QC of Platelet Response and Inhibitor Data

All tests were performed in duplicate. The variation between duplicate measurements was accessed by estimating the coefficient of variation. All data points within this analysis had % CVs between duplicates of less than the 20% threshold that was used.

Part B (iii) Reproducibility of Platelet Activation by PAR4

Fibrinogen binding and P-Selectin expression in response to PAR4 was measured in the healthy recalled subjects. The mean time between the two bleeds in these individuals was 256 days (range=100 to 480 days).

The % positive responses for the recalled individuals shown in Table 6.

TABLE 6

| | Percent Positive Responses | |
|---|---|---|
| Type of Responder | Fibrinogen Binding | P-Selectin Expression |
| Lowest Hyper Responder | 47.58% | 93.94% |
| Highest Hypo Responder | 19.54% | 82.07% |
| Highest Mid and Lowest | 22.44% to 33.12% | 86.995% to 91.175% |
| Mid Responder | | |

The % positive values are in Tables 7A and B.

TABLE 7A
Fibrinogen Binding

| | | | 2^{nd} bleed PAR4 - |
|---|---|---|---|
| | PAR4 respondor | 1^{st} bleed PAR4 - | % Positive |
| Sample ID | catergory | % Positive Platelets | Platelets |
| C0EVUO | Hyper | 84.160 | 87.900 |
| C0ETTY | Hyper | 56.295 | 82.340 |
| C0FE1V | Hyper | 59.115 | 63.045 |
| C0FQR5 | Hyper | 64.700 | 52.280 |
| C0FRAZ | Hyper | 52.425 | 63.375 |
| C0FHGQ | Hyper | 64.350 | 48.100 |
| C0FS0E | Hyper | 50.475 | 53.545 |
| C0FHR4 | Hyper | 52.435 | 51.125 |
| C0EW4Z | Hyper | 47.580 | 51.270 |
| C0EW5X | Hyper | 48.585 | 46.465 |
| C0FW5P | Hyper | 48.000 | 34.735 |
| C0FVF9 | Hyper | 38.11 | 69.155 |
| C0FVH5 | Hyper | 66.645 | 82.075 |
| C0FY1P | Hyper | 50.39 | 50.505 |
| C0FHEU | Mid-range | 31.505 | 56.845 |
| C0FH4D | Mid-range | 29.130 | 49.160 |
| C0EXXA | Mid-range | 27.350 | 44.980 |
| C0F0DQ | Mid-range | 33.120 | 36.345 |
| C0F0CS | Mid-range | 29.760 | 36.435 |
| C0FFT8 | Mid-range | 22.440 | 40.425 |
| C0FRFP | Mid-range | 26.635 | 21.445 |
| C0FWQK | Mid-range | 29 | 23.015 |
| C0FXAB | Mid-range | 32.56 | 49.465 |
| C0G0WH | Mid-range | 28.23 | 30.655 |
| C0G2QL | Mid-range | 29.915 | 28.795 |
| C0G8WM | Mid-range | 32.26 | 52.275 |
| C0G8XK | Mid-range | 25.245 | 22.26 |
| C0FRMB | Hypo | 16.705 | 35.870 |
| C0FHHO | Hypo | 15.005 | 30.735 |
| C0FVY8 | Hypo | 13.215 | 29.380 |
| C0F5E4 | Hypo | 14.910 | 20.545 |
| C0F3K0 | Hypo | 3.005 | 31.445 |
| C0F7S5 | Hypo | 14.050 | 19.570 |
| C0FW0Z | Hypo | 13.365 | 19.145 |
| C0EX6R | Hypo | 8.250 | 21.085 |
| C0FJQZ | Hypo | 10.695 | 18.135 |
| C0ETWS | Hypo | 8.630 | 17.165 |
| C0EVWK | Hypo | 10.820 | 13.155 |
| C0G4YY | Hypo | 12.68 | 25.26 |

TABLE 7B
P-selectin expression

| | PAR4 respondor | 1^{st} bleed PAR4 - | 2^{nd} bleed PAR4 - |
|---|---|---|---|
| Sample ID | catergory | % Positive Platelets | % Positive Platelets |
| C0FS0E | Hyper | 96.255 | 96.985 |
| C0ETTY | Hyper | 96.340 | 95.840 |
| C0FE1V | Hyper | 96.345 | 95.680 |
| C0FHGQ | Hyper | 95.620 | 95.500 |
| C0EVUO | Hyper | 95.455 | 95.550 |
| C0FHR4 | Hyper | 95.325 | 94.960 |
| C0FRAZ | Hyper | 95.225 | 94.195 |
| C0FW5P | Hyper | 95.725 | 91.470 |
| C0FQR5 | Hyper | 94.965 | 91.965 |
| C0EW4Z | Hyper | 95.300 | 91.285 |
| C0EW5X | Hyper | 93.940 | 87.715 |
| C0FVF9 | Hyper | 93.405 | 96.870 |
| C0FVH5 | Hyper | 94.275 | 96.155 |
| C0FY1P | Hyper | 96.955 | 95.280 |
| C0F0DQ | Mid-range | 89.660 | 93.710 |
| C0FH4D | Mid-range | 90.430 | 92.910 |
| C0FHEU | Mid-range | 86.995 | 95.205 |
| C0F0CS | Mid-range | 89.085 | 92.505 |
| C0FFT8 | Mid-range | 87.820 | 88.270 |
| C0EXXA | Mid-range | 90.800 | 80.780 |
| C0FRFP | Mid-range | 88.920 | 76.835 |
| C0FWQK | Mid-range | 88.310 | 90.610 |
| C0FXAB | Mid-range | 91.100 | 91.140 |
| C0G0WH | Mid-range | 91.175 | 93.725 |
| C0G2QL | Mid-range | 90.555 | 90.305 |
| C0G8WM | Mid-range | 89.795 | 92.040 |
| C0G8XK | Mid-range | 88.075 | 89.190 |
| C0FHHO | Hypo | 81.805 | 92.800 |
| C0FVY8 | Hypo | 82.070 | 86.955 |
| C0F5E4 | Hypo | 80.095 | 85.025 |
| C0FRMB | Hypo | 78.695 | 85.175 |
| C0FJQZ | Hypo | 76.515 | 86.030 |
| C0EX6R | Hypo | 71.285 | 90.360 |
| C0F7S5 | Hypo | 71.000 | 83.080 |
| C0EVWK | Hypo | 71.720 | 77.415 |
| C0ETWS | Hypo | 77.115 | 71.880 |
| C0FW0Z | Hypo | 68.045 | 69.540 |
| C0F3K0 | Hypo | 29.370 | 40.835 |
| C0G4YY | Hypo | 73.690 | 81.385 |

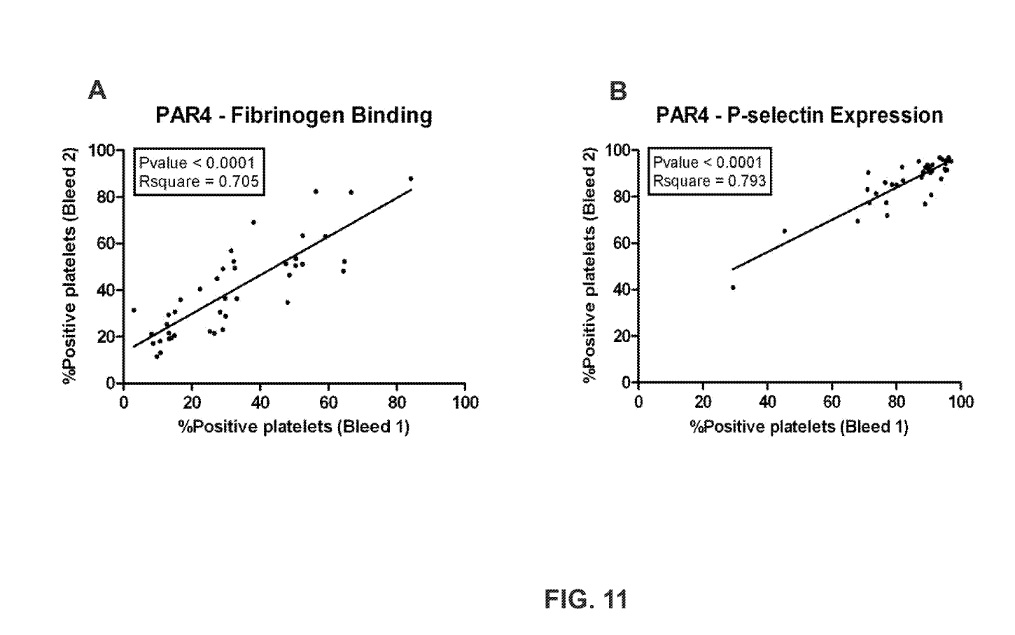

The repeat measurements of percent positive platelet responses to PAR4 were associated indicating reproducibility of results in the Fibrinogen binding and the P-Selectin assays (FIGS. 11A and 11B).

Part B (iii) Platelet Inhibition Results

The inhibition of the PAR4 response was tested in platelets using the inhibitor UDM-2555. Previous testing determined that a range of 0.187-6 nM (final concentration) of inhibitor was appropriate in our assays.

Recalled donors were tested using the methods as described above. Tables 8A and 8B contain the percentage inhibition for each recalled donor and the IC₅₀ value calculated using the Fibrinogen binding and the P-Selectin expression read outs.

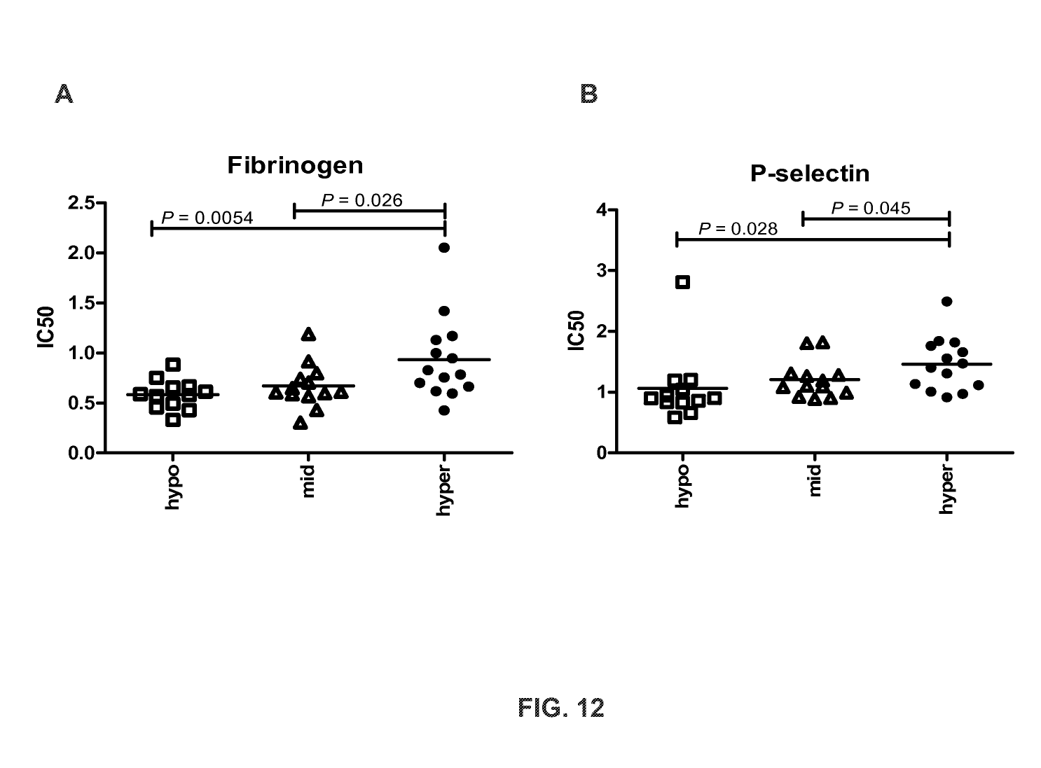

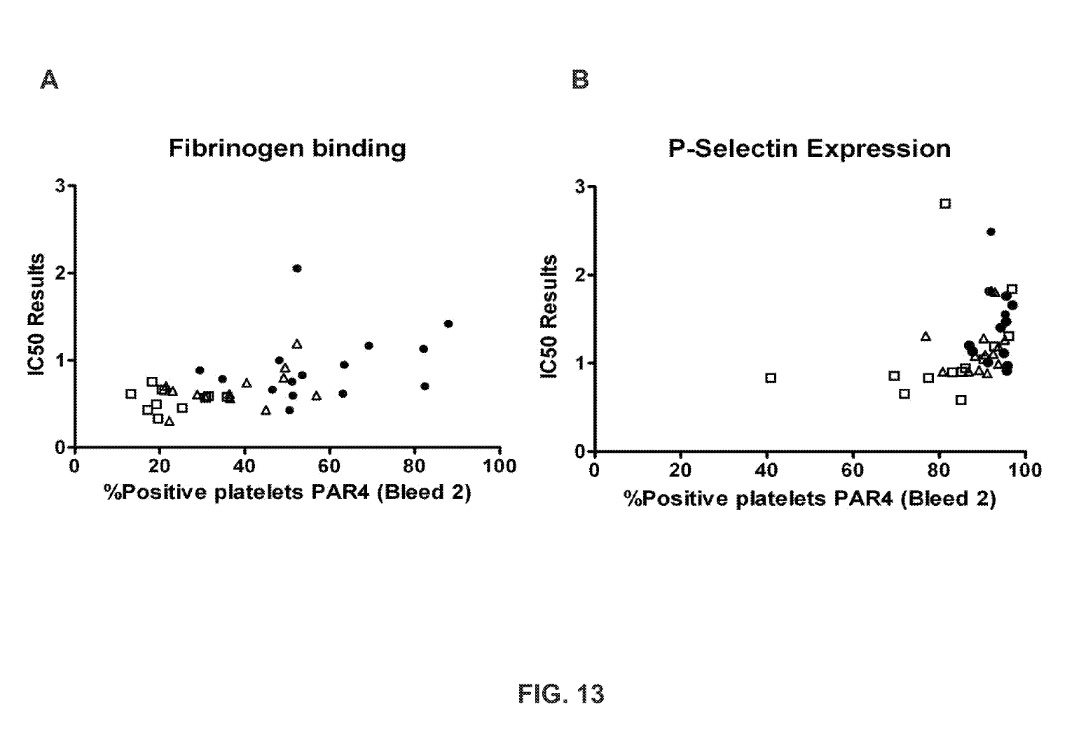

FIGS. 12A and 12B show the IC50 results for the hypo, mid and hyper responder groups. FIGS. 13A and 13B show the IC50 results plotted against the Bleed 2 percent positive platelet response using Fibrinogen binding or P-Selectin expression. Both plots show a positive association between these two values with the high responders requiring high concentrations of inhibitor for 50% inhibition and the low responders requiring less inhibitor for 50% inhibition.

TABLE 8A
Fibrinogen Binding % Inhibition

| | | Inhibitor Final Concentrations (nM) | | | | | | |
|---|---|---|---|---|---|---|---|---|
| | | 0.187 | 0.375 | 0.750 | 1.500 | 3.000 | 6.000 | |
| | | Inhibitor Final Concentration (Log (X)nM) | | | | | | |
| Sample ID | Bleed 1 classification for PAR4 response | −0.728 | −0.426 | −0.125 | 0.176 | 0.477 | 0.778 | IC50 |
| C0FQR5 | Hyper | 3.360061 | 4.876452 | 13.66934 | 30.96056 | 77.69051 | 96.0672 | 2.054 |
| C0EVUO | Hyper | 7.27933 | 13.17438 | 32.22275 | 50.67109 | 85.56767 | 91.99958 | 1.419 |
| C0FVF9 | Hyper | 12.29296 | 21.01377 | 31.14482 | 68.98823 | 88.80463 | 94.86463 | 1.169 |
| C0FVH5 | Hyper | 4.889646 | 8.669875 | 20.09274 | 74.80629 | 94.52924 | 96.30195 | 1.131 |
| C0FHGQ | Hyper | 14.88773 | 10.5155 | 42.40987 | 69.8055 | 89.94308 | 95.48545 | 0.9996 |
| C0FRAZ | Hyper | 10.7069 | 28.54697 | 39.33947 | 68.23596 | 87.88073 | 89.06484 | 0.9474 |
| C0FS0E | Hyper | −4.724834 | 10.79852 | 39.17065 | 64.4674 | 91.15154 | 91.86064 | 0.8279 |
| C0FW5P | Hyper | 13.94291 | 19.69749 | 52.82611 | 77.03855 | 93.08541 | 94.7117 | 0.7852 |
| C0FHR4 | Hyper | 14.82865 | 24.96031 | 53.96395 | 86.80549 | 94.68671 | 95.70454 | 0.7555 |
| C0ETTY | Hyper | 3.81292 | 18.46112 | 52.81169 | 88.71211 | 96.06364 | 96.09107 | 0.7018 |
| C0EW5X | Hyper | 3.426261 | 23.62205 | 50.56395 | 82.65588 | 93.22196 | 94.27538 | 0.6635 |
| C0FE1V | Hyper | 9.854285 | 23.8989 | 63.86762 | 91.61933 | 94.03145 | 94.73121 | 0.6176 |
| C0EW4Z | Hyper | 9.211843 | 28.87577 | 60.68214 | 88.48769 | 95.52911 | 94.55891 | 0.5956 |
| C0FY1P | Hyper | 11.34557 | 37.84065 | 72.65329 | 85.07759 | 96.21499 | 95.69455 | 0.427 |
| C0G8WM | Mid-range | 6.462802 | 14.55043 | 33.41853 | 61.04062 | 89.21954 | 95.17116 | 1.191 |
| C0FXAB | Mid-range | 16.88628 | 29.4851 | 37.97892 | 94.04014 | 96.62477 | 95.28684 | 0.916 |
| C0FH4D | Mid-range | 2.522845 | 22.83472 | 41.63687 | 64.28288 | 88.76639 | 92.86849 | 0.7997 |
| C0FFT8 | Mid-range | 7.876511 | 21.81007 | 50.15847 | 86.39512 | 93.56732 | 94.24815 | 0.7417 |
| C0FRFP | Mid-range | −0.1673748 | 13.29986 | 47.37994 | 76.43878 | 89.80301 | 89.84164 | 0.7042 |
| C0FWQK | Mid-range | 19.86931 | 40.00409 | 54.87033 | 85.54217 | 90.83112 | 91.21912 | 0.65 |
| C0F0DQ | Mid-range | 9.98811 | 27.33353 | 58.54638 | 84.34899 | 92.28597 | 91.78062 | 0.6121 |
| C0G2QL | Mid-range | 12.06266 | 25.81375 | 59.93037 | 79.33855 | 89.34726 | 88.25066 | 0.6054 |
| C0FHEU | Mid-range | 4.143326 | 25.46842 | 56.80896 | 87.10643 | 93.85744 | 93.73189 | 0.5975 |
| C0G0WH | Mid-range | −2.614686 | 23.29732 | 44.6532 | 75.43448 | 88.61751 | 90.91905 | 0.5875 |
| C0F0CS | Mid-range | −4.779833 | 22.57325 | 51.13766 | 85.39859 | 93.56687 | 93.95973 | 0.5669 |
| C0EXXA | Mid-range | 20.38385 | 45.37392 | 72.44209 | 89.76836 | 93.31568 | 92.30973 | 0.4301 |
| C0G8XK | Mid-range | 12.51504 | 48.8568 | 74.38327 | 92.02768 | 93.84777 | 93.0355 | 0.3044 |
| C0FVY8 | Hypo | 12.30284 | 18.17823 | 34.54259 | 78.96294 | 71.88486 | 91.0489 | 0.8846 |
| C0FJQZ | Hypo | 18.52651 | 21.39334 | 50.06165 | 80.73367 | 79.83971 | 82.95315 | 0.7519 |
| C0F5E4 | Hypo | −21.46577 | −16.29701 | 50.39537 | 86.6731 | 91.6297 | 90.62681 | 0.6654 |
| C0EX6R | Hypo | 15.39267 | 25.4712 | 55.34031 | 77.35602 | 84.24084 | 83.40314 | 0.6574 |
| C0EVWK | Hypo | 7.41688 | 19.32773 | 57.14286 | 79.75886 | 84.83741 | 83.85093 | 0.614 |
| C0F3K0 | Hypo | 11.71875 | 31.91964 | 42.07589 | 70.14509 | 73.15849 | 72.65625 | 0.5887 |
| C0FRMB | Hypo | 0.3418272 | 25.13984 | 52.22188 | 86.01616 | 91.19018 | 91.26787 | 0.5806 |
| C0FHHO | Hypo | −4.629464 | 20.59932 | 51.911 | 84.33519 | 90.41809 | 92.87637 | 0.5697 |
| C0FW0Z | Hypo | −26.2734 | 9.192135 | 40.3222 | 76.49846 | 87.18313 | 84.38759 | 0.4939 |
| C0G4YY | Hypo | −4.060289 | 23.59274 | 40.32605 | 70.28606 | 78.56044 | 86.0043 | 0.4546 |
| C0ETWS | Hypo | −29.10356 | 9.455587 | 51.69873 | 78.18256 | 80.59763 | 78.87843 | 0.4287 |
| C0F7S5 | Hypo | 23.14209 | 49.8909 | 71.32589 | 86.39455 | 88.87177 | 90.39918 | 0.3314 |

TABLE 8B
P-selectin Expression % inhibition

| | | Inhibitor Final Concentrations (nM) | | | | | | |
|---|---|---|---|---|---|---|---|---|
| | | 0.187 | 0.375 | 0.750 | 1.500 | 3.000 | 6.000 | |
| | | Inhibitor Final Concentration (Log (X)nM) | | | | | | |
| Sample ID | Bleed 1 classification for PAR4 response | −0.728 | −0.426 | −0.125 | 0.176 | 0.477 | 0.778 | IC50 |
| C0FQR5 | Hyper | −0.9378543 | −0.4070906 | 1.138823 | 11.07905 | 60.17727 | 86.95764 | 2.49 |
| C0FVF9 | Hyper | 0.7315817 | 1.447707 | 4.44101 | 29.24266 | 77.10973 | 88.39258 | 1.841 |
| C0FW5P | Hyper | 0.434663 | 1.276479 | 16.91334 | 28.70426 | 90.09628 | 93.18845 | 1.819 |
| C0EVUO | Hyper | 0.4747496 | 2.556344 | 8.357679 | 27.48852 | 49.39482 | 61.26878 | 1.762 |
| C0FS0E | Hyper | 0.9794269 | 2.326787 | 10.50422 | 35.56511 | 90.64622 | 90.21609 | 1.659 |
| C0FY1P | Hyper | 1.22321 | 3.62781 | 18.43701 | 42.25823 | 88.73497 | 89.05384 | 1.554 |
| C0FHGQ | Hyper | 2.337731 | 2.870713 | 12.80739 | 47.59895 | 87.9314 | 89.51979 | 1.473 |
| C0FRAZ | Hyper | 3.296173 | 7.878658 | 16.28256 | 50.44485 | 87.41559 | 85.30925 | 1.403 |
| C0FVH5 | Hyper | 1.465107 | 3.354732 | 8.70263 | 59.96583 | 90.17395 | 89.20066 | 1.309 |
| C0EW5X | Hyper | 2.787627 | 7.970105 | 25.05046 | 62.9862 | 89.47684 | 89.4823 | 1.135 |
| C0FHR4 | Hyper | 2.735271 | 6.883498 | 19.85604 | 68.28046 | 88.71234 | 88.755 | 1.114 |
| C0EW4Z | Hyper | 1.680392 | 10.22702 | 29.07857 | 64.06076 | 85.6666 | 85.02114 | 1.01 |
| C0ETTY | Hyper | 0.2092378 | 3.53612 | 22.72323 | 71.65873 | 82.53387 | 84.93488 | 0.9728 |
| C0FE1V | Hyper | 2.9785 | 9.391715 | 32.03985 | 82.35448 | 92.61143 | 92.5118 | 0.9147 |
| C0G8WM | Mid-range | 0.4187789 | 1.564911 | 9.135993 | 33.9762 | 71.72691 | 86.74785 | 1.822 |
| C0FH4D | Mid-range | 3.696244 | 2.847033 | 10.22863 | 30.1742 | 66.35275 | 76.69025 | 1.805 |
| C0FRFP | Mid-range | 2.765199 | 6.876773 | 19.19942 | 56.30622 | 87.10983 | 91.28178 | 1.309 |
| C0G2QL | Mid-range | 4.669497 | 8.677435 | 27.36645 | 54.92585 | 88.14157 | 91.19025 | 1.283 |
| C0FHEU | Mid-range | 2.501701 | 6.044905 | 17.79976 | 60.52755 | 90.74162 | 92.42686 | 1.264 |
| C0G0WH | Mid-range | 3.190555 | 7.715541 | 21.53213 | 60.14827 | 85.20593 | 88.22625 | 1.193 |
| C0F0CS | Mid-range | 4.382841 | 10.50786 | 25.07533 | 68.42163 | 89.76059 | 91.43703 | 1.106 |
| C0FFT8 | Mid-range | 4.525742 | 8.501057 | 19.14267 | 67.54698 | 80.75169 | 83.73179 | 1.083 |
| C0F0DQ | Mid-range | −3.573302 | 1.824541 | 27.68289 | 67.52551 | 90.15447 | 91.30283 | 0.9909 |
| C0G8XK | Mid-range | 4.409073 | 17.79713 | 32.82125 | 80.47252 | 89.73989 | 88.2813 | 0.922 |
| C0EXXA | Mid-range | 4.246692 | 13.88711 | 35.97952 | 77.12334 | 90.75437 | 91.19185 | 0.9046 |
| C0FXAB | Mid-range | 4.267408 | 11.47848 | 33.29304 | 80.72413 | 88.08632 | 88.00169 | 0.8872 |
| C0FWQK | Mid-range | 3.464735 | 12.51123 | 28.48158 | 67.4416 | 92.25067 | 92.74483 | 1.096 |
| C0G4YY | Hypo | 1.322065 | 7.250226 | 24.65439 | 45.01056 | 66.64655 | 91.71748 | 2.808 |
| C0FVY8 | Hypo | 5.133554 | 11.66927 | 21.66406 | 54.85254 | 78.30697 | 78.79947 | 1.202 |
| C0FHHO | Hypo | 3.238432 | 5.935338 | 27.23178 | 59.06386 | 88.1883 | 92.43472 | 1.191 |
| C0EX6R | Hypo | 4.065773 | 12.45073 | 29.53035 | 65.44093 | 88.0955 | 87.04246 | 1.046 |
| C0FJQZ | Hypo | 8.145123 | 17.50961 | 36.82124 | 76.20135 | 91.39236 | 91.58457 | 0.942 |
| C0FRMB | Hypo | 0.6501726 | 10.86881 | 31.65708 | 78.5443 | 88.02071 | 88.9183 | 0.9005 |
| C0F7S5 | Hypo | 4.209032 | 14.61517 | 34.26867 | 80.18177 | 88.87816 | 89.15081 | 0.8969 |
| C0FW0Z | Hypo | 4.245283 | 19.33962 | 39.60183 | 74.81964 | 91.47475 | 90.57991 | 0.8577 |
| C0EVWK | Hypo | 6.751184 | 13.68084 | 40.64865 | 77.16167 | 88.23927 | 87.5377 | 0.8341 |
| C0F3K0 | Hypo | 13.359 | 30.50048 | 43.61887 | 77.05486 | 88.49856 | 87.31473 | 0.8328 |
| C0ETWS | Hypo | 10.11463 | 21.83262 | 54.85128 | 81.9435 | 86.29655 | 85.77958 | 0.656 |
| C0F5E4 | Hypo | 1.962965 | 6.305955 | 70.41094 | 81.34349 | 84.91909 | 87.02107 | 0.585 |

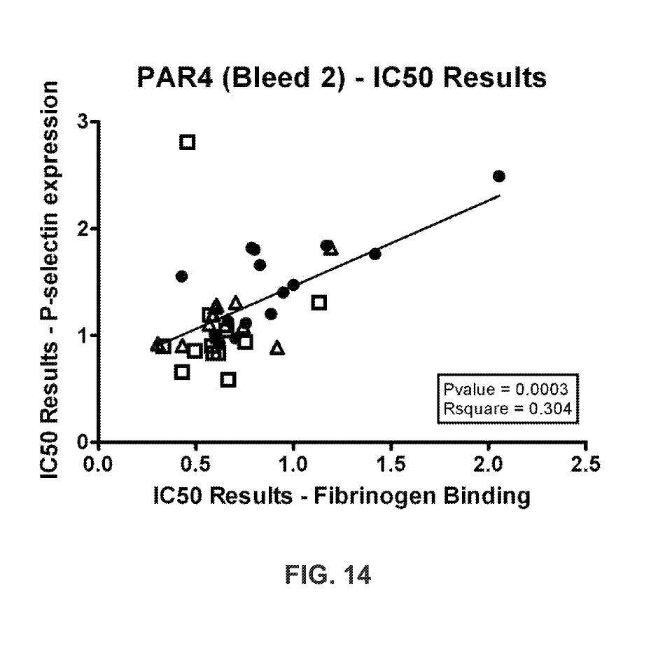

FIG. 14 shows the association between the 1050 measured using fibrinogen binding and P-Selectin expression. Circles are the hyper responders, triangles, the mid and squares the hypo responders.

### CONCLUSIONS

The results from the reproducibility study indicate that an individual's platelet response to PAR4 agonist is stable and does not change over time in a healthy individual. The result also indicate that a diagnostic assay can be utilized to identify poor responders to PAR4 agonist & enrich PAR-4 responding patients who might benefit most from a PAR4 antagonist treatment.

The results from the inhibitor testing indicate that, in a healthy individual, the platelet response to a PAR4 agonist is positively associated with the amount of PAR4 inhibitor, UDM-2555, required to inhibit this response. The result also indicate that Potential application for using a diagnostic assay to monitor pharmacodynamic effect of PAR4 antagonist and personalize treatment regimen to achieve optimal benefit for the patients

### REFERENCES CITED IN EXAMPLE 4

Antithrombotic Trialists, C., 2002: Collaborative meta-analysis of randomised trials of antiplatelet therapy for prevention of death, myocardial infarction, and stroke in high risk patients. Bmj, 324, 71-86.
Fontana, P. et al. 2003: Adenosine diphosphate-induced platelet aggregation is associated with P2Y12 gene sequence variations in healthy subjects. Circulation, 108, 989-995.
Gieger, C. et al. 2011: New gene functions in megakaryopoiesis and platelet formation. Nature, 480, 201-208.
Hamm, C. W. et al. 2011: ESC Guidelines for the management of acute coronary syndromes in patients presenting without persistent ST-segment elevation. Eur Heart J, 32, 2999-3054.
Hetherington, S. L. et al. 2005: Dimorphism in the P2Y1 ADP receptor gene is associated with increased platelet activation response to ADP. Arterioscler Thromb Vasc Biol, 25, 252-257.
Johnson, A. D. et al. 2010: Genome-wide meta-analyses identifies seven loci associated with platelet aggregation in response to agonists. Nat Genet, 42, 608-613.
Jones, C. I. et al. 2009: A functional genomics approach reveals novel quantitative trait loci associated with platelet signaling pathways. Blood, 114, 1405-1416.
Jones, C. I. et al. 2007: Mapping the platelet profile for functional genomic studies and demonstration of the effect size of the GP6 locus. J Thromb Haemost, 5, 1756-1765.
O'Donnell, C. J. et al. 2001: Genetic and environmental contributions to platelet aggregation: the Framingham heart study. Circulation, 103, 3051-3056.
Panzer, S. et al. 2006: Agonists-induced platelet activation varies considerably in healthy male individuals: studies by flow cytometry. Ann Hematol, 85, 121-125.
Serebruany, V. L. et al. 2005: Variability in platelet responsiveness to clopidogrel among 544 individuals. Journal of the American College of Cardiology, 45, 246-251.
Soranzo, N. et al. 2009a: A novel variant on chromosome 7q22.3 associated with mean platelet volume, counts, and function. Blood, 113, 3831-3837.
Soranzo, N. et al. 2009b: A genome-wide meta-analysis identifies 22 loci associated with eight hematological parameters in the HaemGen consortium. Nat Genet, 41, 1182-1190.
Steg, P. G. et al. 2013: 2012 ESC STEMI guidelines and reperfusion therapy: Evidence-based recommendations, ensuring optimal patient management. Heart, 99, 1156-1157.
Tofler, G. H. et al. 1987: Concurrent morning increase in platelet aggregability and the risk of myocardial infarction and sudden cardiac death. N Engl J Med, 316, 1514-1518.
Trip, M. D. et al. 1990: Platelet hyperreactivity and prognosis in survivors of myocardial infarction. N Engl J Med, 322, 1549-1554.
Yee, D. L. et al. 2005: Aggregometry detects platelet hyperreactivity in healthy individuals. Blood, 106, 2723-2729.

The breadth and scope of the present disclosure should not be limited by any of the above-described exemplary aspects, but should be defined only in accordance with the following claims and their equivalents.

This application claims benefit to U.S. Provisional Patent Application 61/895,968, filed Oct. 25, 2013, the entirety of which is hereby incorporated herein.

All patents and publications referred to herein are expressly incorporated by reference in their entireties.

What is claimed is:

1. A method for identifying a PAR4 agonist responder comprising contacting a PAR4 agonist with platelets in a sample obtained from a subject in need thereof, wherein the PAR4 agonist activates the platelets and comprises an amino acid sequence of Formula I:
Ala-Xₐₐ₁-Pro-Gly-Xₐₐ₂-Leu-Val (Formula I)
wherein,
the amino terminus of the peptide is not fused to an amino acid;
Xₐₐ₁ is selected from Tyr or Phe(4-F);
Xₐₐ₂ is selected from Trp(5-OH), (D,L)-Trp(5-Br), D-Trp, Bzt, Tpi, His, Tza, 3-Thi, 3-Fur, His(Bzl), Phe, Tyr, Phe(penta-F), 2-Pya, 3-Pya, 4-Pya, Dpa, 3-Pya(4-Tolyl), Bip(2-Methyl), 1-Naphthyl-Ala, 2-Naphthyl-Ala, Tyr(Bzl), or Styryl-Ala; and
(−) is a peptide bond; and
the peptide has a PAR4 agonist activity.

2. A method for identifying a PAR4 antagonist therapy responder comprising contacting a PAR4 agonist with platelets in a sample obtained from a subject in need of PAR4 antagonist therapy, wherein the PAR4 agonist activates the platelets and comprises an amino acid sequence of Formula I:
Ala-Xₐₐ₁-Pro-Gly-Xₐₐ₂-Leu-Val (Formula I)
wherein,
the amino terminus of the peptide is not fused to an amino acid;
Xₐₐ₁ is selected from Tyr or Phe(4-F);
Xₐₐ₂ is selected from Trp(5-OH), (D,L)-Trp(5-Br), D-Trp, Bzt, Tpi, His, Tza, 3-Thi, 3-Fur, His(Bzl), Phe, Tyr, Phe(penta-F), 2-Pya, 3-Pya, 4-Pya, Dpa, 3-Pya(4-Tolyl), Bip(2-Methyl), 1-Naphthyl-Ala, 2-Naphthyl-Ala, Tyr(Bzl), or Styryl-Ala; and
(−) is a peptide bond; and
the peptide has a PAR4 agonist activity, and
wherein platelet activation indicates that the subject is a PAR4 antagonist therapy responder.

3. The method of claim 1 or 2, which further comprises measuring platelet activation after the platelets have been in contact with the PAR4 agonist.

4. A method for evaluating platelet activation by a PAR4 agonist in a subject in need thereof comprising (i) contacting a PAR4 agonist with platelets in a sample obtained from the subject, wherein the PAR4 agonist activates the platelets and comprises an amino acid sequence of Formula I:
Ala-Xₐₐ₁-Pro-Gly-Xₐₐ₂-Leu-Val (Formula I)
wherein,
the amino terminus of the peptide is not fused to an amino acid;
Xₐₐ₁ is selected from Tyr or Phe(4-F);
Xₐₐ₂ is selected from Trp(5-OH), (D,L)-Trp(5-Br), D-Trp, Bzt, Tpi, His, Tza, 3-Thi, 3-Fur, His(Bzl), Phe, Tyr, Phe(penta-F), 2-Pya, 3-Pya, 4-Pya, Dpa, 3-Pya(4-Tolyl), Bip(2-Methyl), 1-Naphthyl-Ala, 2-Naphthyl-Ala, Tyr(Bzl), or Styryl-Ala; and
(−) is a peptide bond; and
the peptide has a PAR4 agonist activity
and (ii) measuring activation of the platelets.

5. A method for evaluating responsiveness of a subject to PAR4 antagonist therapy comprising (i) contacting a PAR4 agonist with platelets in a sample obtained from the subject in need of the therapy, wherein the PAR4 agonist activates the platelets and comprises an amino acid sequence of Formula I:
Ala-Xₐₐ₁-Pro-Gly-Xₐₐ₂-Leu-Val (Formula I)
wherein,
the amino terminus of the peptide is not fused to an amino acid;
Xₐₐ₁ is selected from Tyr and Phe(4-F);
Xₐₐ₂ is selected from Trp(5-OH), (D,L)-Trp(5-Br), D-Trp, Bzt, Tpi, His, Tza, 3-Thi, 3-Fur, His(Bzl), Phe, Tyr, Phe(penta-F), 2-Pya, 3-Pya, 4-Pya, Dpa, 3-Pya(4-Tolyl), Bip(2-Methyl), 1-Naphthyl-Ala, 2-Naphthyl-Ala, Tyr(Bzl), or Styryl-Ala; and
(−) is a peptide bond; and
the peptide has a PAR4 agonist activity, and
(ii) measuring the platelet activation,
wherein platelet activation indicates that the subject is a responder for PAR4 antagonist therapy.

6. The method of any one of claims 1 to 5, wherein the PAR4 agonist activates the platelets equal to or above a normal diagnostic score or below a normal diagnostic score after being in contact with the platelets.

7. The method of any one of claims 1 to 5, wherein the PAR4 agonist activates the platelets equal to or above a high diagnostic score after being in contact with the platelets.

8. The method of any one of claims 3 to 7, which further comprises providing to a healthcare provider the result of measuring the platelet activation.

9. The method of any one of claims 1 to 8, wherein the subject is a normal responder for the PAR4 agonist and is a normal responder for PAR4 antagonist therapy.

10. The method of any one of claims 1 to 8, wherein the subject is a high responder for the PAR4 agonist and is a low responder for PAR4 antagonist therapy.

11. The method of any one of claims 1, 3, and 4, wherein the subject is a low responder for the PAR4 agonist and is a high responder for PAR4 antagonist therapy.

12. The method of any one of claims 1 to 10, which further comprises recommending that a healthcare provider administer a PAR4 antagonist to the subject.

13. The method of any one of claims 1 to 10, which further comprises administering to the subject an effective amount of a PAR4 antagonist.

14. A method for treating a subject with a PAR4 antagonist, the method comprising: (i) obtaining a blood sample from the subject (ii) contacting a PAR4 agonist with platelets in the sample obtained from the subject, (iii) measuring activation of the platelets and (iv) determining if the subject is a high responder, normal responder, low responder or non-responder to PAR4 agonist based on the amount of platelet activation observed, and (v) administering PAR4 antagonist to the subject if the subject is a high responder, normal responder or low responder to PAR4 agonist; wherein the PAR4 agonist comprises an amino acid sequence of Formula I:
Ala-Xₐₐ₁-Pro-Gly-Xₐₐ₂-Leu-Val (Formula I)
wherein,
the amino terminus of the peptide is not fused to an amino acid;
Xₐₐ₁ is selected from Tyr and Phe(4-F);
Xaa2 is selected from Trp(5-OH), (D,L)-Trp(5-Br), D-Trp, Bzt, Tpi, His, Tza, 3-Thi, 3-Fur, His(Bzl), Phe, Tyr, Phe(penta-F), 2-Pya, 3-Pya, 4-Pya, Dpa, 3-Pya(4-Tolyl), Bip(2-Methyl), 1-Naphthyl-Ala, 2-Naphthyl-Ala, Tyr(Bzl), or Styryl-Ala; and
(−) is a peptide bond.

15. The method of claim 14, wherein the subject is a high responder if the subject's diagnostic score is higher than or equal to a high diagnostic score.

16. The method of claim 14, wherein the subject is a low responder if the subject's diagnostic score is lower than a normal diagnostic score.

17. The method of claim 14, wherein the subject is a normal responder if the subject's diagnostic score is higher than or equal to a normal diagnostic score and lower than a high diagnostic score.

18. A method for treating a subject with PAR4 antagonist, the method comprising: (i) obtaining a blood sample from the subject (ii) contacting a PAR4 agonist with platelets in the sample obtained from the subject, (iii) measuring activation of the platelets and (iv) determining if the subject is a high responder, normal responder, low responder or non-responder to PAR4 agonist based on the amount of platelet activation observed, (v) providing a report to a healthcare provider recommending that the healthcare provider treat the subject with PAR4 antagonist if the subject is a high responder, a normal responder or a low responder; wherein the PAR4 agonist comprises an amino acid sequence of Formula I:
Ala-Xₐₐ₁-Pro-Gly-Xₐₐ₂-Leu-Val (Formula I)
wherein,
the amino terminus of the peptide is not fused to an amino acid;
Xₐₐ₁ is selected from Tyr and Phe(4-F);
Xaa2 is selected from Trp(5-OH), (D,L)-Trp(5-Br), D-Trp, Bzt, Tpi, His, Tza, 3-Thi, 3-Fur, His(Bzl), Phe, Tyr, Phe(penta-F), 2-Pya, 3-Pya, 4-Pya, Dpa, 3-Pya(4-Tolyl), Bip(2-Methyl), 1-Naphthyl-Ala, 2-Naphthyl-Ala, Tyr(Bzl), or Styryl-Ala; and
(−) is a peptide bond.

19. The method of claim 18, wherein the subject is a high responder if the subject's diagnostic score is higher than or equal to a high diagnostic score.

20. The method of claim 18, wherein the subject is a low responder if the subject's diagnostic score is lower than a normal diagnostic score.

21. The method of claim 18, wherein the subject is a normal responder if the subject's diagnostic score is higher than or equal to a normal diagnostic score and lower than a high diagnostic score.

22. A method for treating a subject with PAR4 antagonist, the method comprising: (i) obtaining a blood sample from a subject that has not been treated with a PAR4 antagonist, (ii) pre-incubating a first fraction of the sample with a PAR4 antagonist while the second fraction is not pre-incubated with the PAR4 antagonist, (iii) treating platelets from the first fraction and the second fraction of the blood sample with a PAR4 agonist in vitro, (iv) measuring platelet activation of both the first and second fractions and (v) determining the percentage inhibition of platelet activation by the PAR4 antagonist by comparing the platelet activation in the first fraction with the platelet activation in the second fraction; wherein the PAR4 agonist comprises an amino acid sequence of Formula I:
Ala-Xₐₐ₁-Pro-Gly-Xₐₐ₂-Leu-Val (Formula I)
wherein,
the amino terminus of the peptide is not fused to an amino acid;
Xₐₐ₁ is selected from Tyr and Phe(4-F);
Xaa2 is selected from Trp(5-OH), (D,L)-Trp(5-Br), D-Trp, Bzt, Tpi, His, Tza, 3-Thi, 3-Fur, His(Bzl), Phe, Tyr, Phe(penta-F), 2-Pya, 3-Pya, 4-Pya, Dpa, 3-Pya(4-Tolyl), Bip(2-Methyl), 1-Naphthyl-Ala, 2-Naphthyl-Ala, Tyr(Bzl), or Styryl-Ala; and
(−) is a peptide bond.

23. The method of claim 22, wherein the subject is treated with PAR4 antagonist if the second fraction less than 25% of the platelet activation of the first fraction.

24. The method of claim 22, wherein the subject is treated with PAR4 antagonist if the second fraction less than 10% of the platelet activation of the first fraction.

25. The method of claim 22, wherein the subject is treated with PAR4 antagonist if the second fraction less than 5% of the platelet activation of the first fraction.

26. A method for reducing or decreasing platelet function in a subject in need thereof comprising (i) submitting a platelet sample obtained from the subject for platelet activation testing with a PAR4 agonist, wherein the PAR4 agonist activates the platelets, and (ii) administering an effective amount of a PAR4 antagonist to the subject, wherein the effective amount of the PAR4 antagonist is calculated based on the report of the platelet activation and wherein the PAR4 agonist comprises an amino acid sequence of Formula I:
Ala-Xₐₐ₁-Pro-Gly-Xₐₐ₂-Leu-Val (Formula I)
wherein,
the amino terminus of the peptide is not fused to an amino acid;
Xₐₐ₁ is selected from Tyr and Phe(4-F);
Xₐₐ₂ is selected from Trp(5-OH), (D,L)-Trp(5-Br), D-Trp, Bzt, Tpi, His, Tza, 3-Thi, 3-Fur, His(Bzl), Phe, Tyr, Phe(penta-F), 2-Pya, 3-Pya, 4-Pya, Dpa, 3-Pya(4-Tolyl), Bip(2-Methyl), 1-Naphthyl-Ala, 2-Naphthyl-Ala, Tyr(Bzl), or Styryl-Ala; and
(−) is a peptide bond; and
the peptide has a PAR4 agonist activity.

27. The method of claim 26, which treats, prevents, or ameliorates a disease or condition associated with thromboembolism.

28. A method for treating, preventing, or ameliorating a disease or condition associated with thromboembolism in a subject suspected of having or being susceptible to a disease or condition associated with thromboembolism comprising (i) submitting a platelet sample obtained from the subject for platelet activation testing with a PAR4 agonist, and (ii) administering an effective amount of a PAR4 antagonist to the subject, wherein the effective amount of the PAR4 antagonist is calculated based on the report of the platelet activation and wherein the PAR4 agonist comprises an amino acid sequence of Formula I:
Ala-Xₐₐ₁-Pro-Gly-Xₐₐ₂-Leu-Val (Formula I)
wherein,
the amino terminus of the peptide is not fused to an amino acid;
Xₐₐ₁ is selected from Tyr and Phe(4-F);
Xₐₐ₂ is selected from Trp(5-OH), (D,L)-Trp(5-Br), D-Trp, Bzt, Tpi, His, Tza, 3-Thi, 3-Fur, His(Bzl), Phe, Tyr, Phe(penta-F), 2-Pya, 3-Pya, 4-Pya, Dpa, 3-Pya(4-Tolyl), Bip(2-Methyl), 1-Naphthyl-Ala, 2-Naphthyl-Ala, Tyr(Bzl), or Styryl-Ala; and
(−) is a peptide bond; and
the peptide has a PAR4 agonist activity.

29. The method of any one of claims 26 to 28, which further comprises measuring the platelet activation after the platelets have been in contact with the PAR4 agonist.

30. The method of any one of claims 26-29, wherein the disease or condition associated with thromboembolism is selected from arterial cardiovascular thromboembolic disorders, venous cardiovascular thromboembolic disorders, thromboembolic disorders in the chambers of the heart or in the peripheral circulation, arterial cerebrovascular thromboembolic disorders, venous cerebrovascular thromboembolic disorders, or any combinations thereof.

31. A method for reducing an effective amount of a PAR4 antagonist comprising (i) contacting a PAR4 agonist with platelets in a sample obtained from the subject, (ii) measuring the platelet activation by the PAR4 agonist, wherein the PAR4 agonist activates the platelets below a normal diagnostic score and (iii) providing a report to a healthcare provider, recommending that the healthcare provider reduce the effective amount of the PAR4 antagonist compared to the standard effective amount for a normal responder,
wherein the PAR4 agonist comprises an amino acid sequence of Formula I:
Ala-Xₐₐ₁-Pro-Gly-Xₐₐ₂-Leu-Val (Formula I)
wherein,
the amino terminus of the peptide is not fused to an amino acid;
Xₐₐ₁ is selected from Tyr and Phe(4-F);
Xₐₐ₂ is selected from Trp(5-OH), (D,L)-Trp(5-Br), D-Trp, Bzt, Tpi, His, Tza, 3-Thi, 3-Fur, His(Bzl), Phe, Tyr, Phe(penta-F), 2-Pya, 3-Pya, 4-Pya, Dpa, 3-Pya(4-Tolyl), Bip(2-Methyl), 1-Naphthyl-Ala, 2-Naphthyl-Ala, Tyr(Bzl), or Styryl-Ala; and
(−) is a peptide bond; and
the peptide has a PAR4 agonist activity.

32. A method for maintaining an effective amount of a PAR4 antagonist comprising (i) contacting a PAR4 agonist with platelets in a sample obtained from the subject, (ii) measuring the platelet activation by the PAR4 agonist, wherein the PAR4 agonist activates the platelets equal to or above a normal diagnostic score and below a high diagnostic score and (iii) providing a report to a healthcare provider, recommending that the healthcare provider maintain an effective amount of the PAR4 antagonist compared to the standard effective amount for a normal responder,
wherein the PAR4 agonist comprises an amino acid sequence of Formula I:
Ala-Xₐₐ₁-Pro-Gly-Xₐₐ₂-Leu-Val (Formula I)
wherein,
the amino terminus of the peptide is not fused to an amino acid;
Xₐₐ₁ is selected from Tyr and Phe(4-F);
Xₐₐ₂ is selected from Trp(5-OH), (D,L)-Trp(5-Br), D-Trp, Bzt, Tpi, His, Tza, 3-Thi, 3-Fur, His(Bzl), Phe, Tyr, Phe(penta-F), 2-Pya, 3-Pya, 4-Pya, Dpa, 3-Pya(4-Tolyl), Bip(2-Methyl), 1-Naphthyl-Ala, 2-Naphthyl-Ala, Tyr(Bzl), or Styryl-Ala; and
(−) is a peptide bond; and
the peptide has a PAR4 agonist activity.

33. A method for increasing an effective amount of a PAR4 antagonist comprising (i) contacting a PAR4 agonist with platelets in a sample obtained from the subject, (ii) measuring the platelet activation by the PAR4 agonist, wherein the PAR4 agonist activates the platelets equal to or above a high diagnostic score and (iii) providing a report to a healthcare provider, recommending that the healthcare provider increase the effective amount of the PAR4 antagonist compared to the standard effective amount for a normal responder,
wherein the PAR4 agonist comprises an amino acid sequence of Formula I:
Ala-Xₐₐ₁-Pro-Gly-Xₐₐ₂-Leu-Val (Formula I)
wherein,
the amino terminus of the peptide is not fused to an amino acid;
Xₐₐ₁ is selected from Tyr and Phe(4-F);
Xₐₐ₂ is selected from Trp(5-OH), (D,L)-Trp(5-Br), D-Trp, Bzt, Tpi, His, Tza, 3-Thi, 3-Fur, His(Bzl), Phe, Tyr, Phe(penta-F), 2-Pya, 3-Pya, 4-Pya, Dpa, 3-Pya(4-Tolyl), Bip(2-Methyl), 1-Naphthyl-Ala, 2-Naphthyl-Ala, Tyr(Bzl), or Styryl-Ala; and
(−) is a peptide bond; and
the peptide has a PAR4 agonist activity.

34. A method for administering a PAR4 antagonist to a subject, the method comprising: (i) obtaining a blood sample from a subject that has been treated with a PAR4 antagonist, (ii) treating platelets from the blood sample with a PAR4 agonist in vitro, (iii) measuring platelet activation (iv) comparing the platelet activation in the blood sample following the PAR4 antagonist treatment with the platelet activation in a blood sample obtained prior to the PAR4 antagonist treatment (v) increasing the subject's dose of the PAR4 antagonist if the subject's sample obtained after the PAR4 antagonist treatment has high activity for the PAR4 agonist, and (vi) maintaining the subject's dose of the PAR4 antagonist if the subject's sample obtained after the PAR4 antagonist treatment has low activity for the PAR4 agonist.

35. The method of claim 34, wherein the subject sample has low activity for the PAR4 agonist if the sample has less than 25% of the activity of the blood sample obtained prior to the PAR4 antagonist treatment.

36. The method of claim 34, wherein the subject sample has low activity for the PAR4 agonist if the sample has less than 15% of the activity of the blood sample obtained prior to the PAR4 antagonist treatment.

37. The method of claim 34, wherein the subject sample has low activity for the PAR4 agonist if the sample has less than 10% of the activity of the blood sample obtained prior to the PAR4 antagonist treatment.

38. The method of claim 34, wherein the subject sample has low activity for the PAR4 agonist if the sample has less than 5% of the activity of the blood sample obtained prior to the PAR4 antagonist treatment.

39. The method of claim 34, wherein the subject sample has high activity for the PAR4 agonist if the sample has at least 50% of the activity of the blood sample obtained prior to the PAR4 antagonist treatment.

40. The method of claim 34, wherein the subject sample has high activity for the PAR4 agonist if the sample has at least 75% of the activity of the blood sample obtained prior to the PAR4 antagonist treatment.

41. The method of claim 34, wherein the subject sample has high activity for the PAR4 agonist if the sample has at least 80% of the activity of the blood sample obtained prior to the PAR4 antagonist treatment.

42. The method of claim 34, wherein the subject sample has high activity for the PAR4 agonist if the sample has at least 90% of the activity of the blood sample obtained prior to the PAR4 antagonist treatment.

43. The method of any one of claims 1 to 42, wherein the platelet activation is measured by changes in the platelet cytoplasm, by changes of the platelet membrane, by changes in the levels of analytes released by platelets, by the changes in the morphology of the platelet, by the ability of platelets to form thrombi or platelet aggregates in flowing or stirred whole blood, by the ability of platelets to adhere to a static surface which is derivatised with relevant ligands (e.g., Von Willebrand Factor, Collagen, fibrinogen, or other extracellular matrix proteins or synthetic fragments of any of the proteins), or by the changes in the shape of the platelets, or any combinations thereof.

44. The method of claim 43, wherein the platelet activation is measured by changes in the levels of one or more analytes released by platelets.

45. The method of claim 44, wherein the one or more analytes related by platelets are CD62p (P-selectin), CD63, ATP, or any combination thereof.

46. The method of claim 43, wherein the platelet activation is measured by the ability of platelets to adhere to a static surface which is derivatised with one or more relevant ligands.

47. The method of claim 46, wherein the one or more relevant ligands are fibrinogen, von Willebrand factor, collagen, other extracellular matrix proteins, synthetic fragment thereof, or any combination thereof.

48. The method of claim 43, wherein the platelet activation is measured by the degree of phosphorylation of vasodilator-stimulated phosphoprotein (VASP) upon platelet activation.

49. The method of claim 43, wherein the platelet activation is measured by the level of platelet-leukocyte aggregates.

50. The method of any one of claims 6, 8 to 10, 16, 17, 20, 21, 28 and 29, wherein the normal diagnostic score is the lower 10%, 12.5%, 15%, 20%, 25%, 30%, 35%, 40%, 45%, or 49% of the distribution of platelet activation measurement results observed in a population sample.

51. The method of any one of claims 6, 8 to 10, 16, 17, 20, 21, 28 and 29, wherein the normal diagnostic score is the lower 25% of the distribution of platelet activation measurement results observed in a population sample.

52. The method of any one of claims 7 to 10, 15, 17, 19, 21, 28 and 29, wherein the high diagnostic score is the higher 51%, 55%, 60%, 65%, 70%, 75%, 80%, 85%, 87.5%, or 90% of the distribution of platelet activation measurement results observed in a population sample.

53. The method of any one of claims 7 to 10, 15, 17, 19, 21, 28 and 29, wherein the high diagnostic score is the higher 75% of the distribution of platelet activation measurement results observed in a population sample.

54. The method of any one of claims 50 to 53, wherein the platelet activation measurement results observed in the population sample is FIG. 2B.

55. The method of claim 45 or 46, wherein the platelet activation is detected by an antibody specifically binding to the analytes or ligands.

56. The method of claim 55, wherein the antibody is conjugated to fluorescein isothiocyanate (FITC) or phycoerythrin (PE).

57. The method of claim 55 or 56, wherein the antibody is a polyclonal antibody or a monoclonal antibody.

58. The method of any one of claims 55 to 57, wherein the antibody is an anti-fibrinogen antibody.

59. The method of claim 58, wherein the anti-fibrinogen antibody is selected from F0111, LS-B2573, LS-B697, LS-B5249, LS-B381, LS-B3048, LS-B7075, LS-052057, LS-C109177, LS-C150799, MCA2760, 4440-8004, NBP1-33582, NB600-926, NBP1-47442, NBP2-11515, NB120-10070, NBP1-96183, NBP1-96180, or any combinations thereof.

60. The method of claim 58 or 59, wherein the anti-fibrinogen antibody is a rabbit anti-human fibrinogen polyclonal antibody.

61. The method of any one of claims 58 to 60, wherein the normal diagnostic score comprises a percentage of platelet activation measured by F0111, which is about 20%, about 25%, about 30%, about 35%, about 40%, about 45%, or about 50% platelets being activated.

62. The method of any one of claims 58 to 60, wherein the high diagnostic score comprises a percentage of platelet activation measured by F0111, which is about 40%, about 45%, about 50%, about 55%, about 60%, about 65%, about 70%, about 75%, about 80%, about 85%, about 90%, about 95% platelets being activated.

63. The method of any one of claims 58 to 60, wherein the normal diagnostic score comprises a percentage of platelet activation, which is about 20%, and the high diagnostic score comprises a percentage of platelet activation, which is about 50%.

64. The method of any one of claims 58 to 60, wherein the normal diagnostic score comprises a percentage of platelet activation, which is about 30%, and the high diagnostic score comprises a percentage of platelet activation, which is about 50%.

65. The method of any one of claims 58 to 60, wherein the normal diagnostic score comprises a percentage of platelet activation, which is about 40%, and the high diagnostic score comprises a percentage of platelet activation, which is about 60%.

66. The method of any one of claims 55 to 57, wherein the antibody is an anti-P-selectin antibody specifically binding to P-selectin.

67. The method of claim 66, wherein the antibody is an anti-P-selectin antibody selected from CLB-Thromb/6, AK4, 1E3, CTB201, P8G6, MAB2154, ab91132, LS-B3578, LS-B3656, LS-C134593, LS-C13963, 10-667-C100, 11-450-C100, 1A-450-T100, 1Y-450-T100, OASA02343, PN IM1315, or any combinations thereof.

68. The method of claim 67, wherein the anti-P-selectin antibody is CLB-Thromb/6.

69. The method of any one of claims 66 to 68, wherein the normal diagnostic score comprises a percentage of platelet activation measured by CLB-Thromb/6, which is about 60%, about 65%, about 70%, about 75%, or about 80%.

70. The method of claim 69, wherein the high diagnostic score comprises a percentage of platelet activation measured by CLB-Thromb/6, which is about 85%, 90%, 95%, or 99%.

71. A kit for identifying a subject who is responsive to PAR4 antagonist therapy comprising a PAR4 agonist and an instructional material, which instructs a healthcare provider to mix the PAR4 agonist with platelets in a sample taken from a subject in need of PAR4 antagonist therapy and to measure the platelet activation, wherein the PAR4 agonist comprises an amino acid sequence of Formula I:
Ala-Xₐₐ₁-Pro-Gly-Xₐₐ₂-Leu-Val (Formula I)
wherein,
the amino terminus of the peptide is not fused to an amino acid;
Xₐₐ₁ is selected from Tyr and Phe(4-F);
Xₐₐ₂ is selected from Trp(5-OH), (D,L)-Trp(5-Br), D-Trp, Bzt, Tpi, His, Tza, 3-Thi, 3-Fur, His(Bzl), Phe, Tyr, Phe(penta-F), 2-Pya, 3-Pya, 4-Pya, Dpa, 3-Pya(4-Tolyl), Bip(2-Methyl), 1-Naphthyl-Ala, 2-Naphthyl-Ala, Tyr(Bzl), or Styryl-Ala; and
(−) is a peptide bond; and
the peptide has a PAR4 agonist activity.

72. The kit of claim 71, which further comprises an agent to measure platelet activation.

73. The kit of claim 72, wherein the agent to measure platelet activation can detect a platelet marker, which is expressed at different levels on resting and activated platelets.

74. The kit of claim 73, wherein the marker is selected from: GlycoProtein (GP)IIb/IIIa, fibrinogen binding to GPIIb/IIIa or other membrane structures, GPIa/IIa (VLA-2), binding of collagen or collagen mimetics or to other membrane structures, CD62P (P-selectin), CD63, GPIb-alpha, GPIb-beta, GP IX, GPVI, GPIV, CD43, CD100, CD147, annexin V, lactadherin, or any combinations thereof.

75. The kit of claim 72, wherein the agent to measure platelet activation can detect changes in the levels of analytes released by platelets.

76. The kit of claim 72 or 74, wherein the agent comprises an antibody specifically binding to the analytes.

77. The kit of claim 76, wherein the analytes are P-selectin.

78. The kit of claim 72, wherein the agent to measure platelet activation is an anti-P-selectin antibody.

79. The kit of claim 72, wherein the agent to measure platelet activation is an anti-fibrinogen antibody.

80. The kit of claim 78 or 79, wherein the antibody is conjugated to fluorescein isothiocyanate (FITC) or phycoerythrin (PE).

81. The kit of claim 80, wherein the antibody is a polyclonal antibody or a monoclonal antibody.

82. The kit of claim 79, wherein the anti-fibrinogen antibody is selected from F-0111, LS-B2573, LS-B697, LS-B5249, LS-B381, LS-B3048, LS-B7075, LS-052057, LS-C109177, LS-C150799, MCA2760, 4440-8004, NBP1-33582, NB600-926, NBP1-47442, NBP2-11515, NB120-10070, NBP1-96183, NBP1-96180, or any combinations thereof.

83. The kit of claim 79 or 82, wherein the anti-fibrinogen antibody is a rabbit anti-human fibrinogen polyclonal antibody.

84. The kit of claim 78 wherein the antibody is an anti-P-selectin antibody specifically binding to P-selectin.

85. The kit of claim 84, wherein the anti-P-selectin antibody selected from CLB-Thromb/6, AK4, 1E3, CTB201, P8G6, MAB2154, ab91132, LS-B3578, LS-B3656, LS-C134593, LS-C13963, 10-667-C100, 11-450-C100, 1A-450-T100, 1Y-450-T100, OASA02343, PN IM1315, or any combinations thereof.

86. The kit of claim 84, wherein the anti-P-selectin antibody is CLB-Thromb/6.

87. The method of any one of claim 1 to or the kit of any one of claims 60 to 75, wherein Xₐₐ₁ of the PAR4 agonist is Phe(4-F).

88. The method of any one of claims 1 to 59 or the kit of any one of claims 60 to 76, wherein Xₐₐ₂ of the PAR4 agonist is Trp.

89. The method of any one of claims 1 to 59 or the kit of any one of claims 60 to 77, wherein the PAR4 agonist comprises the amino acid sequence of SEQ ID NO: 3.

90. The method of any one of claims 1 to 59 or the kit of any one of claims 60 to 78, wherein the PAR4 agonist further comprises Lys after Val.

91. The method of any one of claims 1 to 59 or the kit of any one of claims 60 to 79, wherein the PAR4 agonist further comprises Lys-Asn after Val.

92. The method of any one of claims 1 to 59 or the kit of any one of claims 60 to 80, wherein the PAR4 agonist further comprises Lys-Asn-Gly after Val.

93. The method of any one of claims 1 to 59 or the kit of any one of claims 60 to 81, wherein the PAR4 agonist consists essentially of or consists of the amino acid sequence.

94. The method of any one of claims 1 to 59 or the kit of any one of claims 60 to 82, wherein the C-terminus of the PAR4 agonist is amidated.


SEQUENCE LISTING (39 sequences)

SEQ ID NO: 1 - protein, 6 aa, Artificial
  synthetic peptide
AYPGKF

SEQ ID NO: 2 - protein, 10 aa, Artificial
  synthetic peptide
AYPGWLVKNG

SEQ ID NO: 3 - protein, 10 aa, Artificial
  synthetic peptide
AXPGWLVKNG

SEQ ID NO: 4 - protein, 9 aa, Artificial
  synthetic peptide
AXPGWLVKN

SEQ ID NO: 5 - protein, 8 aa, Artificial
  synthetic peptide
AXPGWLVK

SEQ ID NO: 6 - protein, 7 aa, Artificial
  synthetic peptide
AXPGWLV

SEQ ID NO: 7 - protein, 6 aa, Artificial
  synthetic peptide
AXPGWL

SEQ ID NO: 8 - protein, 5 aa, Artificial
  synthetic peptide
AXPGW

SEQ ID NO: 9 - protein, 4 aa, Artificial
  synthetic peptide
AXPG

SEQ ID NO: 10 - protein, 4 aa, Artificial
  synthetic peptide
AYPG

SEQ ID NO: 11 - protein, 10 aa, Artificial
  synthetic peptide
AYPGQVCAND

SEQ ID NO: 12 - protein, 7 aa, Artificial
  synthetic peptide
AXPGXLV

SEQ ID NO: 13 - protein, 7 aa, Artificial
  synthetic peptide
AXPGXLV

SEQ ID NO: 14 - protein, 7 aa, Artificial
  synthetic peptide
AXPGXLV

SEQ ID NO: 15 - protein, 7 aa, Artificial
  synthetic peptide
AXPGXLV

SEQ ID NO: 16 - protein, 7 aa, Artificial
  synthetic peptide
AXPGXLV

SEQ ID NO: 17 - protein, 7 aa, Artificial
  synthetic peptide
AXPGHLV

SEQ ID NO: 18 - protein, 7 aa, Artificial
  synthetic peptide
AXPGXLV

SEQ ID NO: 19 - protein, 7 aa, Artificial
  synthetic peptide
AXPGXLV

SEQ ID NO: 20 - protein, 7 aa, Artificial
  synthetic peptide
AXPGXLV

SEQ ID NO: 21 - protein, 7 aa, Artificial
  synthetic peptide
AXPGXLV

SEQ ID NO: 22 - protein, 7 aa, Artificial
  synthetic peptide
AXPGFLV

SEQ ID NO: 23 - protein, 7 aa, Artificial
  synthetic peptide
AXPGYLV

SEQ ID NO: 24 - protein, 7 aa, Artificial
  synthetic peptide
AXPGXLV

SEQ ID NO: 25 - protein, 7 aa, Artificial
  synthetic peptide
AXPGXLV

SEQ ID NO: 26 - protein, 7 aa, Artificial
  synthetic peptide
AXPGXLV

SEQ ID NO: 27 - protein, 7 aa, Artificial
  synthetic peptide
AXPGXLV

SEQ ID NO: 28 - protein, 7 aa, Artificial
  synthetic peptide
AXPGXLV

SEQ ID NO: 29 - protein, 7 aa, Artificial
  synthetic peptide
AXPGXLV

SEQ ID NO: 30 - protein, 7 aa, Artificial
  synthetic peptide
AXPGXLV

SEQ ID NO: 31 - protein, 7 aa, Artificial
  synthetic peptide
AXPGXLV

SEQ ID NO: 32 - protein, 7 aa, Artificial
  synthetic peptide
AXPGXLV

SEQ ID NO: 33 - protein, 7 aa, Artificial
  synthetic peptide
AXPGXLV

SEQ ID NO: 34 - protein, 7 aa, Artificial
  synthetic peptide
AXPGXLV

SEQ ID NO: 35 - protein, 866 aa, Homo sapiens
  SIGNAL (1)..(19)
  DOMAIN (623)..(864): C-terminal domain
MFSMRIVCLVLSVVGTAWTADSGEGDFLAEGGGVRGPRVVERHQSACKDSDWPFCSDEDWNYKCPSGCRMKGLIDEVNQDFTNRINKLKNSLFEYQKNNKDSHSLTTNIMEILRGDFSSANNRDNTYNRVSEDLRSRIEVLKRKVIEKVQHIQLLQKNVRAQLVDMKRLEVDIDIKIRSCRGSCSRALAREVDLKDYEDQQKQLEQVIAKDLLPSRDRQHLPLIKMKPVPDLVPGNFKSQLQKVPPEWKALTDMPQMRMELERPGGNEITRGGSTSYGTGSETESPRNPSSAGSWNSGSSGPGSTGNRNPGSSGTGGTATWKPGSSGPGSTGSWNSGSSGTGSTGNQNPGSPRPGSTGTWNPGSSERGSAGHWTSESSVSGSTGQWHSESGSFRPDSPGSGNARPNNPDWGTFEEVSGNVSPGTRREYHTEKLVTSKGDKELRTGKEKVTSGSTTTTRRSCSKTVTKTVIGPDGHKEVTKEVVTSEDGSDCPEAMDLGTLSGIGTLDGFRHRHPDEAAFFDTASTGKTFPGFFSPMLGEFVSETESRGSESGIFTNTKESSSHHPGIAEFPSRGKSSSYSKQFTSSTSYNRGDSTFESKSYKMADEAGSEADHEGTHSTKRGHAKSRPVRDCDDVLQTHPSGTQSGIFNIKLPGSSKIFSVYCDQETSLGGWLLIQQRMDGSLNFNRTWQDYKRGFGSLNDEGEGEFWLGNDYLHLLTQRGSVLRVELEDWAGNEAYAEYHFRVGSEAEGYALQVSSYEGTAGDALIEGSVEEGAEYTSHNNMQFSTFDRDADQWEENCAEVYGGGWWYNNCQAANLNGIYYPGGSYDPRNNSPYEIENGVVWVSFRGADYSLRAVRMKIRPLVTQ

SEQ ID NO: 36 - protein, 491 aa, Homo sapiens
  SIGNAL (1)..(30)
  DOMAIN (232)..(488): C-terminal domain
MKRMVSWSFHKLKTMKHLLLLLLCVFLVKSQGVNDNEEGFFSARGHRPLDKKREEAPSLRPAPPPISGGGYRARPAKAAATQKKVERKAPDAGGCLHADPDLGVLCPTGCQLQEALLQQERPIRNSVDELNNNVEAVSQTSSSSFQYMYLLKDLWQKRQKQVKDNENVVNEYSSELEKHQLYIDETVNSNIPTNLRVLRSILENLRSKIQKLESDVSAQMEYCRTPCTVSCNIPVVSGKECEEIIRKGGETSEMYLIQPDSSVKPYRVYCDMNTENGGWTVIQNRQDGSVDFGRKWDPYKQGFGNVATNTDGKNYCGLPGEYWLGNDKISQLTRMGPTELLIEMEDWKGDKVKAHYGGFTVQNEANKYQISVNKYRGTAGNALMDGASQLMGENRTMTIHNGMFFSTYDRDNDGWLTSDPRKQCSKEDGGGWWYNRCHAANPNGRYYWGGQYTWDMAKHGTDDGVVWMNWKGSWYSMRKMSMKIRPFFPQQ

SEQ ID NO: 37 - protein, 453 aa, Homo sapiens
  SIGNAL (1)..(26)
  DOMAIN (170)..(416): C-terminal domain
MSWSLHPRNLILYFYALLFLSSTCVAYVATRDNCCILDERFGSYCPTTCGIADFLSTYQTKVDKDLQSLEDILHQVENKTSEVKQLIKAIQLTYNPDESSKPNMIDAATLKSRKMLEEIMKYEASILTHDSSIRYLQEIYNSNNQKIVNLKEKVAQLEAQCQEPCKDTVQIHDITGKDCQDIANKGAKQSGLYFIKPLKANQQFLVYCEIDGSGNGWTVFQKRLDGSVDFKKNWIQYKEGFGHLSPTGTTEFWLGNEKIHLISTQSAIPYALRVELEDWNGRTSTADYAMFKVGPEADKYRLTYAYFAGGDAGDAFDGFDFGDDPSDKFFTSHNGMQFSTWDNDNDKFEGNCAEQDGSGWWMNKCHAGHLNGVYYQGGTYSKASTPNGYDNGIIWATWKTRWYSMKKTTMKIIPFNRLTIGEGQQHHLGGAKQVRPEHPAETEYDSLYPEDDL

SEQ ID NO: 38 - protein, 830 aa, Homo sapiens
MANCQIAILYQRFQRVVFGISQLLCFSALISELTNQKEVAAWTYHYSTKAYSWNISRKYCQNRYTDLVAIQNKNEIDYLNKVLPYYSSYYWIGIRKNNKTWTWVGTKKALTNEAENWADNEPNNKRNNEDCVEIYIKSPSAPGKWNDEHCLKKKHALCYTASCQDMSCSKQGECLETIGNYTCSCYPGFYGPECEYVRECGELELPQHVLMNCSHPLGNFSFNSQCSFHCTDGYQVNGPSKLECLASGIWTNKPPQCLAAQCPPLKIPERGNMTCLHSAKAFQHQSSCSFSCEEGFALVGPEVVQCTASGVWTAPAPVCKAVQCQHLEAPSEGTMDCVHPLTAFAYGSSCKFECQPGYRVRGLDMLRCIDSGHWSAPLPTCEAISCEPLESPVHGSMDCSPSLRAFQYDTNCSFRCAEGFMLRGADIVRCDNLGQWTAPAPVCQALQCQDLPVPNEARVNCSHPFGAFRYQSVCSFTCNEGLLLVGASVLQCLATGNWNSVPPECQAIPCTPLLSPQNGTMTCVQPLGSSSYKSTCQFICDEGYSLSGPERLDCTRSGRWTDSPPMCEAIKCPELFAPEQGSLDCSDTRGEFNVGSTCHFSCDNGFKLEGPNNVECTTSGRWSATPPTCKGIASLPTPGLQCPALTTPGQGTMYCRHHPGTFGFNTTCYFGCNAGFTLIGDSTLSCRPSGQWTAVTPACRAVKCSELHVNKPIAMNCSNLWGNFSYGSICSFHCLEGQLLNGSAQTACQENGHWSTTVPTCQAGPLTIQEALTYFGGAVASTIGLIMGGTLLALLRKRFRQKDDGKCPLNPHSHLGTYGVFTNAAFDPSP

SEQ ID NO: 39 - protein, 6 aa, Artificial
  synthetic peptide
SFFLRR